(19) United States
(12) Patent Application Publication
(10) Pub. No.: US 2023/0052022 A1
(43) Pub. Date: Feb. 16, 2023

(54) Systems and Methods for Dynamic Charting

(71) Applicant: HEALTHPOINTE SOLUTIONS, INC., Austin, TX (US)

(72) Inventors: Nathan GNANASAMBANDAM, Irvine, CA (US); Mark Henry ANDERSON, Newport Coast, CA (US)

(73) Assignee: HEALTHPOINTE SOLUTIONS, INC., Austin, TX (US)

(21) Appl. No.: 17/794,174

(22) PCT Filed: Jan. 21, 2021
(86) PCT No.: PCT/US2021/014353

§ 371 Date: Jul. 20, 2022

Related U.S. Application Data
(60) Provisional application No. 62/964,502, filed on Jan. 22, 2020.

(51) Int. Cl. G06N 3/00; G06N 20/00

(52) U.S. Cl. CPC G06N 3/004; G06N 20/00

(57) ABSTRACT

A device receives patient data that indicates health related information associated with a patient. The device identifies, by processing the patient data using one or more natural language processing techniques, indicia associated with a health status of the patient. The device identifies similarities between the indicia and the content. The device generates, using an artificial intelligence engine, cognified data based on the similarities. The device identifies a medical code that correlates to particular content that is similar to the indicia. The device causes the cognified data to be displayed in association with medical code.

### CROSS-REFERENCE TO RELATED APPLICATIONS

This application claims the benefit of U.S. Provisional Application Ser. No. 62/964,502 filed Jan. 22, 2020 titled “Systems and Methods for Dynamic Charting,” which provisional application is incorporated by reference herein as if reproduced in full below.

### BACKGROUND

Population health management entails aggregating patient data across multiple health information technology resources, analyzing the data with reference to a single patient, and generating actionable items through which care providers can improve both clinical and financial outcomes. A population health management service seeks to improve the health outcomes of a group by improving clinical outcomes while lowering costs.

### SUMMARY

This section provides a general summary of the present disclosure and is not a comprehensive disclosure of its full scope or all of its features, aspects, and objectives.

Disclosed herein are implementations of a method for receiving patient data that indicates health related information associated with a patient, identifying, by processing the patient data using one or more natural language processing techniques, indicia associated with a health status of the patient, identifying similarities between the indicia and content that is part of a corpus of health related data, generating, using an artificial intelligence engine, cognified data based on the similarities, identifying a medical code that correlates to particular content that is similar to the indicia, and causing the cognified data to be displayed in association with the medical code.

Also disclosed herein are implementations of a device that includes one or more processors and one or more memories including instructions that, when executed by the one or more processors, cause the one or more processors to receive patient data that indicates health related information associated with a patient, to identify, by processing the patient data using one or more natural language processing techniques, indicia associated with a health status of the patient, to identify similarities between the indicia and content that is part of a corpus of health related data, to generate, using an artificial intelligence engine, cognified data based on the similarities, to identify a medical code that correlates to particular content that is similar to the indicia, and to cause the cognified data to be displayed in association with the medical code.

Also disclosed herein is a non-transitory computer-readable medium storing instructions that, when executed by one or more processors, cause the one or more processors to receive patient data that indicates health related information associated with a patient, to identify, by processing the patient data using one or more natural language processing techniques, indicia associated with a health status of the patient, to identify similarities between the indicia and content that is part of a corpus of health related data, to generate, using an artificial intelligence engine, cognified data based on the similarities, to identify a medical code that correlates to particular content that is similar to the indicia, and to cause the cognified data to be displayed in association with the medical code.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 illustrates, in block diagram form, a system architecture 100 that can be configured to provide a population health management service, in accordance with various embodiments.

FIG. 2 shows additional details of a knowledge cloud, in accordance with various embodiments.

FIG. 3 shows an example subject matter ontology, in accordance with various embodiments.

FIG. 4 shows aspects of a conversation, in accordance with various embodiments.

FIG. 5 shows a cognitive map or “knowledge graph”, in accordance with various embodiments.

FIG. 6 illustrates a detailed view of a computing device that can represent the computing devices of FIG. 1 used to implement the various platforms and techniques described herein, according to some embodiments.

FIG. 7 shows a method for cognifying unstructured data, in accordance with various embodiments.

FIG. 8 shows a method for identifying missing information in a corpus of health related data, in accordance with various embodiments.

FIG. 9 shows a method for using feedback pertaining to the accuracy of cognified data to update an artificial intelligence engine, in accordance with various embodiments.

FIG. 10A shows a block diagram for using a knowledge graph to generate possible health related information, in accordance with various embodiments.

FIG. 10B shows a block diagram for using a logical structure to identify structural similarities with known predicates to generate cognified data, in accordance with various embodiments.

FIG. 11 shows a method for providing first information pertaining to a possible medical condition of a patient to a computing device, in accordance with various embodiments.

FIG. 12 shows a method for providing second and third information pertaining to a possible medical condition of a patient to a computing device, in accordance with various embodiments.

FIG. 13 shows a method for providing second information pertaining to a second possible medical condition of the patient, in accordance with various embodiments.

FIG. 14 shows an example of providing first information of a knowledge graph representing a possible medical condition, in accordance with various embodiments.

FIG. 15 shows an example of providing second information of the knowledge graph representing the possible medical condition, in accordance with various embodiments.

FIG. 16 shows an example of providing third information of the knowledge graph representing the possible medical condition, in accordance with various embodiments.

FIG. 17 shows a method for using cognified data to diagnose a patient, in accordance with various embodiments.

FIG. 18 shows a method for determining a severity of a medical condition based on a stage and a type of the medical condition, in accordance with various embodiments.

FIG. 19 shows an example of a knowledge graph, a patient graph, and a care plan, in accordance with various embodiments.

FIGS. 207A-20C show examples for generating a care plan using a knowledge graph and a patient graph, in accordance with various embodiments.

FIGS. 21A-21H are diagrams of one or more example embodiments described herein.

FIG. 22 shows a method for generating cognified data and causing the cognified data to be displayed in association with related medical codes, in accordance with various embodiments.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

According to some embodiments, a cognitive intelligence platform integrates and consolidates data from various sources and entities and provides a population health management service. The cognitive intelligence platform has the ability to extract concepts, relationships, and draw conclusions from a given text posed in natural language (e.g., a passage, a sentence, a phrase, and a question) by performing conversational analysis which includes analyzing conversational context. For example, the cognitive intelligence platform has the ability to identify the relevance of a posed question to another question.

The benefits provided by the cognitive intelligence platform, in the context of healthcare, include freeing up physicians from focusing on day to day population health management. Thus a physician can focus on her core competency—which includes disease/risk diagnosis and prognosis and patient care. The cognitive intelligence platform provides the functionality of a health coach and includes a physician's directions in accordance with the medical community's recommended care protocols and also builds a systemic knowledge base for health management.

Accordingly, the cognitive intelligence platform implements an intuitive conversational cognitive agent that engages in a question and answering system that is human-like in tone and response. The described cognitive intelligence platform endeavors to compassionately solve goals, questions and challenges.

In addition, physicians often generate patient notes before, during, and/or after consultation with a patient. The patient notes may be included in an electronic medical record (EMR). When a patient returns for a subsequent visit, the physician may review numerous EMRs for the patient. Such a review process may be time consuming and inefficient. Insights may be hidden in the various EMRs and may result in the physician making an incorrect diagnosis. Further, it may involve the physician accessing numerous screens and performing multiple queries on a database to obtain the various EMRs. As a result, the computing device of the physician may waste computing resources by loading various screens and sending requests for EMR data to a server. The server that receives the requests may also waste computing resources by processing the numerous requests and transmitting numerous responses. In addition, network resources may be wasted by transmitting the requests and responses between the server and the client.

Accordingly, some embodiments of the present disclosure address the issues of reviewing the EMRs, by cognifying unstructured data. Unstructured data may include patient notes entered into one or more EMRs by a physician. The patient notes may explain symptoms described by the patient or detected by the physician, vital signs, recommended treatment, risks, prior health conditions, familial health history, and the like. The patient notes may include numerous strings of characters arranged into sentences. The sentences may be organized in one or more paragraphs. The sentences may be parsed and indicia may be identified. The indicia may include predicates, objectives, nouns, verbs, cardinals, ranges, keywords, phrases, numbers, concepts, or some combination thereof.

The indicia may be compared to one or more knowledge graphs that each represents health related information (e.g., a disease) and various characteristics of the health related information. The knowledge graph may also include how the various diseases are related to one another (e.g., bronchitis can lead to pneumonia). The knowledge graph may represent a model that includes individual elements (nodes) and predicates that describe properties and/or relationships between those individual elements. A logical structure (e.g., Nth order logic) may underlie the knowledge graph that uses the predicates to connect various individual elements. The knowledge graph and the logical structure may combine to form a language that recites facts, concepts, correlations, conclusions, propositions, and the like. The knowledge graph and the logical structure may be generated and updated continuously or on a periodic basis by an artificial intelligence engine with evidence-based guidelines, physician research, patient notes in EMRs, physician feedback, and so forth. The predicates and individual elements may be generated based on data that is input to the artificial intelligence engine. The data may include evidence-based guidelines that is obtained from a trusted source, such as a physician. The artificial intelligence engine may continuously learn based on input data (e.g., evidence-based guidelines, clinical trials, physician research, electronic medical records, etc.) and modify the individual elements and predicates.

For example, a physician may indicate that if a person has a blood sugar level of a certain amount and various other symptoms (e.g., unexplained weight loss, sweating, etc.), then that person has type 2 diabetes mellitus. Such a conclusion may be modeled in the knowledge graph and the logical structure as “Type 2 diabetes mellitus has symptoms of a blood sugar level of the certain amount and various other symptoms,” where “Type 2 diabetes mellitus,” “a blood sugar level of the certain amount,” and “various other symptoms” are individual elements in the knowledge graph, and “has symptoms of” is a predicate of the logical structure that relates the individual element “Type 2 diabetes mellitus” to the individual elements of “a blood sugar level of the certain amount” and “various other symptoms”.

The indicia extracted from the unstructured data may be correlated with one or more closely matching knowledge graphs by comparing similarities between the indicia and the individual elements. Tags related to possible health related information may be generated and associated with the indicia in the unstructured data. For example, the tags may specify “A leads to B” (where A is a health related information and B is another health related information), “B causes C” (where C is yet another health related information), “C has complications of D” (where D is yet another health related information), and so forth. These tags associated with the indicia may be correlated with the logical structure (e.g., predicates of the logical structure) based on structural similarity to generate cognified data. For example, if a person exhibits certain symptoms and has certain laboratory tests performed, then that person may have a certain medical condition (e.g., type 2 diabetes mellitus) that is identified in the knowledge graphs using the logical structures.

A pattern may be detected by identifying structural similarities between the tags and the logical structure in order to generate the cognified data. Cognification may refer to instilling intelligence into something. In the present disclosure, unstructured data may be cognified into cognified data by instilling intelligence into the unstructured data using the knowledge graph and the logical structure. The cognified data may include a summary of a health related condition of a patient, where the summary includes insights, conclusions, recommendations, identified gaps (e.g., in treatment, risk, quality of care, guidelines, etc.), and so forth.

The cognified data may be presented on a computing device of a physician. Instead of reading pages and pages of digital medical charts (EMRs) for a patient, the physician may read the cognified data that presents pointed summarized information that can be utilized to more efficiently and effectively treat the patient. As a result, computing resources may be saved by preventing numerous searches for EMRs and preventing accessing numerous screens displaying the EMRs. In some embodiments, the physician may submit feedback pertaining to whether or not the cognified data is accurate for the patient. The feedback may be used to update the artificial intelligence engine that uses the knowledge graph and logical structure to generate the cognified data.

In some embodiments, the cognified data may be used to diagnose a medical condition of the patient. For example, the medical condition may be diagnosed if a threshold criteria is satisfied. The threshold criteria may include matching a certain number of predicates and tags for a particular medical condition represented by a particular knowledge graph. The computing device of the physician and/or the patient may present the diagnosis and a degree of certainty based on the threshold criteria. In some embodiments, the physician may submit feedback pertaining to whether or not the diagnosis is accurate for the patient. The feedback may be used to update the artificial intelligence engine that uses the knowledge graph and logical structure to generate the diagnosis using the cognified data.

Further, patients may be inundated with information about a particular medical condition with which they are diagnosed and/or inquiring about. The information may not be relevant to a particular stage of the medical condition. The amount of information may waste memory resources of the computing device of the patient. Also, the user may have a bad experience using the computing device due to the overwhelming amount of information.

In some embodiments, user experience of using a computing device may be enhanced by running an application that performs various techniques described herein. The user may be interacting with the cognitive agent and the cognitive agent may be steering the conversation as described herein. In some embodiments, the cognitive agent may provide recommendations based on the text entered by the user, and/or patient notes in EMRs, which may be transformed into cognified data. The application may present health related information, such as the cognified data, pertaining to the medical condition to the computing device of the patient and/or the physician.

Instead of overwhelming the patient with massive amounts of information about the medical condition, the distribution of information may be regulated to the computing device of the patient and/or the physician. For example, if the patient is diagnosed as having type 2 diabetes mellitus, a controlled traversing of the knowledge graph associated with type 2 diabetes mellitus may be performed to provide information to the patient. The traversal may begin at a root node of the knowledge graph and first health related information may be provided to the computing device of the patient at a first time. The first health related information may pertain to a name of the medical condition, a definition of the possible medical condition, or some combination thereof. At a second time, health related information associated with a second node of the knowledge graph may be provided to the computing device of the patient. The second health related information may pertain to how the medical condition affects people, signs and symptoms of the medical condition, a way to treat the medical condition, complications of the medical condition, a progression of the medical condition, or some combination thereof. The health related information associated with the remaining nodes in the knowledge graph may be distributed to the computing device of the patient at different respective times. In some embodiments, the health related information to be provided and/or the times at which the health related information is provided may be selected based on relevancy to a stage of the medical condition of the patient.

In other scenarios, users (also referred to as patients herein) may use various computing devices (e.g., smartphone, tablet, laptop, etc.) to schedule an appointment with a person (also referred to as care providers herein) having a particular specialty to perform a service. For example, a patient may schedule appointments with care providers to provide one or more services to the patient. A patient may call an office where the care provider having a specialty works and speak to a person who finds an available appointment to book for the care provider and the patient. To book an appointment with another care provider having a different specialty, the patient may call the office of the other care provider having the different specialty to book an available appointment. Further, to book an appointment with a care provider for a dependent (e.g., child), the parent/guardian may contact yet another office where a care provider having yet another specialty (e.g., pediatrician) works to book an appointment. In some instances, the patient may access multiple different websites associated with the care providers to attempt to schedule an appointment. This is inconvenient for the patient and wastes resources by making multiple phone calls or accessing multiple different websites. Switching between websites to find contact information for people having different specialties may cause undesirable network, computing, and/or memory usage to occur. Additionally, typical software applications do not include functionality for scheduling appointments for an entire family (e.g., primary, spouse, dependents (children, senior citizens)) covered by an insurance plan, and/or functionality for scheduling multiple appointments for the same patient and/or different patients.

When the patient arrives for the scheduled appointments, the patient typically has to fill out paper check-in documents at each office. Even when the information requested by the check-in documents is redundant, such as medical history information, medication information, etc., various offices still request the same information. Part of the issue is a lack of interoperability of electronic medical records systems. Also, when a computing device is used to complete the check-in documents, the check-in documents are not shared with other systems associated with other specialties, and the user may have to reenter their information using a computing device of another system associated with the other specialties. As such, computing resources of the computing devices may be wasted by running an application to enable entry of information into the check-in documents, instead of just sharing the already completed check-in documents with requesting systems.

Once check-in is complete, the patient may be presented with paper reading materials in a waiting room. The reading materials may include information (e.g., symptoms, causes, treatments, etc.) pertaining to various different medical conditions. It can oftentimes be overwhelming to a patient to be presented with too much information, especially when the information does not pertain to the condition or conditions for which the patient is seeking treatment. Further, even if the patient knows what he or she is looking for, searching for the paper reading material is inefficient. To that end, even if the user finds reading material that discusses a desired topic, there typically is not a guarantee the reading material was authored/reviewed by a person having proper credentials (e.g., a medical doctor). Educating the patient with pertinent curated content that is tailored for the patient is desired.

Accordingly, some embodiments of the present disclosure address the above-identified issues, among other things. For example, an autonomous multipurpose application may execute in a cognitive intelligence platform. In some embodiments, the autonomous multipurpose application may be implemented as one or more application programming interfaces (API) executing via one or more computing devices (e.g., servers), as described in more detail below. The term “autonomous” used in conjunction with the “multipurpose application” may refer to the multipurpose application executing a set of operations on behalf of a person or another application with some degree of independence or autonomy in an intelligent manner using knowledge or representation of a user's goals or desires. The terms “autonomous multipurpose application” and “cognitive agent” may be used interchangeably herein.

In some embodiments, the autonomous multipurpose application may present different user interfaces based on a role associated with a person that logs into the autonomous multipurpose application. The various roles may include a medical personnel (e.g., medical doctor, physician, nurse, dentist, optometrist, psychiatrist, behavioral specialist, physician assistant, and the like), an administrator, a patient/user, and so forth. The user interface presented on a computing device when a person having the medical personnel role is logged in may be referred to as “clinic viewer” herein. The user interface presented on a computing device when a person having the administrator role is logged in may be referred to as “administrator viewer” herein. The user interface presented on a computing device when a person having the patient/user role may be referred to as “patient viewer” herein.

The autonomous multipurpose application may perform numerous operations pertaining to scheduling appointments for patients, checking-in patients for scheduled appointments, educating the patients about medical conditions, and/or searching for content based on search queries, among other things. For scheduling purposes, the autonomous multipurpose application may be communicatively coupled with computing devices of care providers (e.g., medical personnel) and/or electronic medical record (EMR) systems used by the care providers (e.g., medical personnel). These computing devices and/or electronic medical record systems may execute patient management systems or scheduling management systems that maintain schedules of appointments for the care providers. For example, a schedule for a care provider may show which appointments are scheduled or booked and which appointments are available by date and time.

The autonomous multipurpose application may obtain the schedules for people having a desired specialty within a certain geographic location (e.g., within a radius of a geolocation of a computing device of the user, within a radius of an entered address, etc.). A user may elect to enable electronic scheduling. If an available appointment is found within the certain geographic region, and the user is available at the same date and time as the available appointment, the autonomous multipurpose application may electronically schedule the available appointment as a booked appointment. If the user has not enabled electronic scheduling, the autonomous multipurpose application may recommend one or more available appointments to the computing device of the user for presentation.

The autonomous multipurpose application may enable a user to schedule numerous appointments for himself or herself with people having different specialties via a single user interface. For example, the specialties may include a medical doctor (physician), a dentist, an optometrist, a physician's assistant, a chiropractor, a behavioral specialist, a lab technician, a masseuse, a barber, an orthodontist, a dermatologist, and the like. Also, the autonomous multipurpose application may enable the user to schedule appointments for dependents (e.g., children, spouse, senior citizen, etc.) of an insurance plan.

Further, the autonomous multipurpose application may function as a centralized manager and repository for documents pertaining to the user and the dependents of the user. For example, when a user checks-in using a computing device (e.g., kiosk) executing the autonomous multipurpose application at a clinic, check-in documents pertaining to the user stored in a database may be checked to determine whether the check-in documents are complete. The check-in documents may refer to consent forms, medical history documents, health information release authorization forms, new patient sheets, massage client intake forms, mental health intake forms, consent treatment for minor child forms, doctor referral forms, adult health history forms, school physical forms, insurance verification sheets, medical reports, therapy intake forms, initial exam reports, pain assessment sheets, and the like. In some embodiments, the autonomous multipurpose application may communicate with external systems, such as EMR systems, to request the documents for the user from those systems. For example, if the user checked-in for another appointment with a different physician, the user may have already completed the various check-in documents and the autonomous multipurpose application may retrieve those completed check-in documents and store them for future reference. The autonomous multipurpose application may transmit the completed check-in documents to the EMR system associated with the person with which the user has an appointment.

If the check-in documents are partially complete, the autonomous multipurpose application may cause the portions of information that are missing to be presented for completion. If the check-in documents are incomplete, the autonomous multipurpose application may cause the check-in documents to be presented on a computing device for completion by the user, an administrator, a person having a specialty, or the like.

The autonomous multipurpose application may also manage and store other information for the users. For example, the user may capture an image of their driver's license, insurance card, and the like, and transmit the image to the autonomous multipurpose application. The autonomous multipurpose application may analyze the image (e.g., using machine learning and/or optical character recognition) to extract information from the image. For example, the autonomous multipurpose application may extract a picture of the user from a driver's license, a name of the user, a birthdate of the user, an address of the user, an identification number, an insurance plan number, a type of insurance, an expiration date of the user's driver's license, an expiration date of the user's insurance plan, and the like. The autonomous multipurpose application may electronically fill information in corresponding documents based on the extracted information. Further, the autonomous multipurpose application may perform logic based on the extracted information. For example, if the user's insurance is about to expire, the autonomous multipurpose application may transmit a message (e.g., email, text message, phone call, onscreen notification, etc.) to the user to renew their insurance. Similar types of information may be managed and stored for each person in a family. The information may be disbursed to a requesting client, such as an EMR system used by an entity at which the users make appointments.

The autonomous multipurpose application may communicate with a knowledge cloud that includes knowledge graphs that each pertain to a respective medical condition. For example, each knowledge graph may include individual elements (e.g., health artifacts) and predicates that describe relationships between the individual elements in a logical structure. Each knowledge graph may include nodes representing the individual elements and branches representing the predicates that connect the nodes. Each knowledge graph may begin at a root node that includes a type or name of the medical condition, for example. One knowledge graph may include a root node representing “Diabetes”. A predicate may represent “is caused by” branch that connects to another node “high blood sugar”. The logical structure may be formulated as “Diabetes is caused by high blood sugar”.

When a user successfully checks-in for a scheduled appointment, the autonomous multipurpose application may access the knowledge cloud to obtain curated content pertaining to one or more conditions of the user. For example, the user may specify the condition for which the user is seeking treatment, and educational curated content about that condition may be recommended and/or provided to the computing device of the user. The autonomous multipurpose application may also recommend other curated content to the user for the conditions of the user that are known by the autonomous multipurpose application. Each time a user has an appointment, the autonomous multipurpose application may update information pertaining to the user to keep knowledge about the user up to date.

In addition, when the user is checked-in, a wait time estimator model may be used by the autonomous multipurpose application to provide an estimated wait time. For example, the wait time estimator may be a machine learning model that is trained using data representing an average amount of time it takes a person having a specialty to perform a service. The training data may be specific for each different person and the amount of time it takes that person to perform the service. The wait time estimator may use training data pertaining to each patient. For example, if John Smith is at an appointment in the doctor's office immediately before Jane Doe, the average time that John Smith stays in the office may be used to estimate the wait time for Jane Doe. The wait times from different offices and/or clinics may be aggregated for each specialty in that office and/or for each person having the specialties to perform the service associated with the specialties.

Various timestamps associated with interactions between the user and the person having the specialty may be obtained from a system (e.g., EMR) used by the person having the specialty. For example, a timestamp of when the user checked-in for a scheduled appointment may be obtained, a timestamp of how long it took for the user to be called back to the doctor's office may be obtained, a timestamp of how long the user waited in the doctor's office prior to the doctor entering, a timestamp of any patient notes made by the doctor, a timestamp of any patient notes made by a nurse, a timestamp of when the doctor leaves after performing a service, a timestamp of when the user pays, or some combination thereof. The timestamps may be used to estimate wait times for users that have appointments scheduled with that doctor.

The autonomous multipurpose application may provide natural language searching for content. For example, the user may search “information about Diabetes” and the autonomous multipurpose application may return curated content pertaining to Diabetes to the computing device of the user.

The disclosed autonomous multipurpose application may provide an enhanced experience for users by improving scheduling, check-in, wait time estimation, cost transparency, and/or content distribution, among other things. The autonomous multipurpose application may use artificial intelligence to make decisions and perform actions.

The described methods and systems are described as occurring in the healthcare space, though other areas are also contemplated, such as finance, career, etc.

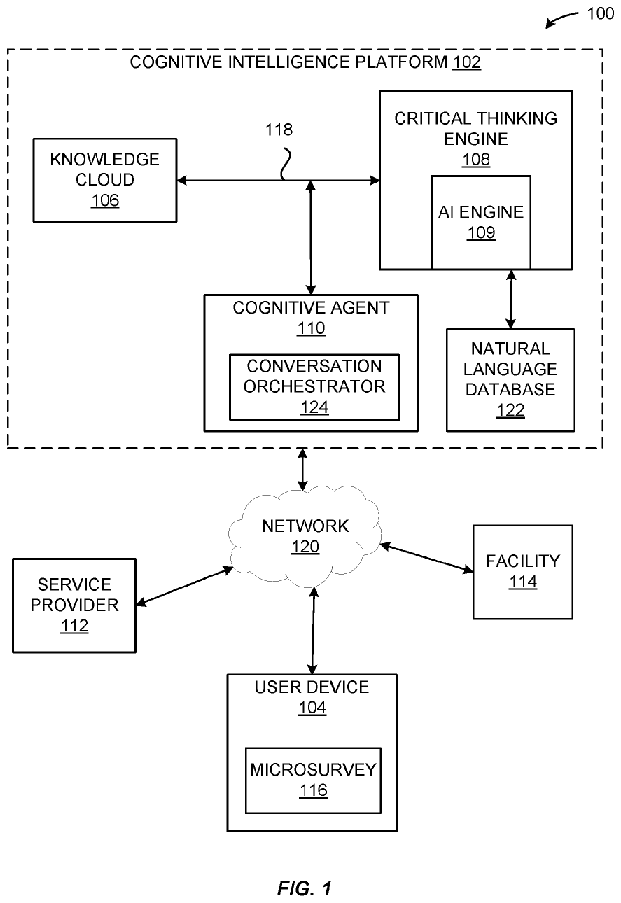

FIG. 1 shows a system architecture 100 that can be configured to provide a population health management service, in accordance with various embodiments. Specifically, FIG. 1 illustrates a high-level overview of an overall architecture that includes a cognitive intelligence platform 102 communicably coupled to a user device 104. The cognitive intelligence platform 102 includes several computing devices, where each computing device, respectively, includes at least one processor, at least one memory, and at least one storage (e.g., a hard drive, a solid-state storage device, a mass storage device, and a remote storage device). The individual computing devices can represent any form of a computing device such as a desktop computing device, a rack-mounted computing device, and a server device. The foregoing example computing devices are not meant to be limiting. On the contrary, individual computing devices implementing the cognitive intelligence platform 102 can represent any form of computing device without departing from the scope of this disclosure.

The several computing devices work in conjunction to implement components of the cognitive intelligence platform 102 including: a knowledge cloud 106; a critical thinking engine 108; a natural language database 122; and a cognitive agent 110. The cognitive intelligence platform 102 is not limited to implementing only these components, or in the manner described in FIG. 1. That is, other system architectures can be implemented, with different or additional components, without departing from the scope of this disclosure. The example system architecture 100 illustrates one way to implement the methods and techniques described herein.

The knowledge cloud 106 represents a set of instructions executing within the cognitive intelligence platform 102 that implement a database configured to receive inputs from several sources and entities. For example, some of the sources and entities include a service provider 112, a facility 114, and a microsurvey 116—each described further below.

The critical thinking engine 108 represents a set of instructions executing within the cognitive intelligence platform 102 that execute tasks using artificial intelligence, such as recognizing and interpreting natural language (e.g., performing conversational analysis), and making decisions in a linear manner (e.g., in a manner similar to how the human left brain processes information). Specifically, an ability of the cognitive intelligence platform 102 to understand natural language is powered by the critical thinking engine 108. In various embodiments, the critical thinking engine 108 includes a natural language database 122. The natural language database 122 includes data curated over at least thirty years by linguists and computer data scientists, including data related to speech patterns, speech equivalents, and algorithms directed to parsing sentence structure.

Furthermore, the critical thinking engine 108 is configured to deduce causal relationships given a particular set of data, where the critical thinking engine 108 is capable of taking the individual data in the particular set, arranging the individual data in a logical order, deducing a causal relationship between each of the data, and drawing a conclusion. The ability to deduce a causal relationship and draw a conclusion (referred to herein as a “causal” analysis) is in direct contrast to other implementations of artificial intelligence that mimic the human left brain processes. For example, the other implementations can take the individual data and analyze the data to deduce properties of the data or statistics associated with the data (referred to herein as an “analytical” analysis). However, these other implementations are unable to perform a causal analysis—that is, deduce a causal relationship and draw a conclusion from the particular set of data. As described further below—the critical thinking engine 108 is capable of performing both types of analysis: causal and analytical.

In some embodiments, the critical thinking engine 108 includes an artificial intelligence engine 109 (“Al Engine” in FIG. 1) that uses one or more machine learning models. The one or more machine learning models may be generated by a training engine and may be implemented in computer instructions that are executable by one or more processing device of the training engine, the artificial intelligence engine 109, another server, and/or the user device 104. To generate the one or more machine learning models, the training engine may train, test, and validate the one or more machine learning models. The training engine may be a rackmount server, a router computer, a personal computer, a portable digital assistant, a smartphone, a laptop computer, a tablet computer, a camera, a video camera, a netbook, a desktop computer, a media center, or any combination of the above. The one or more machine learning models may refer to model artifacts that are created by the training engine using training data that includes training inputs and corresponding target outputs. The training engine may find patterns in the training data that map the training input to the target output, and generate the machine learning models that capture these patterns.

The one or more machine learning models may be trained to generate one or more knowledge graphs each pertaining to a particular medical condition. The knowledge graphs may include individual elements (nodes) that are linked via predicates of a logical structure. The logical structure may use any suitable order of logic (e.g., higher order logic and/or Nth order logic). Higher order logic may be used to admit quantification over sets that are nested arbitrarily deep. Higher order logic may refer to a union of first-, second-, third, . . . , Nth order logic. Clinical-based evidence, clinical trials, physician research, and the like that includes various information (e.g., knowledge) pertaining to different medical conditions may be input as training data to the one or more machine learning models. The information may pertain to facts, properties, attributes, concepts, conclusions, risks, correlations, complications, etc. of the medical conditions. Keywords, phrases, sentences, cardinals, numbers, values, objectives, nouns, verbs, concepts, and so forth may be specified (e.g., labeled) in the information such that the machine learning models learn which ones are associated with the medical conditions. The information may specify predicates that correlates the information in a logical structure such that the machine learning models learn the logical structure associated with the medical conditions.

In some embodiments, the one or more machine learning models may be trained to transform input unstructured data (e.g., patient notes) into cognified data using the knowledge graph and the logical structure. The machine learning models may identify indicia in the unstructured data and compare the indicia to the knowledge graphs to generate possible health related information (e.g., tags) pertaining to the patient. The possible health related information may be associated with the indicia in the unstructured data. The one or more machine learning models may also identify, using the logical structure, a structural similarity of the possible health related information and a known predicate in the logical structure. The structural similarity between the possible health related information and the known predicate may enable identifying a pattern (e.g., treatment patterns, education and content patterns, order patterns, referral patterns, quality of care patterns, risk adjustment patterns, etc.). The one or more machine learning models may generate the cognified data based on the structural similarity and/or the pattern identified. Accordingly, the machine learning models may use a combination of knowledge graphs, logical structures, structural similarity comparison mechanisms, and/or pattern recognition to generate the cognified data. The cognified data may be output by the one or more trained machine learning models.

The cognified data may provide a summary of the medical condition of the patient. A diagnosis of the patient may be generated based on the cognified data. The summary of the medical condition may include one or more insights not present in the unstructured data. The summary may identify gaps in the unstructured data, such as treatment gaps (e.g., should prescribe medication, should provide different medication, should change dosage of medication, etc.), risk gaps (e.g., the patient is at risk for cancer based on familial history and certain lifestyle behaviors), quality of care gaps (e.g., need to check-in with the patient more frequently), and so forth. The summary of the medical condition may include one or more conclusions, recommendations, complications, risks, statements, causes, symptoms, etc. pertaining to the medical condition. In some embodiments, the summary of the medical condition may indicate another medical condition that the medical condition can lead to. Accordingly, the cognified data represents intelligence, knowledge, and logic cognified from unstructured data.

In some embodiments, the cognified data may be reviewed by physicians and the physicians may provide feedback pertaining to whether or not the cognified data is accurate. Also, the physicians may provide feedback pertaining to whether or not the diagnosis generated using the cognified data is accurate. This feedback may be used to update the one or more machine learning models to improve their accuracy.

The AI engine 109 may include machine learning models that are trained to schedule appointments for users, recommend appointments to users, determine costs of services, manage documents for users, extract data from images, provide curated content tailored for users, estimate wait times, perform natural language searching of curated content, and so forth.

The cognitive agent 110 represents a set of instructions executing within the cognitive intelligence platform 102 that implement a client-facing component of the cognitive intelligence platform 102. The cognitive agent 110 may be referred to as the autonomous multipurpose application interchangeably herein. The cognitive agent 110 is an interface between the cognitive intelligence platform 102 and the user device 104. And in some embodiments, the cognitive agent 110 includes a conversation orchestrator 124 that determines pieces of communication that are presented to the user device 104 (and the user). When a user of the user device 104 interacts with the cognitive intelligence platform 102, the user interacts with the cognitive agent 110. In some embodiments, the user of the user device 104 may be a patient. The several references herein, to the cognitive agent 110 performing a method, can implicate actions performed by the critical thinking engine 108, which accesses data in the knowledge cloud 106 and the natural language database 122.

Various user interfaces may be provided to computing devices communicating with the cognitive agent 110 executing in the cognitive intelligence platform 102. The user interfaces may be presented in a standalone application executing on the devices or in a web browser as website pages. In some embodiments, the cognitive agent 110 may be installed on a device of the user, the service provider 112, and/or the facility 114. In some embodiments, the devices of the user, the service provider 112, and/or the facility 114 may communicate with cognitive intelligence platform 102 in a client-server architecture. In some embodiments, the cognitive agent 110 may be implemented as computer instructions as an application programming interface.

In various embodiments, the several computing devices executing within the cognitive intelligence platform are communicably coupled by way of a network/bus interface. Furthermore, the various components (e.g., the knowledge cloud 106, the critical thinking engine 108, and the cognitive agent 110), are communicably coupled by one or more inter-host communication protocols 118. In one example, the knowledge cloud 106 is implemented using a first computing device, the critical thinking engine 108 is implemented using a second computing device, and the cognitive agent 110 is implemented using a third computing device, where each of the computing devices are coupled by way of the inter-host communication protocol 118. Although in this example, the individual components are described as executing on separate computing devices this example is not meant to be limiting, the components can be implemented on the same computing device, or partially on the same computing device, without departing from the scope of this disclosure.

The user device 104 represents any form of a computing device, or network of computing devices, e.g., a personal computing device, a smart phone, a tablet, a wearable computing device, a notebook computer, a media player device, and a desktop computing device. The user device 104 includes a processor, at least one memory, and at least one storage. A user uses the user device 104 to input a given text posed in natural language (e.g., typed on a physical keyboard, spoken into a microphone, typed on a touch screen, or combinations thereof) and interacts with the cognitive intelligence platform 102, by way of the cognitive agent 110.

The architecture 100 includes a network 120 that communicatively couples various devices, including the cognitive intelligence platform 102 and the user device 104. The network 120 can include local area network (LAN) and wide area networks (WAN). The network 102 can include wired technologies (e.g., Ethernet®) and wireless technologies (e.g., Wi-Fi®, code division multiple access (CDMA), global system for mobile (GSM), universal mobile telephone service (UMTS), Bluetooth®, and ZigBee®. For example, the user device 104 can use a wired connection or a wireless technology (e.g., Wi-Fi®) to transmit and receive data over the network 120.

Still referring to FIG. 1, the knowledge cloud 106 is configured to receive data from various sources and entities and integrate the data in a database. An example source that provides data to the knowledge cloud 106 is the service provider 112, an entity that provides a type of service to a user. For example, the service provider 112 can be a health service provider (e.g., a doctor's office, a physical therapist's office, a nurse's office, or a clinical social worker's office), and a financial service provider (e.g., an accountant's office). For purposes of this discussion, the cognitive intelligence platform 102 provides services in the health industry, thus the examples discussed herein are associated with the health industry. However, any service industry can benefit from the disclosure herein, and thus the examples associated with the health industry are not meant to be limiting.

Throughout the course of a relationship between the service provider 112 and a user (e.g., the service provider 112 provides healthcare to a patient), the service provider 112 collects and generates data associated with the patient or the user, including health records that include doctor's notes about the patient and prescriptions, billing records, and insurance records. The service provider 112, using a computing device (e.g., a desktop computer or a tablet), provides the data associated with the user to the cognitive intelligence platform 102, and more specifically the knowledge cloud 106.

Another example source that provides data to the knowledge cloud 106 is the facility 114. The facility 114 represents a location owned, operated, or associated with any entity including the service provider 112. As used herein, an entity represents an individual or a collective with a distinct and independent existence. An entity can be legally recognized (e.g., a sole proprietorship, a partnership, a corporation) or less formally recognized in a community. For example, the entity can include a company that owns or operates a gym (facility). Additional examples of the facility 114 include, but is not limited to, a hospital, a trauma center, a clinic, a dentist's office, a pharmacy, a store (including brick and mortar stores and online retailers), an out-patient care center, a specialized care center, a birthing center, a gym, a cafeteria, and a psychiatric care center.

As the facility 114 represents a large number of types of locations, for purposes of this discussion and to orient the reader by way of example, the facility 114 represents the doctor's office or a gym. The facility 114 generates additional data associated with the user such as appointment times, an attendance record (e.g., how often the user goes to the gym), a medical record, a billing record, a purchase record, an order history, and an insurance record. The facility 114, using a computing device (e.g., a desktop computer or a tablet), provides the data associated with the user to the cognitive intelligence platform 102, and more specifically the knowledge cloud 106.

An additional example source that provides data to the knowledge cloud 106 is the microsurvey 116. The microsurvey 116 represents a tool created by the cognitive intelligence platform 102 that enables the knowledge cloud 106 to collect additional data associated with the user. The microsurvey 116 is originally provided by the cognitive intelligence platform 102 (by way of the cognitive agent 110) and the user provides data responsive to the microsurvey 116 using the user device 104. Additional details of the microsurvey 116 are described below.

Yet another example source that provides data to the knowledge cloud 106, is the cognitive intelligence platform 102, itself In order to address the care needs and well-being of the user, the cognitive intelligence platform 102 collects, analyzes, and processes information from the user, healthcare providers, and other eco-system participants, and consolidates and integrates the information into knowledge. For example, clinical-based evidence and guidelines may be obtained by the cognitive intelligence platform 102 and used as knowledge. The knowledge can be shared with the user and stored in the knowledge cloud 106.

In various embodiments, the computing devices used by the service provider 112 and the facility 114 are communicatively coupled to the cognitive intelligence platform 102, by way of the network 120. While data is used individually by various entities including: a hospital, practice group, facility, or provider, the data is less frequently integrated and seamlessly shared between the various entities in the current art. The cognitive intelligence platform 102 provides a solution that integrates data from the various entities. That is, the cognitive intelligence platform 102 ingests, processes, and disseminates data and knowledge in an accessible fashion, where the reason for a particular answer or dissemination of data is accessible by a user.

In particular, the cognitive intelligence platform 102 (e.g., by way of the cognitive agent 110 interacting with the user) holistically manages and executes a health plan for durational care and wellness of the user (e.g., a patient or consumer). The health plan includes various aspects of durational management that is coordinated through a care continuum.

The cognitive agent 110 can implement various personas that are customizable. For example, the personas can include knowledgeable (sage), advocate (coach), and witty friend (jester). And in various embodiments, the cognitive agent 110 persists with a user across various interactions (e.g., conversations streams), instead of being transactional or transient. Thus, the cognitive agent 110 engages in dynamic conversations with the user, where the cognitive intelligence platform 102 continuously deciphers topics that a user wants to talk about. The cognitive intelligence platform 102 has relevant conversations with the user by ascertaining topics of interest from a given text posed in a natural language input by the user. Additionally the cognitive agent 110 connects the user to healthcare service providers, hyperlocal health communities, and a variety of services and tools/devices, based on an assessed interest of the user.

As the cognitive agent 110 persists with the user, the cognitive agent 110 can also act as a coach and advocate while delivering pieces of information to the user based on tonal knowledge, human-like empathies, and motivational dialog within a respective conversational stream, where the conversational stream is a technical discussion focused on a specific topic. Overall, in response to a question—e.g., posed by the user in natural language—the cognitive intelligence platform 102 consumes data from and related to the user and computes an answer. The answer is generated using a rationale that makes use of common sense knowledge, domain knowledge, evidence-based medicine guidelines, clinical ontologies, and curated medical advice. Thus, the content displayed by the cognitive intelligence platform 102 (by way of the cognitive agent 110) is customized based on the language used to communicate with the user, as well as factors such as a tone, goal, and depth of topic to be discussed.

Overall, the cognitive intelligence platform 102 is accessible to a user, a hospital system, and physician. Additionally, the cognitive intelligence platform 102 is accessible to paying entities interested in user behavior—e.g., the outcome of physician-consumer interactions in the context of disease or the progress of risk management. Additionally, entities that provides specialized services such as tests, therapies, and clinical processes that need risk based interactions can also receive filtered leads from the cognitive intelligence platform 102 for potential clients.

Conversational Analysis

In various embodiments, the cognitive intelligence platform 102 is configured to perform conversational analysis in a general setting. The topics covered in the general setting is driven by the combination of agents (e.g., cognitive agent 110) selected by a user. In some embodiments, the cognitive intelligence platform 102 uses conversational analysis to identify the intent of the user (e.g., find data, ask a question, search for facts, find references, and find products) and a respective micro-theory in which the intent is logical.

For example, the cognitive intelligence platform 102 applies conversational analysis to decode what the user is asking or stated, where the question or statement is in free form language (e.g., natural language). Prior to determining and sharing knowledge (e.g., with the user or the knowledge cloud 106), using conversational analysis, the cognitive intelligence platform 102 identifies an intent of the user and overall conversational focus.

The cognitive intelligence platform 102 responds to a statement or question according to the conversational focus and steers away from another detected conversational focus so as to focus on a goal defined by the cognitive agent 110. Given an example statement of a user, “I want to fly out tomorrow,” the cognitive intelligence platform 102 uses conversational analysis to determine an intent of the statement. Is the user aspiring to be bird-like or does he want to travel? In the former case, the micro-theory is that of human emotions whereas in the latter case, the micro-theory is the world of travel. Answers are provided to the statement depending on the micro-theory in which the intent logically falls.

The cognitive intelligence platform 102 utilize a combination of linguistics, artificial intelligence, and decision trees to decode what a user is asking or stating. The discussion includes methods and system design considerations and results from an existing embodiment. Additional details related to conversational analysis are discussed next.

Analyzing Conversational Context as Part of Conversational Analysis
For purposes of this discussion, the concept of analyzing conversational context as part of conversational analysis is now described. To analyze conversational context, the following steps are taken: 1) obtain text (e.g., receive a question) and perform translations; 2) understand concepts, entities, intents, and micro-theory; 3) relate and search; 4) ascertain the existence of related concepts; 5) logically frame concepts or needs; 6) understand the questions that can be answered from available data; and 7) answer the question. Each of the foregoing steps is discussed next, in turn.

Step 1: Obtain Text/Question and Perform Translations

In various embodiments, the cognitive intelligence platform 102 (FIG. 1) receives a text or question and performs translations as appropriate. The cognitive intelligence platform 102 supports various methods of input including text received from a touch interface (e.g., options presented in a microsurvey), text input through a microphone (e.g., words spoken into the user device), and text typed on a keyboard or on a graphical user interface. Additionally, the cognitive intelligence platform 102 supports multiple languages and auto translation (e.g., from English to Traditional/Simplified Chinese or vice versa).

The example text below is used to described methods in accordance with various embodiments herein:
“One day in January 1913, G. H. Hardy, a famous Cambridge University mathematician received a letter from an Indian named Srinivasa Ramanujan asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, though born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

The cognitive intelligence platform 102 analyzes the example text above to detect structural elements within the example text (e.g., paragraphs, sentences, and phrases). In some embodiments, the example text is compared to other sources of text such as dictionaries, and other general fact databases (e.g., Wikipedia) to detect synonyms and common phrases present within the example text.

Step 2: Understand Concept, Entity, Intent, and Micro-Theory

In step 2, the cognitive intelligence platform 102 parses the text to ascertain concepts, entities, intents, and micro-theories. An example output after the cognitive intelligence platform 102 initially parses the text is shown below, where concepts, and entities are shown in bold.
“One day in January 1913, G. H. Hardy, a famous Cambridge University mathematician received a letter from an Indian named Srinivasa Ramanujan asking him for his opinion of 120 mathematical theorems that Ramanujan said he had discovered. To Hardy, many of the theorems made no sense. Of the others, one or two were already well-known. Ramanujan must be some kind of trickplayer, Hardy decided, and put the letter aside. But all that day the letter kept hanging round Hardy. Might there by something in those wild-looking theorems?
That evening Hardy invited another brilliant Cambridge mathematician, J. E. Littlewood, and the two men set out to assess the Indian's worth. That incident was a turning point in the history of mathematics.
At the time, Ramanujan was an obscure Madras Port Trust clerk. A little more than a year later, he was at Cambridge University, and beginning to be recognized as one of the most amazing mathematicians the world has ever known. Though he died in 1920, much of his work was so far in advance of his time that only in recent years is it beginning to be properly understood.
Indeed, his results are helping solve today's problems in computer science and physics, problems that he could have had no notion of.
For Indians, moreover, Ramanujan has a special significance. Ramanujan, though born in poor and ill-paid accountant's family 100 years ago, has inspired many Indians to adopt mathematics as career.
Much of Ramanujan's work is in number theory, a branch of mathematics that deals with the subtle laws and relationships that govern numbers. Mathematicians describe his results as elegant and beautiful but they are much too complex to be appreciated by laymen.
His life, though, is full of drama and sorrow. It is one of the great romantic stories of mathematics, a distressing reminder that genius can surface and rise in the most unpromising circumstances.”

For example, the cognitive intelligence platform 102 ascertains that Cambridge is a university—which is a full understanding of the concept. The cognitive intelligence platform (e.g., the cognitive agent 110) understands what humans do in Cambridge, and an example is described below in which the cognitive intelligence platform 102 performs steps to understand a concept.

For example, in the context of the above example, the cognitive agent 110 understands the following concepts and relationships:

Cambridge employed John Edensor Littlewood (1)

Cambridge has the position Ramanujan's position at Cambridge University (2)

Cambridge employed G. H. Hardy. (3)

The cognitive agent 110 also assimilates other understandings to enhance the concepts, such as:

Cambridge has Trinity College as a suborganization. (4)

Cambridge is located in Cambridge. (5)

Alan Turing is previously enrolled at Cambridge. (6)

Stephen Hawking attended Cambridge. (7)

The statements (1)-(7) are not picked at random. Instead the cognitive agent 110 dynamically constructs the statements (1)-(7) from logic or logical inferences based on the example text above. Formally, the example statements (1)-(7) are captured as follows:

(#$subOrganizations #$UniversityOfCambridge #$TrinityCollege-Cambridge-England) (8)

(#$placeInCity #$UniversityOfCambridge #$Cityof CambridgeEngland) (9)

(#$schooling #$AlanTuring #$UniversityOfCambridge #$PreviouslyEnrolled) (10)

(#$hasAlumni #$UniversityOfCambridge #$StephenHawking) (11)

Step 3: Relate and Search

Next, in step 3, the cognitive agent 110 relates various entities and topics and follows the progression of topics in the example text. Relating includes the cognitive agent 110 understanding the different instances of Hardy are all the same person, and the instances of Hardy are different from the instances of Littlewood. The cognitive agent 110 also understands that the instances Hardy and Littlewood share some similarities—e.g., both are mathematicians and they did some work together at Cambridge on Number Theory. The ability to track this across the example text is referred to as following the topic progression with a context.

Step 4: Ascertain the Existence of Related Concepts

Next, in Step 4, the cognitive agent 110 asserts non-existent concepts or relations to form new knowledge. Step 4 is an optional step for analyzing conversational context. Step 4 enhances the degree to which relationships are understood or different parts of the example text are understood together. If two concepts appear to be separate—e.g., a relationship cannot be graphically drawn or logically expressed between enough sets of concepts—there is a barrier to understanding. The barriers are overcome by expressing additional relationships. The additional relationships can be discovered using strategies like adding common sense or general knowledge sources (e.g., using the common sense data 208) or adding in other sources including a lexical variant database, a dictionary, and a thesaurus.

One example of concept progression from the example text is as follows: the cognitive agent 110 ascertains the phrase “theorems that Ramanujan said he had discovered” is related to the phrase “his results”, which is related to “Ramanujan's work is in number theory, a branch of mathematics that deals with the subtle laws and relationships that govern numbers.”

Step 5: Logically Frame Concepts or Needs

In Step 5, the cognitive agent 110 determines missing parameters—which can include for example, missing entities, missing elements, and missing nodes—in the logical framework (e.g., with a respective micro-theory). The cognitive agent 110 determines sources of data that can inform the missing parameters. Step 5 can also include the cognitive agent 110 adding common sense reasoning and finding logical paths to solutions.

With regards to the example text, some common sense concepts include:

Mathematicians develop Theorems. (12)

Theorems are hard to comprehend. (13)

Interpretations are not apparent for years. (14)

Applications are developed over time. (15)

Mathematicians collaborate and assess work. (16)

With regards to the example text, some passage concepts include:

Ramanujan did Theorems in Early 20^{th} Century. (17)

Hardy assessed Ramanujan's Theorems. (18)

Hardy collaborated with Littlewood. (19)

Hardy and Littlewood assessed Ramanujan's work (20)

Within the micro-theory of the passage analysis, the cognitive agent 110 understands and catalogs available paths to answer questions. In Step 5, the cognitive agent 110 makes the case that the concepts (12)-(20) are expressed together.

Step 6: Understand the Questions That Can be Answered from Available Data

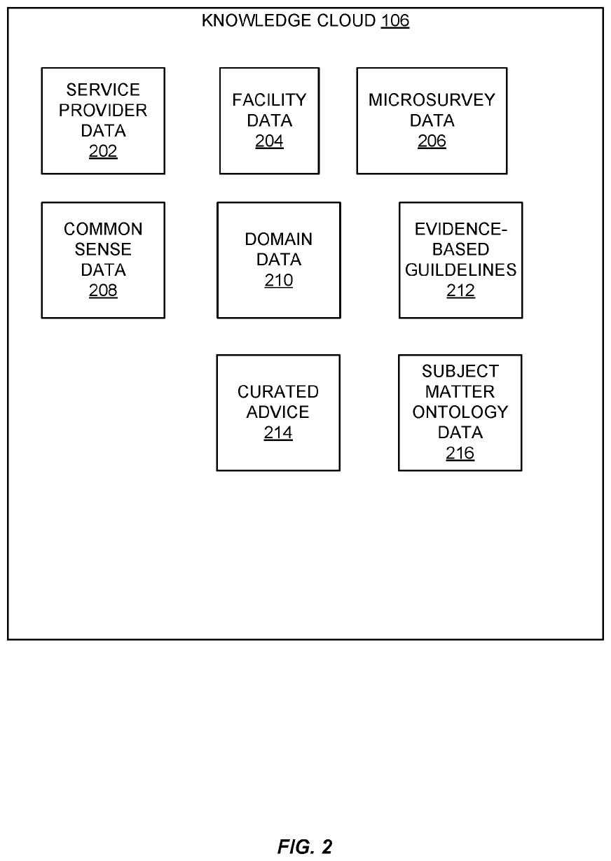

In Step 6, the cognitive agent 110 parses sub-intents and entities. Given the example text, the following questions are answerable from the cognitive agent's developed understanding of the example text, where the understanding was developed using information and context ascertained from the example text as well as the common sense data 208 (FIG. 2):

What situation causally contributed to Ramanujan's position at Cambridge? (21)

Does the author of the passage regret that Ramanujan died prematurely? (22)

Does the author of the passage believe that Ramanujan is a mathematical genius? (23)

Based on the information that is understood by the cognitive agent 110, the questions (21)-(23) can be answered.

By using an exploration method such as random walks, the cognitive agent 110 makes a determination as the paths that are plausible and reachable with the context (e.g., micro-theory) of the example text. Upon explorations, the cognitive agent 110 catalogs a set of meaningful questions. The set of meaningful questions are not asked, but instead explored based on the cognitive agent's understanding of the example text.

Given the example text, an example of exploration that yields a positive result is: “a situation X that caused Ramanujan's position.” In contrast, an example of exploration that causes irrelevant results is: “a situation Y that caused Cambridge.” The cognitive agent 110 is able to deduce that the latter exploration is meaningless, in the context of a micro-theory, because situations do not cause universities. Thus the cognitive agent 110 is able to deduce, there are no answers to Y, but there are answers to X.

Step 7: Answer the Question

In Step 7, the cognitive agent 110 provides a precise answer to a question. For an example question such as: “What situation causally contributed to Ramanujan's position at Cambridge?” the cognitive agent 110 generates a precise answer using the example reasoning:

HardyandLittlewoodsEvaluatingOfRamanujansWork (24)

HardyBeliefThatRamanujanIsAnExpertInMathematics (25)

HardysBeliefThatRamanujanIsAnExpertInMathematicsAndAGenius (26)

In order to generate the above reasoning statements (24)-(26), the cognitive agent 110 utilizes a solver or prover in the context of the example text's micro-theory—and associated facts, logical entities, relations, and assertions. As an additional example, the cognitive agent 110 uses a reasoning library that is optimized for drawing the example conclusions above within the fact, knowledge, and inference space (e.g., work space) that the cognitive agent 110 maintains.

By implementing the steps 1-7, the cognitive agent 110 analyzes conversational context. The described method for analyzing conversation context can also be used for recommending items in conversations streams. A conversational stream is defined herein as a technical discussion focused on specific topics. As related to described examples herein, the specific topics relate to health (e.g., diabetes). Throughout the lifetime of a conversational stream, a cognitive agent 110 collect information over may channels such as chat, voice, specialized applications, web browsers, contact centers, and the like.

By implementing the methods to analyze conversational context, the cognitive agent 110 can recommend a variety of topics and items throughout the lifetime of the conversational stream. Examples of items that can be recommended by the cognitive agent 110 include: surveys, topics of interest, local events, devices or gadgets, dynamically adapted health assessments, nutritional tips, reminders from a health events calendar, and the like.

Accordingly, the cognitive intelligence platform 102 provides a platform that codifies and takes into consideration a set of allowed actions and a set of desired outcomes. The cognitive intelligence platform 102 relates actions, the sequences of subsequent actions (and reactions), desired sub-outcomes, and outcomes, in a way that is transparent and logical (e.g., explainable). The cognitive intelligence platform 102 can plot a next best action sequence and a planning basis (e.g., health care plan template, or a financial goal achievement template), also in a manner that is explainable. The cognitive intelligence platform 102 can utilize a critical thinking engine 108 and a natural language database 122 (e.g., a linguistics and natural language understanding system) to relate conversation material to actions.

For purposes of this discussion, several examples are discussed in which conversational analysis is applied within the field of durational and whole-health management for a user. The discussed embodiments holistically address the care needs and well-being of the user during the course of his life. The methods and systems described herein can also be used in fields outside of whole-health management, including: phone companies that benefits from a cognitive agent; hospital systems or physicians groups that want to coach and educate patients; entities interested in user behavior and the outcome of physician-consumer interactions in terms of a progress of disease or risk management; entities that provide specialized services (e.g., test, therapies, clinical processes) to filter leads; and sellers, merchants, stores and big box retailers that want to understand which product to sell.

In addition, the conversational analysis may include cognifying the text input by the user. For example, if the user states (e.g., text, voice) they have various symptoms, the cognification techniques disclosed herein may be performed to construct cognified data using the text input. The user may input text specifying that they have a level of 5.7 mmol/L blood sugar. The cognitive intelligence platform 102 may cognify the text to output that the level of blood sugar is within acceptable limits, and that blood sugar testing was used to measure the blood sugar level. In some embodiments, the cognification techniques may be performed to generate a diagnosis of a medical condition of the patient. Further, the cognitive intelligence platform 102 may provide information to the user pertaining to the medical condition at a regulated pace.

FIG. 2 shows additional details of a knowledge cloud, in accordance with various embodiments. In particular, FIG. 2 illustrates various types of data received from various sources, including service provider data 202, facility data 204, microsurvey data 206, commonsense data 208, domain data 210, evidence-based guidelines 212, subject matter ontology data 214, and curated advice 216. The types of data represented by the service provider data 202 and the facility data 204 include any type of data generated by the service provider 112 and the facility 114, and the above examples are not meant to be limiting. Thus, the example types of data are not meant to be limiting and other types of data can also be stored within the knowledge cloud 106 without departing from the scope of this disclosure.

The service provider data 202 is data provided by the service provider 112 (described in FIG. 1) and the facility data 204 is data provided by the facility 114 (described in FIG. 1). For example, the service provider data 202 includes medical records of a respective patient of a service provider 112 that is a doctor. In another example, the facility data 204 includes an attendance record of the respective patient, where the facility 114 is a gym. The microsurvey data 206 is data provided by the user device 104 responsive to questions presented in the microsurvey 116 (FIG. 1).

Common sense data 208 is data that has been identified as “common sense”, and can include rules that govern a respective concept and used as glue to understand other concepts.

Domain data 210 is data that is specific to a certain domain or subject area. The source of the domain data 210 can include digital libraries. In the healthcare industry, for example, the domain data 210 can include data specific to the various specialties within healthcare such as, obstetrics, anesthesiology, and dermatology, to name a few examples. In the example described herein, the evidence-based guidelines 212 include systematically developed statements to assist practitioner and patient decisions about appropriate health care for specific clinical circumstances.

Curated advice 214 includes advice from experts in a subject matter. The curated advice 214 can include peer-reviewed subject matter, and expert opinions. Subject matter ontology data 216 includes a set of concepts and categories in a subject matter or domain, where the set of concepts and categories capture properties and relationships between the concepts and categories.

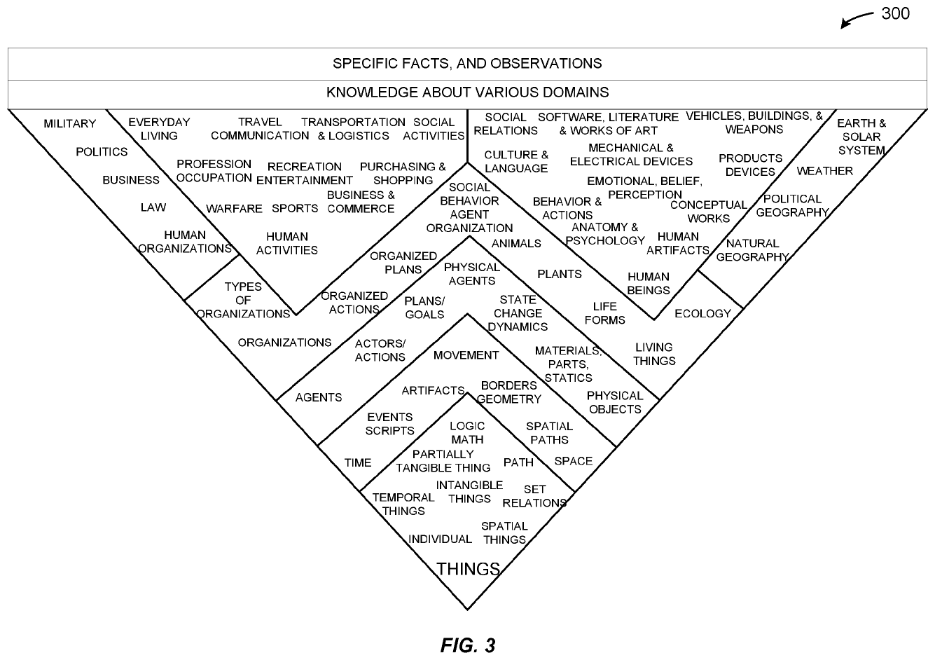

In particular, FIG. 3 illustrates an example subject matter ontology 300 that is included as part of the subject matter ontology data 216.

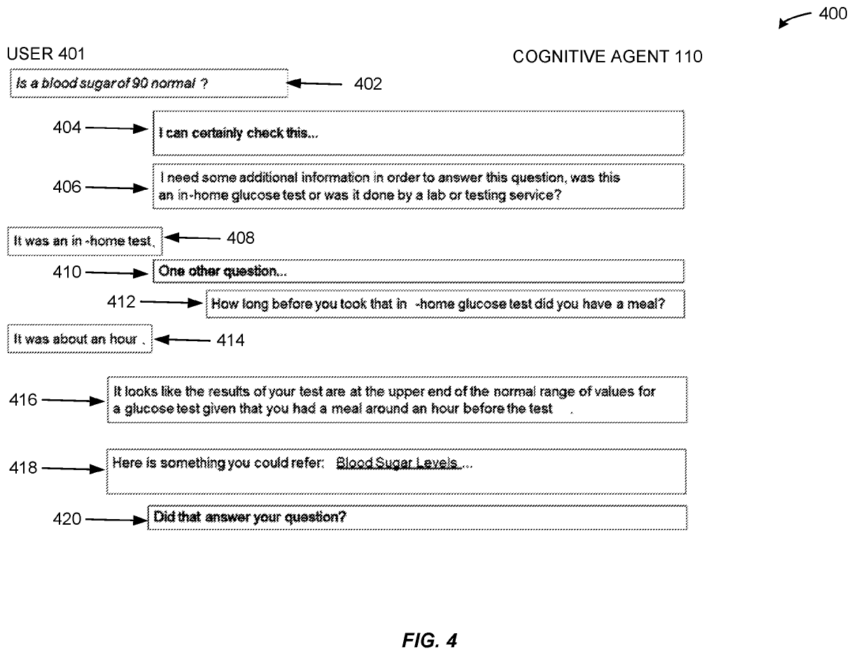

FIG. 4 illustrates aspects of a conversation 400 between a user and the cognitive intelligence platform 102, and more specifically the cognitive agent 110. For purposes of this discussion, the user 401 is a patient of the service provider 112. The user interacts with the cognitive agent 110 using a computing device, a smart phone, or any other device configured to communicate with the cognitive agent 110 (e.g., the user device 104 in FIG. 1). The user can enter text into the device using any known means of input including a keyboard, a touchscreen, and a microphone. The conversation 400 represents an example graphical user interface (GUI) presented to the user 401 on a screen of his computing device.

Initially, the user asks a general question, which is treated by the cognitive agent 110 as an “originating question.” The originating question is classified into any number of potential questions (“pursuable questions”) that are pursued during the course of a subsequent conversation. In some embodiments, the pursuable questions are identified based on a subject matter domain or goal. In some embodiments, classification techniques are used to analyze language (e.g., such as those outlined in HPS ID20180901-01_method for conversational analysis). Any known text classification technique can be used to analyze language and the originating question. For example, in line 402, the user enters an originating question about a subject matter (e.g., blood sugar) such as: “Is a blood sugar of 90 normal”? I

In response to receiving an originating question, the cognitive intelligence platform 102 (e.g., the cognitive agent 110 operating in conjunction with the critical thinking engine 108) performs a first round of analysis (e.g., which includes conversational analysis) of the originating question and, in response to the first round of analysis, creates a workspace and determines a first set of follow up questions.

In various embodiments, the cognitive agent 110 may go through several rounds of analysis executing within the workspace, where a round of analysis includes: identifying parameters, retrieving answers, and consolidating the answers. The created workspace can represent a space where the cognitive agent 110 gathers data and information during the processes of answering the originating question. In various embodiments, each originating question corresponds to a respective workspace. The conversation orchestrator 124 can assess data present within the workspace and query the cognitive agent 110 to determine if additional data or analysis should be performed.

In particular, the first round of analysis is performed at different levels, including analyzing natural language of the text, and analyzing what specifically is being asked about the subject matter (e.g., analyzing conversational context). The first round of analysis is not based solely on a subject matter category within which the originating question is classified. For example, the cognitive intelligence platform 102 does not simply retrieve a predefined list of questions in response to a question that falls within a particular subject matter, e.g., blood sugar. That is, the cognitive intelligence platform 102 does not provide the same list of questions for all questions related to the particular subject matter. Instead, for example, the cognitive intelligence platform 102 creates dynamically formulated questions, curated based on the first round of analysis of the originating question.

In particular, during the first round of analysis, the cognitive agent 110 parses aspects of the originating question into associated parameters. The parameters represent variables useful for answering the originating question. For example, the question “is a blood sugar of 90 normal” may be parsed and associated parameters may include, an age of the inquirer, the source of the value 90 (e.g., in home test or a clinical test), a weight of the inquirer, and a digestive state of the user when the test was taken (e.g., fasting or recently eaten). The parameters identify possible variables that can impact, inform, or direct an answer to the originating question.

For purposes of the example illustrated in FIG. 4, in the first round of analysis, the cognitive intelligence platform 102 inserts each parameter into the workspace associated with the originating question (line 402). Additionally, based on the identified parameters, the cognitive intelligence platform 102 identifies a customized set of follow up questions (“a first set of follow-up questions). The cognitive intelligence platform 102 inserts first set of follow-up questions in the workspace associated with the originating question.

The follow up questions are based on the identified parameters, which in turn are based on the specifics of the originating question (e.g., related to an identified micro-theory). Thus the first set of follow-up questions identified in response to, if a blood sugar is normal, will be different from a second set of follow up questions identified in response to a question about how to maintain a steady blood sugar.

After identifying the first set of follow up questions, in this example first round of analysis, the cognitive intelligence platform 102 determines which follow up question can be answered using available data and which follow-up question to present to the user. As described over the next few paragraphs, eventually, the first set of follow-up questions is reduced to a subset (“a second set of follow-up questions”) that includes the follow-up questions to present to the user.

In various embodiments, available data is sourced from various locations, including a user account, the knowledge cloud 106, and other sources. Other sources can include a service that supplies identifying information of the user, where the information can include demographics or other characteristics of the user (e.g., a medical condition, a lifestyle). For example, the service can include a doctor's office or a physical therapist's office.

Another example of available data includes the user account. For example, the cognitive intelligence platform 102 determines if the user asking the originating question, is identified. A user can be identified if the user is logged into an account associated with the cognitive intelligence platform 102. User information from the account is a source of available data. The available data is inserted into the workspace of the cognitive agent 110 as a first data.

Another example of available data includes the data stored within the knowledge cloud 106. For example, the available data includes the service provider data 202 (FIG. 2), the facility data 204, the microsurvey data 206, the common sense data 208, the domain data 210, the evidence-based guidelines 212, the curated advice 214, and the subject matter ontology data 216. Additionally data stored within the knowledge cloud 106 includes data generated by the cognitive intelligence platform 102, itself

Follow up questions presented to the user (the second set of follow-up questions) are asked using natural language and are specifically formulated (“dynamically formulated question”) to elicit a response that will inform or fulfill an identified parameter. Each dynamically formulated question can target one parameter at a time. When answers are received from the user in response to a dynamically formulated question, the cognitive intelligence platform 102 inserts the answer into the workspace. In some embodiments, each of the answers received from the user and in response to a dynamically formulated question, is stored in a list of facts. Thus the list of facts include information specifically received from the user, and the list of facts is referred to herein as the second data.

With regards to the second set of follow-up questions (or any set of follow-up questions), the cognitive intelligence platform 102 calculates a relevance index, where the relevance index provides a ranking of the questions in the second set of follow-up questions. The ranking provides values indicative of how relevant a respective follow-up question is to the originating question. To calculate the relevance index, the cognitive intelligence platform 102 can use conversations analysis techniques described in HPS ID20180901-01 method. In some embodiments, the first set or second set of follow up questions is presented to the user in the form of the microsurvey 116.

In this first round of analysis, the cognitive intelligence platform 102 consolidates the first and second data in the workspace and determines if additional parameters need to be identified, or if sufficient information is present in the workspace to answer the originating question. In some embodiments, the cognitive agent 110 (FIG. 1) assesses the data in the workspace and queries the cognitive agent 110 to determine if the cognitive agent 110 needs more data in order to answer the originating question. The conversation orchestrator 124 executes as an interface

For a complex originating question, the cognitive intelligence platform 102 can go through several rounds of analysis. For example, in a first round of analysis the cognitive intelligence platform 102 parses the originating question. In a subsequent round of analysis, the cognitive intelligence platform 102 can create a sub question, which is subsequently parsed into parameters in the subsequent round of analysis. The cognitive intelligence platform 102 is smart enough to figure out when all information is present to answer an originating question without explicitly programming or pre-programming the sequence of parameters that need to be asked about.

In some embodiments, the cognitive agent 110 is configured to process two or more conflicting pieces of information or streams of logic. That is, the cognitive agent 110, for a given originating question can create a first chain of logic and a second chain of logic that leads to different answers. The cognitive agent 110 has the capability to assess each chain of logic and provide only one answer. That is, the cognitive agent 110 has the ability to process conflicting information received during a round of analysis.

Additionally, at any given time, the cognitive agent 110 has the ability to share its reasoning (chain of logic) to the user. If the user does not agree with an aspect of the reasoning, the user can provide that feedback which results in affecting change in a way the critical thinking engine 108 analyzed future questions and problems.

Subsequent to determining enough information is present in the workspace to answer the originating question, the cognitive agent 110 answers the question, and additionally can suggest a recommendation or a recommendation (e.g., line 418). The cognitive agent 110 suggests the reference or the recommendation based on the context and questions being discussed in the conversation (e.g., conversation 400). The reference or recommendation serves as additional handout material to the user and is provided for informational purposes. The reference or recommendation often educates the user about the overall topic related to the originating question.

In the example illustrated in FIG. 4, in response to receiving the originating questions (line 402), the cognitive intelligence platform 102 (e.g., the cognitive agent 110 in conjunction with the critical thinking engine 108) parses the originating question to determine at least one parameter: location. The cognitive intelligence platform 102 categorizes this parameter, and a corresponding dynamically formulated question in the second set of follow-up questions. Accordingly, in lines 404 and 406, the cognitive agent 110 responds by notifying the user “I can certainly check this . . . ” and asking the dynamically formulated question “I need some additional information in order to answer this question, was this an in-home glucose test or was it done by a lab or testing service?”

The user 401 enters his answer in line 408: “It was an in-home test,” which the cognitive agent 110 further analyzes to determine additional parameters: e.g., a digestive state, where the additional parameter and a corresponding dynamically formulated question as an additional second set of follow-up questions. Accordingly, the cognitive agent 110 poses the additional dynamically formulated question in lines 410 and 412: “One other question . . . ” and “How long before you took that in-home glucose test did you have a meal?” The user provides additional information in response “it was about an hour” (line 414).

The cognitive agent 110 consolidates all the received responses using the critical thinking engine 108 and the knowledge cloud 106 and determines an answer to the initial question posed in line 402 and proceeds to follow up with a final question to verify the user's initial question was answered. For example, in line 416, the cognitive agent 110 responds: “It looks like the results of your test are at the upper end of the normal range of values for a glucose test given that you had a meal around an hour before the test.” The cognitive agent 110 provides additional information (e.g., provided as a link): “Here is something you could refer,” (line 418), and follows up with a question “Did that answer your question?” (line 420).

As described above, due to the natural language database 108, in various embodiments, the cognitive agent 110 is able to analyze and respond to questions and statements made by a user 401 in natural language. That is, the user 401 is not restricted to using certain phrases in order for the cognitive agent 110 to understand what a user 401 is saying. Any phrasing, similar to how the user would speak naturally can be input by the user and the cognitive agent 110 has the ability to understand the user.

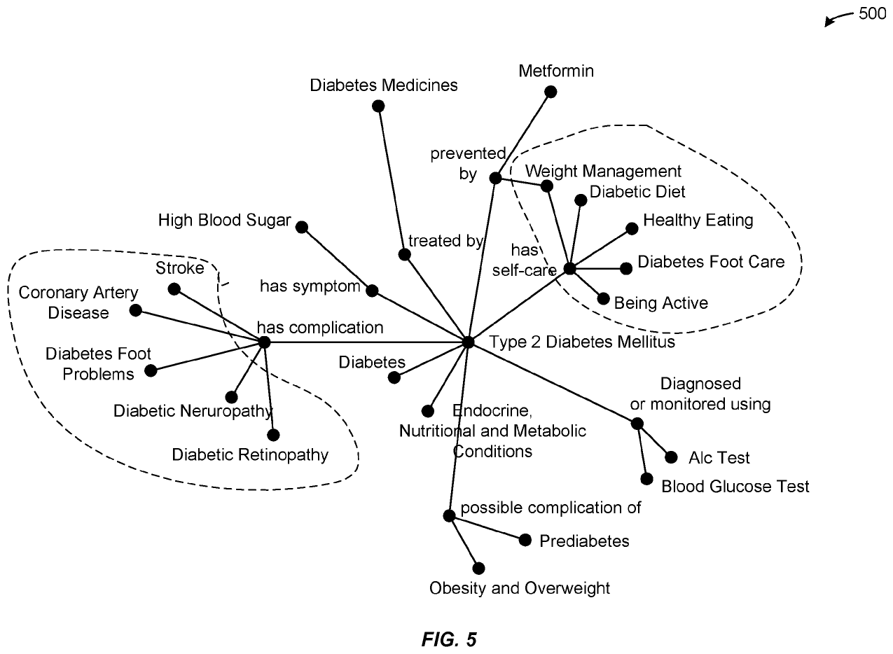

FIG. 5 illustrates a cognitive map or “knowledge graph” 500, in accordance with various embodiments. In particular, the knowledge graph represents a graph traversed by the cognitive intelligence platform 102, when assessing questions from a user with Type 2 diabetes. Individual nodes in the knowledge graph 500 represent a health artifact (health related information) or relationship (predicate) that is gleaned from direct interrogation or indirect interactions with the user (by way of the user device 104).

In one embodiment, the cognitive intelligence platform 102 identified parameters for an originating question based on a knowledge graph illustrated in FIG. 5. For example, the cognitive intelligence platform 102 parses the originating question to determine which parameters are present for the originating question. In some embodiments, the cognitive intelligence platform 102 infers the logical structure of the parameters by traversing the knowledge graph 500, and additionally, knowing the logical structure enables the cognitive agent 110 to formulate an explanation as to why the cognitive agent 110 is asking a particular dynamically formulated question.

In some embodiments, the individual elements or nodes are generated by the artificial intelligence engine based on input data (e.g., evidence-based guidelines, patient notes, clinical trials, physician research or the like). The artificial intelligence engine may parse the input data and construct the relationships between the health artifacts.

For example, a root node may be associated with a first health related information “Type 2 Diabetes Mellitus”, which is a name of a medical condition. In some embodiments, the root node may also be associated with a definition of the medical condition. An example predicate, “has symptom”, is represented by an individual node connected to the root node, and another health related information, “High Blood Sugar”, is represented by an individual node connected to the individual node representing the predicate. A logical structure may be represented by these three nodes, and the logical structure may indicate that “Type 2 Diabetes Mellitus has symptom High Blood Sugar”.

In some embodiments, the health related information may correspond to known facts, concepts, and/or any suitable health related information that are discovered or provided by a trusted source (e.g., a physician having a medical license and/or a certified/accredited healthcare organization), such as evidence-based guidelines, clinical trials, physician research, patient notes entered by physicians, and the like. The predicates may be part of a logical structure (e.g., sentence) such as a form of subject-predicate-direct object, subject-predicate-indirect object-direct object, subject-predicate-subject complement, or any suitable simple, compound, complex, and/or compound/complex logical structure. The subject may be a person, place, thing, health artifact, etc. The predicate may express an action or being within the logical structure and may be a verb, modifying words, phrases, and/or clauses. For example, one logical structure may be the subject-predicate-direct object form, such as “A has B” (where A is the subject and may be a noun or a health artifact, “has” is the predicate, and B is the direct object and may be a health artifact).

The various logical structures in the depicted knowledge graph may include the following: “Type 2 Diabetes Mellitus has symptom High Blood Sugar”; “Type 2 Diabetes Mellitus has complication Stroke”; “Type 2 Diabetes Mellitus has complication Coronary Artery Disease”; “Type 2 Diabetes Mellitus has complication Diabetes Foot Problems”; “Type 2 Diabetes Mellitus has complication Diabetic Neuropathy”; “Type 2 Diabetes Mellitus has complication Diabetic Retinopathy”; “Type 2 Diabetes Mellitus diagnosed or monitored using Blood Glucose Test”; just to name a few examples. It should be understood that there are other logical structures and represented in the knowledge graph 500.

In some embodiments, the information depicted in the knowledge graph may be represented as a matrix. The health artifacts may be represented as quantities and the predicates may be represented as expressions in a rectangular array in rows and columns of the matrix. The matrix may be treated as a single entity and manipulated according to particular rules.

The knowledge graph 500 or the matrix may be generated for each known medical condition and stored by the cognitive intelligence platform 102. The knowledge graphs and/or matrices may be updated continuously or on a periodic basis using subject data pertaining to the medical conditions received from the trusted sources. For example, additional clinical trials may lead to new discoveries about particular medical condition treatments, which may be used to update the knowledge graphs and/or matrices.

The knowledge graph 500 including the logical structures may be used to transform unstructured data (patient notes in an EMR entered by a physician) into cognified data. The cognified data may be used to generate a diagnosis of the patient. Also, the cognified data may be used to determine which information pertaining to the medical condition to provide to the patient and when to provide the information to the patient to improve the user experience using the computing device. The disclosed techniques may also save computing resources by providing the cognified data to the physician to review, improve diagnosis accuracy, and/or regulate the amount of information provided to the patient.

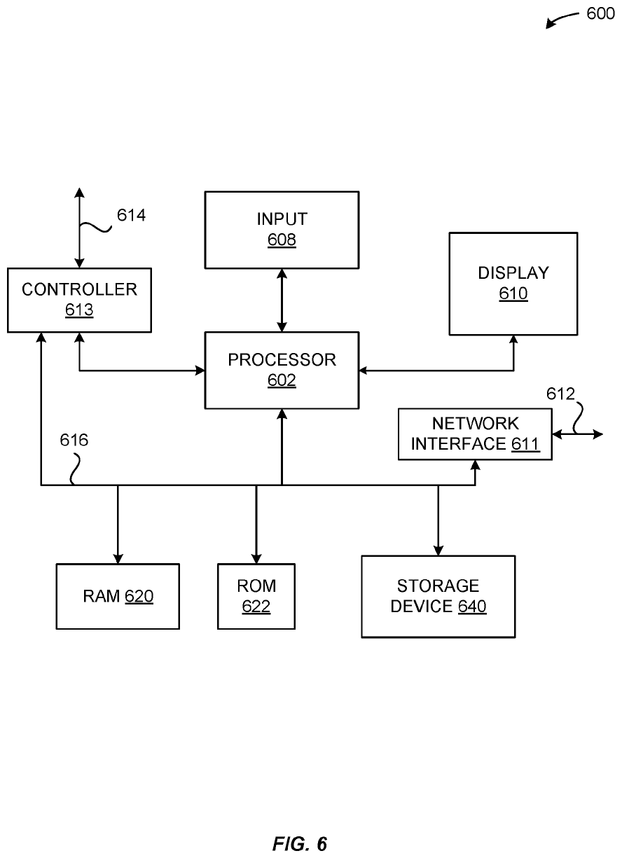

FIG. 6 illustrates a detailed view of a computing device 600 that can be used to implement the various components described herein, according to some embodiments. In particular, the detailed view illustrates various components that can be included in the user device 104 illustrated in FIG. 1, as well as the several computing devices implementing the cognitive intelligence platform 102. As shown in FIG. 6, the computing device 600 can include a processor 1402 that represents a microprocessor or controller for controlling the overall operation of the computing device 600. The computing device 600 can also include a user input device 1408 that allows a user of the computing device 600 to interact with the computing device 600. For example, the user input device 1408 can take a variety of forms, such as a button, keypad, dial, touch screen, audio input interface, visual/image capture input interface, input in the form of sensor data, and so on. Still further, the computing device 600 can include a display 1410 that can be controlled by the processor 1402 to display information to the user. A data bus 1416 can facilitate data transfer between at least a storage device 1440, the processor 1402, and a controller 1413. The controller 1413 can be used to interface with and control different equipment through an equipment control bus 1414. The computing device 600 can also include a network/bus interface 1411 that couples to a data link 1412. In the case of a wireless connection, the network/bus interface 1411 can include a wireless transceiver.

As noted above, the computing device 600 also includes the storage device 1440, which can comprise a single disk or a collection of disks (e.g., hard drives), and includes a storage management module that manages one or more partitions within the storage device 1440. In some embodiments, storage device 1440 can include flash memory, semiconductor (solid-state) memory or the like. The computing device 600 can also include a Random-Access Memory (RAM) 1420 and a Read-Only Memory (ROM) 1422. The ROM 1422 can store programs, utilities or processes to be executed in a non-volatile manner. The RAM 1420 can provide volatile data storage, and stores instructions related to the operation of processes and applications executing on the computing device.

FIG. 6 illustrates a detailed view of a computing device 600 that can be used to implement the various components described herein, according to some embodiments. In particular, the detailed view illustrates various components that can be included in the user device 104 illustrated in FIG. 1, as well as the several computing devices implementing the cognitive intelligence platform 102. As shown in FIG. 6, the computing device 600 can include a processor 602 that represents a microprocessor or controller for controlling the overall operation of the computing device 600. The computing device 600 can also include a user input device 608 that allows a user of the computing device 600 to interact with the computing device 600. For example, the user input device 608 can take a variety of forms, such as a button, keypad, dial, touch screen, audio input interface, visual/image capture input interface, input in the form of sensor data, and so on. Still further, the computing device 600 can include a display 610 that can be controlled by the processor 602 to display information to the user. A data bus 616 can facilitate data transfer between at least a storage device 640, the processor 602, and a controller 613. The controller 613 can be used to interface with and control different equipment through an equipment control bus 614. The computing device 600 can also include a network/bus interface 611 that couples to a data link 612. In the case of a wireless connection, the network/bus interface 611 can include a wireless transceiver.

As noted above, the computing device 600 also includes the storage device 640, which can comprise a single disk or a collection of disks (e.g., hard drives), and includes a storage management module that manages one or more partitions within the storage device 640. In some embodiments, storage device 640 can include flash memory, semiconductor (solid-state) memory or the like. The computing device 600 can also include a Random-Access Memory (RAM) 620 and a Read-Only Memory (ROM) 622. The ROM 622 can store programs, utilities or processes to be executed in a non-volatile manner. The RAM 620 can provide volatile data storage, and stores instructions related to the operation of processes and applications executing on the computing device.

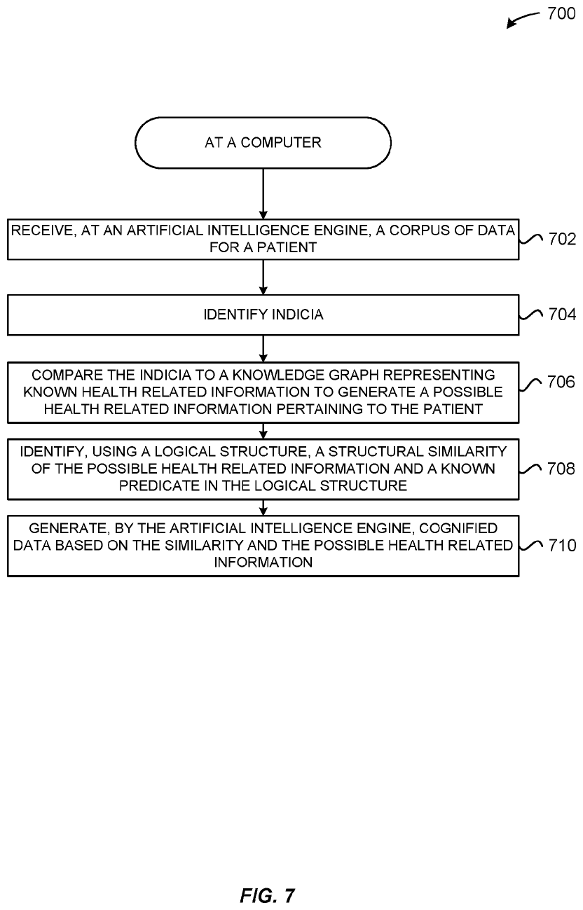

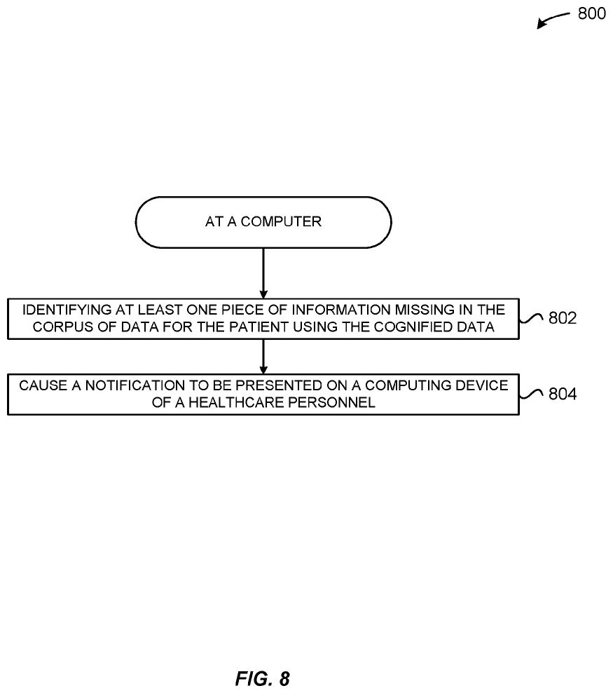

FIG. 7 shows a computer-implemented method 700 for generated cognified data using unstructured data. In some embodiments, the method 700 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 600 shown in FIG. 6. The method 700 may include operations that are implemented in computer instructions stored in a memory and executed by a processor of a computing device.

At block 702, the processing device may receive, at an artificial intelligence engine, a corpus of data for a patient. The corpus of data may represent unstructured data. The corpus of data may include a set of strings of characters. The corpus of data may be patient notes in an electronic medical record entered by a physician. In some embodiments, an application programming interface (API) may be used to interface with an electronic medical record system used by the physician. The API may retrieve one or more EMRs of the patient and extract the patient notes. The artificial intelligence engine may include the one or more machine learning models trained to generate cognified data based on unstructured data.

At block 704, the processing device may identify indicia. The indicia may be identified by processing the strings of characters. The indicia may include a phrase, a predicate, a subject, an object (e.g., direct, indirect), a keyword, a cardinal, a number, a concept, an objective, a noun, a verb, or some combination thereof.

At block 706, the processing device may compare the indicia to a knowledge graph representing known health related information to generate a possible health related information pertaining to the patient. In some embodiments, the indicia may be compared to numerous knowledge graphs each representing a different medical conditions. As discussed herein, the knowledge graphs may include respective nodes that include different known health related information about the medical conditions, and a logical structure that includes predicates that correlate the information in the respective knowledge graphs. The knowledge graphs and the logical structures may be generated by the one or more trained machine learning models using the known health related information. The knowledge graph may represent knowledge of a disease and the knowledge graph may include a set of concepts pertaining to the disease obtained from the known health related information and also includes relationships between the set of concepts. The known health related information associated with the nodes may be facts, concepts, complications, risks, causal effects, etc. pertaining to the medical conditions (e.g., diseases) represented by the knowledge graphs. The processing device may codify evidence-based health related guidelines pertaining to the diseases to generate the logical structures. The generated possible health related information may be a tag that is associated with the indicia in the unstructured data.

At block 708, the processing device may identify, using the logical structure, a structural similarity of the possible health related information and a known predicate in the logical structure. The structural similarity may be used to identify a certain pattern. The pattern may pertain to treatment, quality of care, risk adjustment, orders, referral, education and content patterns, and the like. The structural similarity and/or the pattern may be used to cognify the corpus of data.

At block 710, the processing device may generate, by the artificial intelligence engine, cognified data based on the structural similarity. In some embodiments, the cognified data may include a health related summary of the possible health related information. The health related summary may include conclusions, concepts, recommendations, identified gaps in the treatment plan, identified gaps in risk analysis, identified gaps in quality of care, and so forth pertaining to one or more medical conditions represented by one or more knowledge graphs that include the logic structure having the known predicate that is structurally similar to the possible health related information.

In some embodiments, generating the cognified data may include generating at least one new string of characters representing a statement pertaining to the possible health related information. Also, the artificial intelligence engine executed by the processing device may include the at least one new string of characters in the health related summary of the possible health related information. The statement may include a concept, conclusion, and/or recommendation pertaining to the possible health related information. The statement may describe an effect that results from the possible health related information.

FIG. 8 shows a method 800 for identifying missing information in a corpus of data, in accordance with various embodiments. In some embodiments, the method 800 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 600 shown in FIG. 6. The method 800 may include operations that are implemented in computer instructions stored in a memory and executed by a processor of a computing device.

At block 802, the processing device executing the artificial intelligence engine may identify at least one piece of information missing in the corpus of data for the patient using the cognified data. The at least one piece of information pertains to a treatment gap, a risk, gap, a quality of care gap, or some combination thereof.

At block 804, the processing device may cause a notification to be presented on a computing device of a healthcare personnel (e.g., physician). The notification may instruct entry of the at least one piece of information into the corpus of data (e.g., patient notes in the EMR). For example, if certain symptoms are described for a patient in the corpus of data and those symptoms are known to result from a certain medication currently prescribed to the patient, but the corpus of data does not indicate switching medications, then the at least one piece of information may identify a treatment gap and recommend switching medications to one that does not cause those symptoms.

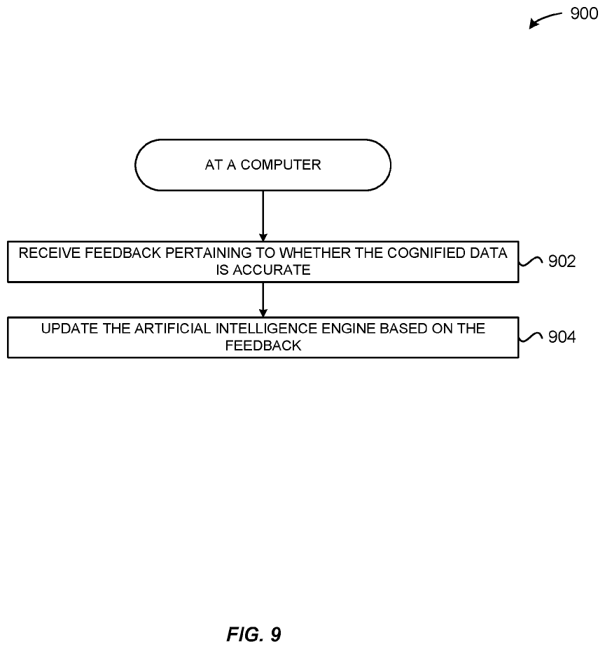

FIG. 9 shows a method 900 for using feedback pertaining to the accuracy of cognified data to update an artificial intelligence engine, in accordance with various embodiments. In some embodiments, the method 900 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 600 shown in FIG. 6. The method 900 may include operations that are implemented in computer instructions stored in a memory and executed by a processor of a computing device.

At block 902, the processing device may receive feedback pertaining to whether the cognified data is accurate. For example, the physician may be presented with the cognified data on a computing device, and the physician may review the cognified data. The physician may be presented with options to verify the accuracy of portions or all of the cognified data for the particular patient. For example, the physician may select a first graphical element (e.g., button, checkbox, etc.) next to portions of the cognified data that are accurate and may select a second graphical element next to portions of the cognified data that are inaccurate. If the second graphical element is selected, an input box may appear and a notification may be presented to provide a reason why the portion is inaccurate and to provide corrected information. The feedback may be transmitted to the cognitive intelligence platform.

At block 904, the processing device may update the artificial intelligence engine based on the feedback. A closed-loop feedback system may be implemented using these techniques. The feedback may enhance the accuracy of the cognified data as the artificial intelligence engine continues to learn and improve.

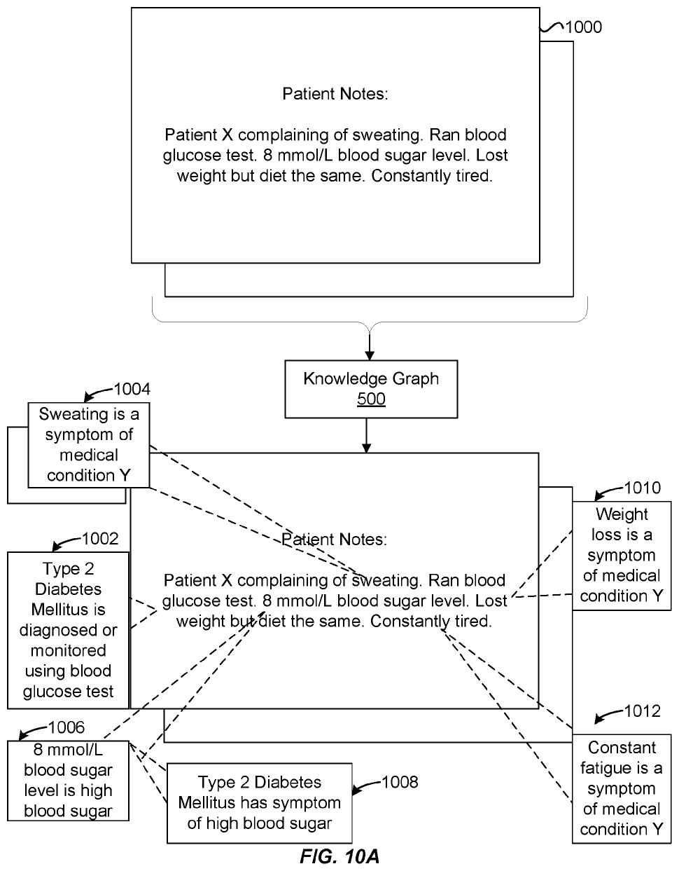

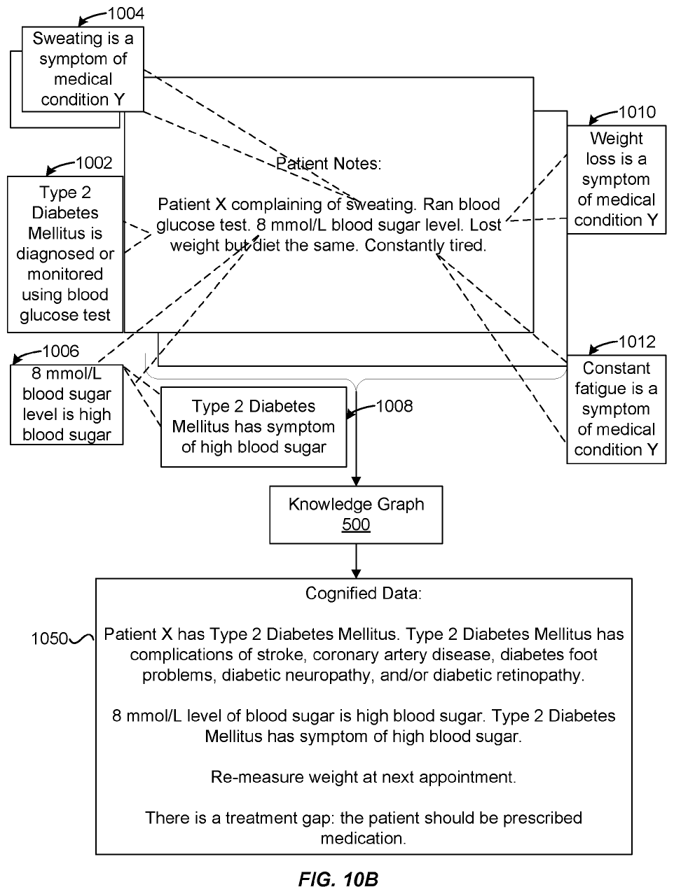

FIG. 10A shows a block diagram for using the knowledge graph 500 to generate possible health related information, in accordance with various embodiments. As depicted, a physician may have entered patient notes 2400 in one or more electronic medical records (EMRs). The EMRs may be provided directly to the cognitive intelligence engine 102 and/or retrieved using an application programming interface (API) from an EMR system used by the physician. The patient notes may be extracted from the EMRs. In some embodiments, numerous patient notes from numerous consultations may be processed, synthesized, and cognified using the disclosed techniques. In some embodiments, patient notes from a single consultation may be processed, synthesized, and cognified using the disclosed techniques. The patient notes may include a set of strings of characters that arranged in sentences, phrases, and/or paragraphs. The cognitive intelligence platform 102 may process the set of strings of characters to identify indicia comprising a phrase, a predicate, a keyword, a subject, an object, a cardinal, a number, a concept, or some combination thereof.

The cognitive intelligence platform 102, and in particular the artificial intelligence engine 109, may compare the indicia to numerous knowledge graphs 500 each representing a respective medical condition, such as diabetes, cancer, coronary artery disease, arthritis, just to name a few examples. The artificial intelligence engine 109 may be trained to generate possible health related information by constructing logical structures based on matched indicia and known health related information (health artifacts that are established based on information from a trusted source) represented in the knowledge graphs 500. The logical structures may be tagged to the indicia, as depicted in FIG. 10A.

The artificial intelligence engine 109 may identify the following example indicia: “Patient X”, “sweating”, “blood glucose test”, “8 mmol/L blood sugar level”, “lost weight”, “diet the same”, “constantly tired”. The artificial intelligence engine 109 may match the indicia with known health related information in the knowledge graph 500. For example, in the knowledge graph 500 depicted in FIG. 5, “blood glucose test”, is a known health related artifact that is used to test for Type 2 Diabetes Mellitus. Thus, various logical structures may be constructed by the artificial intelligence engine 109 that states “blood glucose test is used to test Type 2 Diabetes Mellitus”, “Type 2 Diabetes Mellitus is diagnosed or monitored using blood glucose test” (tag 2402), “blood glucose test measures blood sugar level”, and so forth.

The artificial intelligence engine 109 may generate other possible health related information for each of the indicia that matches known health related information in the knowledge graphs. For example, the artificial intelligence engine 109 generated example logical structure “Sweating is a symptom of medical condition Y” (tag 2404) for the indicia “sweating”. The artificial intelligence engine 109 may generate other possible health related information for “sweating”, such as “sweating is caused by running”, “sweating is a symptom of fever”. Further, the artificial intelligence engine 109 may elaborate on the generated possible health related information by generating further possible health related information. Based on generating “sweating is a symptom of medical condition Y” (where Y is the name of the medical condition), the artificial intelligence engine 109 may generate another logical structure “medical condition Y causes Z” (where Z is a health artifact such as another medical condition).

It should be understood that, although not shown, a logical structure may be included in the knowledge graph 500 that indicates “Type 2 Diabetes has normal blood sugar level 5-7 mmol/L”. An example possible health related information generated by the artificial intelligence engine 109 for the indicia “8 mmol/L blood sugar level” is “8 mmol/L blood sugar level is high blood sugar” (tag 2406) based on comparing the indicia to the known health related information about acceptable blood sugar levels in the knowledge graph 500. The artificial intelligence engine 109 may generate an additional possible health information based on tag 2406, and the additional possible health information may state “Type 2 Diabetes Mellitus has symptom of high blood sugar” (tag 2408).

An example possible health related information generated by the artificial intelligence engine 109 for the indicia “lost weight” may be “Weight loss is a symptom of medical condition Y” (tag 1010) where medical condition Y is any medical condition that causes weight loss. For example, any knowledge graph that includes “weight loss”, “loss of weight”, or some variant thereof as a health artifact may be identified and one or more possible health related information may be generated indicating that weight loss is a symptom of the medical condition represented by that knowledge graph.

An example possible health related information generated by the artificial intelligence engine 109 for the indicia “constantly tired” may be “Constant fatigue is a symptom of medical condition Y” (tag 1012) where medical condition Y is any medical condition that causes constant fatigue. For example, any knowledge graph that includes “fatigue”, “constant fatigue”, or some variant thereof as a health artifact may be identified and one or more possible health related information may be generated indicating that constant fatigue is a symptom of the medical condition represented by that knowledge graph.

The knowledge graphs that include a threshold number of matches between the indicia and the known health related matches in the knowledge graphs may be selected for further processing. The threshold may be any suitable number of matches. For example, in the depicted example, the knowledge graph 500 representing Type 2 Diabetes Mellitus may be selected because 3 tags (1002, 1006, and 1008) relate to that medical condition represented in the knowledge graph 500.

FIG. 10B shows a block diagram for using a logical structure to identify structural similarities with known predicates to generate cognified data, in accordance with various embodiments. The identification of structural similarities may be performed in parallel with the comparison of the indicia with the known health related information. In some embodiments, the generated possible health related information may be compared with the known predicates in the logical structures of the knowledge graphs. In some embodiments, predicates detected in the unstructured data may also be compared with the known predicates in the logical structures of the knowledge graphs. The artificial intelligence engine 500 may identify structural similarities between the possible health related information and the known predicates in the logical structures of the knowledge graphs. The artificial intelligence engine 500 may identify structural similarities between the detected predicates in the unstructured data and the known predicates in the logical structures of the knowledge graphs. In some embodiments, identifying structural similarities may refer to comparing the structure of the logical structure of the possible health related information to a known logical structure (known logical structure may refer to a logical structure established based on a trusted source), such as determining whether the subjects are the same or substantially similar, the predicates are the same or substantially similar, the objects are the same or substantially similar, and so forth.

For example, the knowledge graph 500 includes the logical structure “Type 2 Diabetes Mellitus has symptom high blood sugar”. Comparing the possible health related information represented by tag 1008 “Type 2 Diabetes Mellitus has symptom of high blood sugar” to the known logical structure in the knowledge graph 500 results in identifying a structurally similarity between the two. Accordingly, the knowledge graph 500 may be selected for further processing.

In some embodiments, the structural similarities detected may be used to identify patterns. For example, a treatment pattern for diabetes may be detected if a blood glucose test is used, a patient is prescribed a certain medication, and the like. In some embodiments, gaps in the unstructured data may be identified based on the patterns detected. For example, if a person is determined to have a certain medical condition based on the treatment pattern identified, and it is known based on evidence-based guidelines that a certain medication should be prescribed for that treatment pattern, the artificial intelligence engine 109 may indicate there is a treatment gap if that medication has not been prescribed yet.

The knowledge graphs selected when comparing the indicia to the known health related information and the knowledge graphs selected when identifying structural similarities between the known logical structure and the possible health related information may be compared to determine whether there are overlaps. As discussed above, the knowledge graph 500 representing Type 2 Diabetes Mellitus overlaps as being selected during both operations. As a result, the knowledge graph 500 may be used for cognification. In some embodiments, any of the knowledge graphs selected during either operation may be used for cognification.

In some embodiments, the selected knowledge graphs may be used to generate cognified data 1050. Further, the possible health related information and the matching logical structures may be used to generate the cognified data 1050. The cognified data 1050 may include a health related summary of the possible health related information. In some embodiments, the cognified data 1050 may include conclusions, statements of facts, concepts, recommendations, identified gaps in the unstructured data that was processed, and the like.

In some embodiments, the cognified data 1050 may be used to generate a diagnosis of a medical condition for a patient. For example, if there are a threshold number of identified structural similarities between the known logical structures and the possible health related information and/or if there are a threshold number of matches between indicia and known health related information for a particular medical condition, a diagnosis may be generated for that particular medical condition. If there are numerous medical conditions identified after performing the cognification, the numerous medical conditions may be indicated as potential candidates for diagnosis. In the ongoing example, the knowledge graph 500 was selected as the overlapping knowledge graph and satisfies the threshold number of identified structural similarities and/or the threshold number of matches. Accordingly, a diagnosis that Patient X has Type 2 Diabetes Mellitus may be generated. The cognified data 1050 may include the diagnosis, as depicted.

When generating the cognified data, other health related information in the selected knowledge graph 500 that was not included in the unstructured data may be inserted. That is, sentences may be constructed using the known health related information and the predicates in the knowledge graph 50. For example, the unstructured data did not indicate any information pertaining to complications of Type 2 Diabetes Mellitus. However, as depicted in the knowledge graph 500 of FIG. 5, there is a logical structure that specifies “Type 2 Diabetes Mellitus has complications of stroke, coronary artery disease, diabetes foot problems, diabetic neuropathy, and/or diabetic retinopathy”. As depicted, this construction of the logical structure is included in the cognified data 1050 by the artificial intelligence engine 109.

The cognified data 1050 may also include the tag 2406 (“8 mmol/L level of blood sugar is high blood sugar. Type 2 Diabetes Mellitus has symptom of high blood sugar”) that was generated for the unstructured data based on the known health information in the knowledge graph 500. The artificial intelligence engine 109 may generate a recommendation based on the lost weight indicia indicated in the unstructured data. The recommendation may state “Re-measure weight at next appointment.” In addition, as discussed above, the artificial intelligence engine 109 may identify certain gaps. For example, the diagnosis that is generated indicates that the patient has Type 2 Diabetes Mellitus. The unstructured data does not indicate that medication is prescribed. However, the knowledge graph 500 specifies that Type 2 Diabetes Mellitus is treated by “Diabetes Medicines”. Accordingly, a treatment gap may be identified by the artificial intelligence engine 109 based on treatment patterns codified in the knowledge graph 500, and a statement may be constructed and inserted in the cognified data 1050. The statement may state “There is a treatment gap: the patient should be prescribed medication.”

The cognified data 1050 may be transmitted by the cognitive intelligence platform 102 to a computing device of the service provider 112, such as the physician who entered the unstructured data. As depicted, the cognified data 1050 may be instilled with intelligence, knowledge, and logic using the disclosed cognification techniques. The physician may quickly review the cognified data 1050 without having to review numerous patient notes from various EMRs. In some embodiments, the physician may be presented with options to verify portions or all of the cognified data 1050 is accurate. The feedback may be transmitted to the cognitive intelligence platform 102 and the artificial intelligence engine 109 may update its various machine learning models using the feedback.

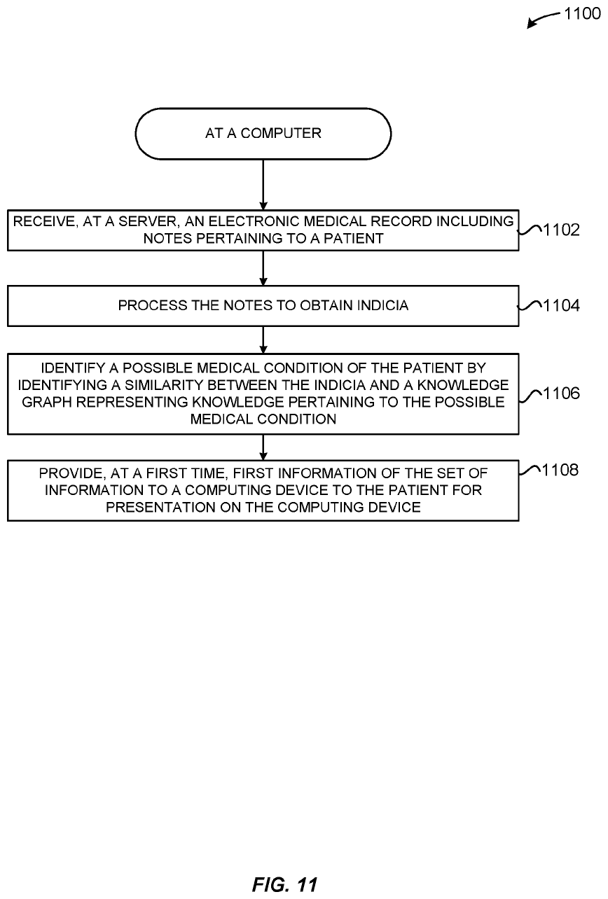

FIG. 11 shows a method 1100 for providing first information pertaining to a possible medical condition of a patient to a computing device, in accordance with various embodiments. In some embodiments, the method 1100 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 600 shown in FIG. 6. The method 1100 may include operations that are implemented in computer instructions stored in a memory and executed by a processor of a computing device.

At block 1102, the processing device of a server may receive an electronic medical record (EMR) including notes pertaining to a patient. The EMR may be transmitted directly to the server from a computing device of the physician that entered the notes, and/or the EMR may be obtained using an application programming interface (API) interfacing with an EMR system used by the physician that entered the notes. In some embodiments, the server may receive text input by the patient. For example, the text input by the user may include symptoms the patient is experiencing and ask a question pertaining to what medical condition the patient may have. The operations of method 1100 may be used to similarly provide information to the patient based on identifying the possible medical condition using the cognification techniques.

At block 1104, the processing device may process the notes to obtain indicia including a subject, an object, a word, a cardinal, a phrase, a concept, a sentence, a predicate, or some combination thereof. Textual analysis may be performed to extract the indicia. Processing the patient notes to obtain the indicia may further include inputting the notes into an artificial intelligence engine 109 trained to identify the indicia in text based on commonly used indicia pertaining to the possible medical condition. The artificial intelligence engine 109 may determine commonly used indicia for various medical conditions based on evidence-based guidelines, clinical trial results, physician research, or the like that are input to one or more machine learning models.

At block 1106, the processing device may identify a possible medical condition of the patient by identifying a similarity between the indicia and a knowledge graph representing knowledge pertaining to the possible medical condition. The knowledge graph may include a set of nodes representing the set of information pertaining to the possible medical condition. The set of nodes may also include relationships (e.g., predicates) between the set of information pertaining to the possible medication condition. In some embodiments, identifying the possible medical condition may include using a cognified data structure generated from the notes of the patient. The cognified data structure may include a conclusion based on a logic structure representing evidence-based guidelines pertaining to the possible medical condition.

In some embodiments, the similarity may pertain to a match between the indicia and a health artifact (known health related information) included in the knowledge graph 500. For example, “high blood pressure” may be extracted as indicia from the sentence “Patient X has high blood pressure”, and “high blood pressure” is a health artifact at a node in the knowledge graph 500 representing Type 2 Diabetes Mellitus.

In some embodiments, the similarity may pertain to a structural similarity between the logical structure (e.g., “Type 2 Diabetes has symptoms of High Blood Pressure) and the indicia (e.g., “Patient X has symptoms of High Blood Pressure”) that is included in the unstructured data. If the subject, predicates, and/or objects of the logical structure and the indicia match or substantially match (e.g., “has symptoms of High Blood Pressure” match between the logical structure and the indicia, also “Type 2 Diabetes has symptoms of High Blood Pressure” and “Patient X has symptoms of High Blood Pressure” substantially match), then the knowledge graph 500 including the logical structure is a candidate for a possible medical condition. In some embodiments, a combination of similarities identified between the match between the indicia and the health artifact and between the logical structure and the indicia may be used to identify a possible medical condition and/or cognify the unstructured data.

An artificial intelligence engine 109 may be used to identify the possible medical condition by identifying the similarity between the indicia and the knowledge graph. The artificial intelligence engine 109 may be trained using feedback from medical personnel. The feedback may pertain to whether output regarding the possible medical conditions from the artificial intelligence engine 109 are accurate for input including notes of patients.

At block 1108, the processing device may provide, at a first time, first information of the set of information to a computing device of the patient for presentation of the computing device, the first information being associated with a root node of the set of nodes. In some embodiments, the first information may pertain to a name of the possible medical condition. As depicted in the knowledge graph 500 of FIG. 5, the root node is associated with the name of the medical condition “Type 2 Diabetes Mellitus”. In some embodiments, the first information may pertain to a definition of the possible medical condition, instead of or in addition to the name of the possible medical condition.

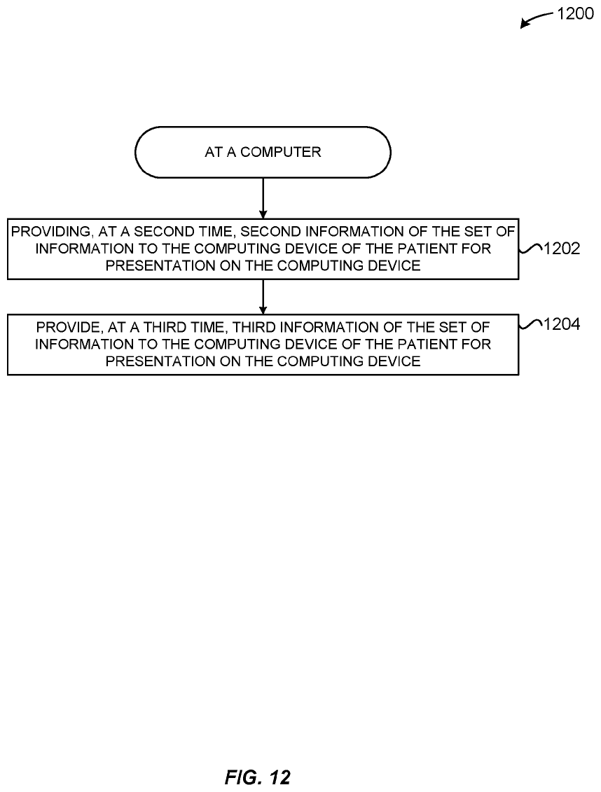

FIG. 12 shows a method 1200 for providing second and third information pertaining to a possible medical condition of a patient to a computing device, in accordance with various embodiments. In some embodiments, the method 1200 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 600 shown in FIG. 6. The method 1200 may include operations that are implemented in computer instructions stored in a memory and executed by a processor of a computing device.

At block 1202, the processing device may provide, at a second time, second information of the set of information to the computing device of the patient for presentation on the computing device. The second information may be associated with a second node of the set of nodes, and the second time may be after the first time. The second information may be different than the first information. The second information may pertain to how the possible medical condition affects people, signs and symptoms of the possible medical condition, a way to treat the possible medical condition, a progression of the possible medical condition, complications of the possible medical condition, or some combination thereof. The second time may be selected based on when the second information is relevant to a stage of the possible medical condition. The second time may be preconfigured based on an amount of time elapsed since the first time.

At block 1204, the processing device may provide, at a third time, third information of the set of information to the computing device of the patient for presentation on the computing device of the patient. The third information may be associated with a third node of the set of nodes, and the third time may be after the second time. The third information may be different than the first information and the second information. The third information may pertain to how the possible medical condition affects people, signs and symptoms of the possible medical condition, a way to treat the possible medical condition, a progression of the possible medical condition, complications of the possible medical condition, or some combination thereof. The third time may be selected based on when the third information is relevant to a stage of the possible medical condition. The third time may be preconfigured based on an amount of time elapsed since the second time.

This process may continue until each node of the knowledge graph 500 are traversed to provide relevant information to the patient at relevant times until all information associated with the set of nodes has been delivered to the computing device of the patient. In this way, the patient may not be overwhelmed with a massive amount of information at once. Further, memory resources of the computing device of the patient may be saved by regulating the amount of information that is provided.

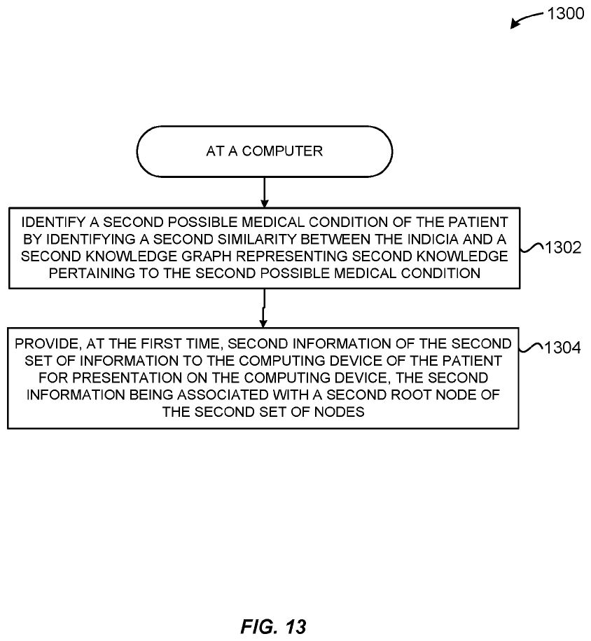

FIG. 13 shows a method 1300 for providing second information pertaining to a second possible medical condition of the patient, in accordance with various embodiments. In some embodiments, the method 1300 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 600 shown in FIG. 6. The method 1300 may include operations that are implemented in computer instructions stored in a memory and executed by a processor of a computing device.

At block 1302, the processing device may identify a second possible medical condition of the patient by identifying a second similarity between the indicia and a second knowledge graph representing second knowledge pertaining to the second possible medical condition. In some embodiments, the second similarity may pertain to a match between the indicia and a health artifact (known health related information) included in the second knowledge graph. For example, “vomiting” may be extracted as indicia from the sentence “patient has symptom of vomiting”, and “vomiting” is a health artifact at a node in the second knowledge graph representing the flu. In some embodiments, the second similarity may pertain to a second structural similarity between a second logical structure (e.g., “Flu has symptom of vomiting) and the possible health information (e.g., “has symptom of vomiting”) that is included in the unstructured data. In some embodiments a combination of the similarities between the indicia and the health artifact and between the logical structure and the possible health information may be used to identify the second possible medical condition and/or cognify the unstructured data.

At block 1304, the processing device may provide, at the first time, second information of the second set of information to the computing device of the patient for presentation on the computing device, the second information being associated with a second root node of the second set of nodes. The second information may be provided with the first information at the first time. In some embodiments, a user interface on the computing device of the patient may present the first information and the second information concurrently on the same screen. For example, the user interface may present that the possible medical conditions include “Type 2 Diabetes Mellitus” and the “flu”. It should be understood that any suitable number of possible medical conditions may be identified using the cognification techniques and the information related to those medical conditions may be provided to the computing device of the patient on a regulated basis.

In some embodiments, the patient may be presented with options to indicate whether the information provided at the various times was helpful. The feedback may be provided to the artificial intelligence engine 109 to update one or more machine learning models to improve the information that is provided to the patients.

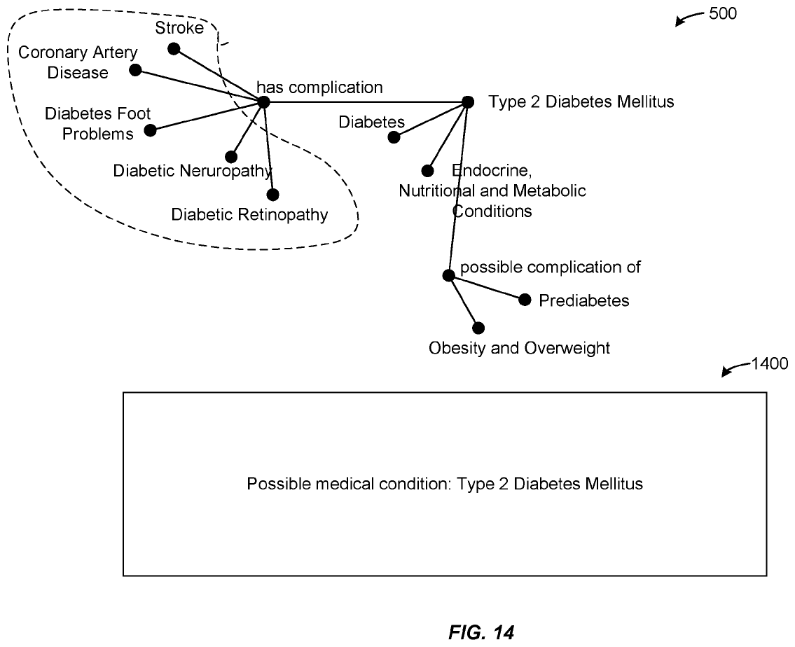

FIG. 14 shows an example of providing first information of a knowledge graph 500 representing a possible medical condition, in accordance with various embodiments. In the depicted example, just a portion of the knowledge graph 500 representing Type 2 Diabetes Mellitus is depicted. Based on the patient notes entered by the physician and/or the text input by the patient, the artificial intelligence engine 109 may extract indicia. Using the indicia, the artificial intelligence engine 109 may identify a possible medical condition of the patient by identifying at least one similarity between the indicia and the knowledge graph 500. It should be understood that the artificial intelligence engine 109 identified Type 2 Diabetes Mellitus as the possible medical condition based on the similarity between the indicia and the knowledge graph 500 using the cognification techniques described herein.

Accordingly, at a first time, the cognitive intelligence platform 102 may provide first information associated with the root node of the knowledge graph 500. The root node may be associated with the name “Type 2 Diabetes Mellitus” of the medical condition. A user interface of the computing device of the patient may present the first information “Possible medical condition: Type 2 Diabetes Mellitus” at the first time.

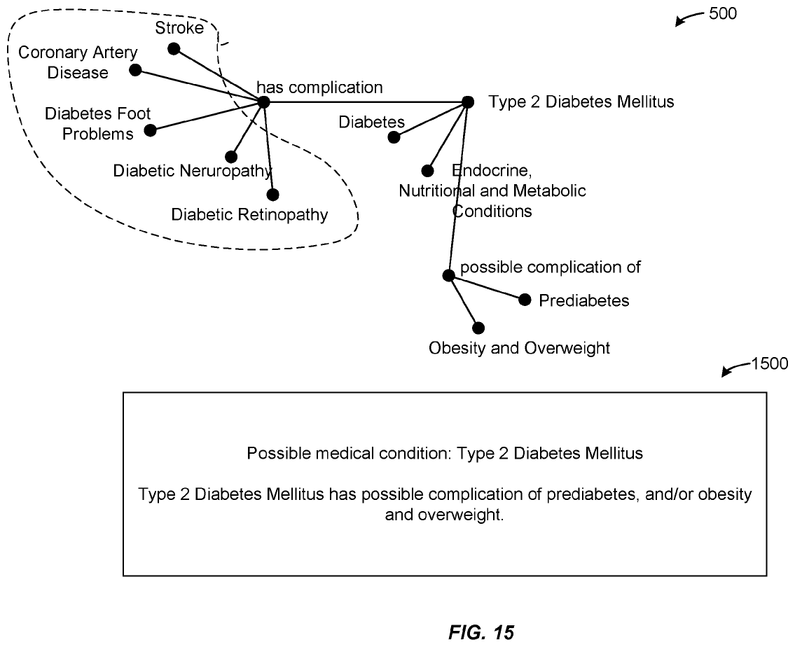

FIG. 15 shows an example of providing second information of the knowledge graph 500 representing the possible medical condition, in accordance with various embodiments. The second information may be provided at a second time subsequent to the first time the first information was provided. The second information may be associated with at least a second node representing a health artifact of the knowledge graph 500. The second information may be different than the first information. The second information may combine a predicate of a node that connects the second node representing the health artifact to the root node. For example, the second information may include “Type 2 Diabetes Mellitus has possible complication of prediabetes, or obesity and overweight.” The second information may be presented on the user interface 1500 with the first information, as depicted. In some embodiments, just the second information may be presented on the user interface 1500 and the first information may be deleted from the user interface 1500.

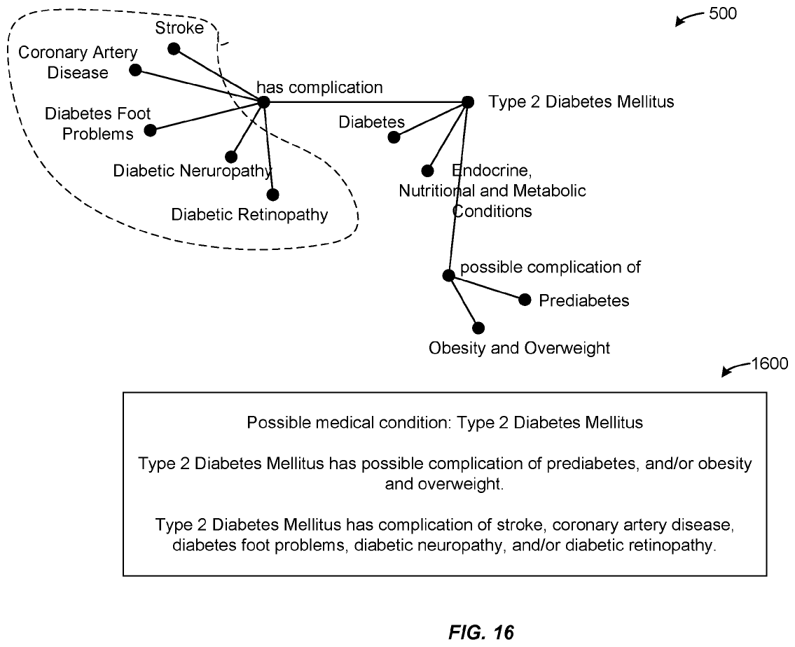

FIG. 16 shows an example of providing third information of the knowledge graph representing the possible medical condition, in accordance with various embodiments. The third information may be provided at a third time subsequent to the second time the second information was provided. The third information may be associated with at least a third node representing a health artifact of the knowledge graph 500. The third information may be different than the first information and the second information. The third information may combine a predicate of a node that connects the third node representing the health artifact to the root node. For example, the third information may include “Type 2 Diabetes Mellitus has complication of stroke, coronary artery disease, diabetes foot problems, diabetic neuropathy, and/or diabetic retinopathy.” The third information may be presented on the user interface 1600 with the first information and/or the second information, as depicted. In some embodiments, just the third information may be presented on the user interface 1600, and the first information and the second information may be deleted from the user interface 1600. In some embodiments, any combination of the first, second, and third information may be presented on the user interface 1600.

In some embodiments, the various health artifacts represented by each node in the knowledge graph 500 may be provided to the computing device of the patient until all of the information in the knowledge graph 500 is provided. Additionally, if the knowledge graph 500 contains a link to another knowledge graph representing a related medical condition, the information included in that other knowledge graph may be provided to the patient. At any time, the patient may request to stop receiving information about the possible medical condition and no additional information will be provided. If the patient desires additional information faster, the patient may be presented with an option to obtain the next set of information at any time.

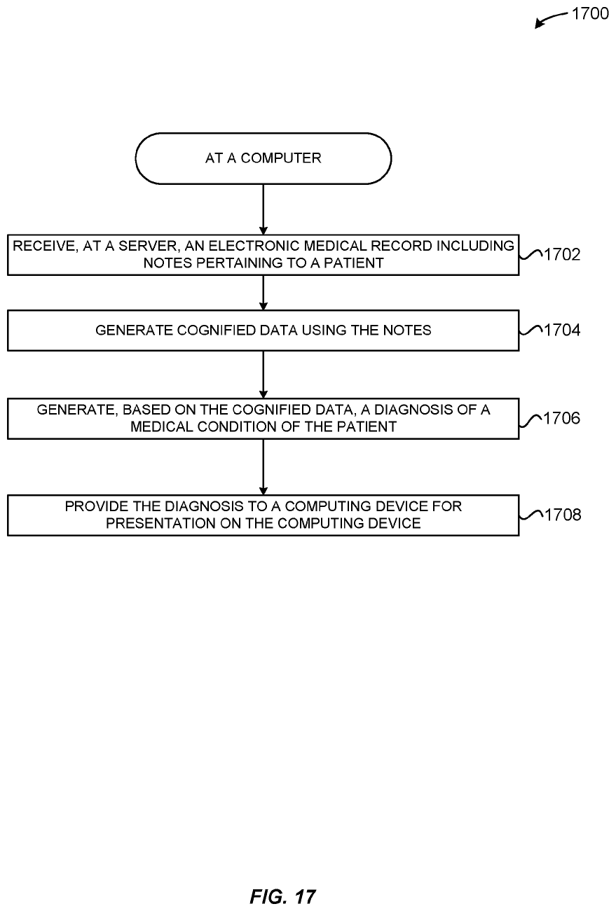

FIG. 17 shows a method 1700 for using cognified data to diagnose a patient, in accordance with various embodiments. In some embodiments, the method 1700 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 600 shown in FIG. 6. The method 1700 may include operations that are implemented in computer instructions stored in a memory and executed by a processor of a computing device.

At block 1702, the processing device of a server may receive an electronic medical record including notes pertaining to a patient. The notes may include strings of characters arranged in sentences and/or paragraphs. The processing device may process the strings of characters and identify, in the notes, indicia including a phrase, a predicate, a subject, an object, a cardinal, a number, a concept, or some combination thereof. In some embodiments, the notes may be processed to obtain the indicia by inputting the notes into the artificial intelligence engine 109 trained to identify the indicia in text based on commonly used indicia pertaining to the medical condition.

At block 1704, the processing device may generate cognified data using the notes. The cognified data may include a health summary of a medical condition. Generating the cognified data may further include detecting the medical condition by identifying a similarity between the indicia and a knowledge graph. For example, in some embodiments, the similarity may pertain to a match between the indicia and a health artifact (known health related information) included in the knowledge graph 500. For example, “high blood pressure” may be extracted as indicia from the sentence “Patient X has high blood pressure”, and “high blood pressure” is a health artifact at a node in the knowledge graph 500 representing Type 2 Diabetes Mellitus. In some embodiments, the similarity may pertain to a structural similarity between the logical structure (e.g., “Type 2 Diabetes has symptoms of High Blood Pressure) and possible health related information generated using the identified indicia or subjects, predicates, and/or objects (e.g., “Patient X has symptoms of High Blood Pressure”) that is included in the unstructured data. In some embodiments, a combination of similarities between the indicia and the health artifact, and between the logical structure and the indicia/possible health related information may be used to detect the medical condition.

At block 1706, the processing device may generate, based on the cognified data, a diagnosis of the medical condition of the patient. The diagnosis may at least identify a type of the medical condition that is detected using the cognified data. The diagnosis may be generated if a threshold number of matches between the indicia and health artifacts in the knowledge graph are identified, and/or if a threshold number of structural similarities are identified between logical structures of the knowledge graph and indicia/possible health information generated for the unstructured data. For example, the threshold numbers may be configurable and set based on a confidence level that the health artifacts that match the indicia and/or the logical structures that are similar to the indicia/possible health related information are correlated with the particular medical condition. The threshold numbers may be based on information from trusted sources, such as physicians having medical licenses.

In some embodiments, the processing device may use an artificial intelligence engine 109 that is trained using feedback from medical personnel. The feedback may pertain to whether output regarding diagnoses from the artificial intelligence engine 109 are accurate for input including notes of patients. The cognified data may include a conclusion that is identified based on a logical structure in the knowledge graph 500, where the logical structure represents codified evidence-based guidelines pertaining to the medical condition.

At block 1708, the processing device may provide the diagnosis to a computing device of a patient and/or a physician for presentation on the computing device. The diagnosis may be included in the cognified data. The physician may review the diagnosis and may provide feedback via graphical element(s) whether the diagnosis is accurate. The feedback may be received by the artificial intelligence engine 109 and used to update the one or more machine learning models used by the artificial intelligence engine 109 to cognify data and generate diagnoses.

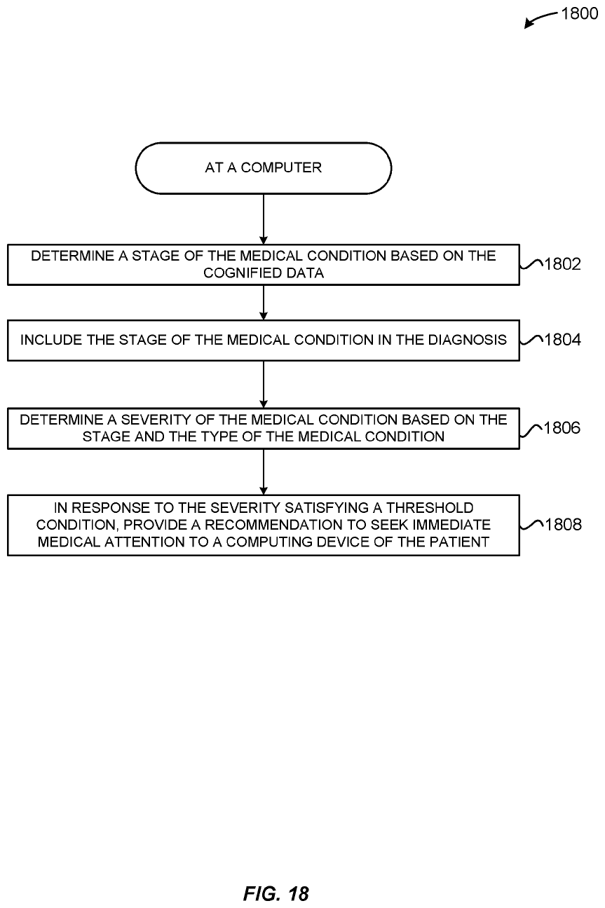

FIG. 18 shows a method 1800 for determining a severity of a medical condition based on a stage and a type of the medical condition, in accordance with various embodiments. In some embodiments, the method 1800 is implemented on a cognitive intelligence platform. In some embodiments, the cognitive intelligence platform is the cognitive intelligence platform 102 as shown in FIG. 1. In some embodiments, the cognitive intelligence platform is implemented on the computing device 600 shown in FIG. 6. The method 1800 may include operations that are implemented in computer instructions stored in a memory and executed by a processor of a computing device.

At block 1802, the processing device may determine a stage of the medical condition diagnosed based on the cognified data. The stage of the medical condition may be determined based on information included in the cognified data. For example, the information in the cognified data may be indicative of the particular stage of the medical condition. Such stages may include numerical values (e.g., 1, 2, 3, 4, etc.), descriptive terms (e.g., chronic, acute, etc.), or any suitable representation capable of indicating different progressions in a range (e.g., from low to high, or from mild to severe, etc.).

The artificial intelligence engine 109 may be trained to identify the stage based on the information in the cognified data. For example, if certain symptoms are present, certain blood levels are present, certain vital signs are present, or the like for a particular medical condition, the artificial intelligence engine 109 may determine that the medical condition has reached a certain stage. The artificial intelligence engine 109 may be trained on evidence-based guidelines that correlate the various information with the particular stages. For example, it may be known that a particular stage of cancer involves symptoms such as weight loss, lack of appetite, bone pain, dry cough or shortness of breath, or some combination thereof. If those symptoms are identified for the medical condition diagnosed (cancer) for the patient, then that particular stage may be determined.

At block 1804, the processing device may include the stage of the medical condition in the diagnosis. For example, the processing device may indicate the diagnosis is the “Patient X has stage 4 breast cancer”. At block 3206, the processing device may determine a severity of the medical condition based on the stage and the type of the medical condition. If the stage is relatively low and the medical condition is easily treatable, then the severity may be low. If the stage is relatively high (chronic) and the medical condition is difficult to treat (cancer), then the severity may be high.

At block 1808, in response to the severity satisfying a threshold condition, the processing device may provide a recommendation to seek immediate medical attention to a computing device of the patient. The threshold condition may be configurable. In some embodiments, the threshold condition may be set based on information from a trusted source (e.g., evidence-based guidelines, clinical trial results, physician research, and the like).

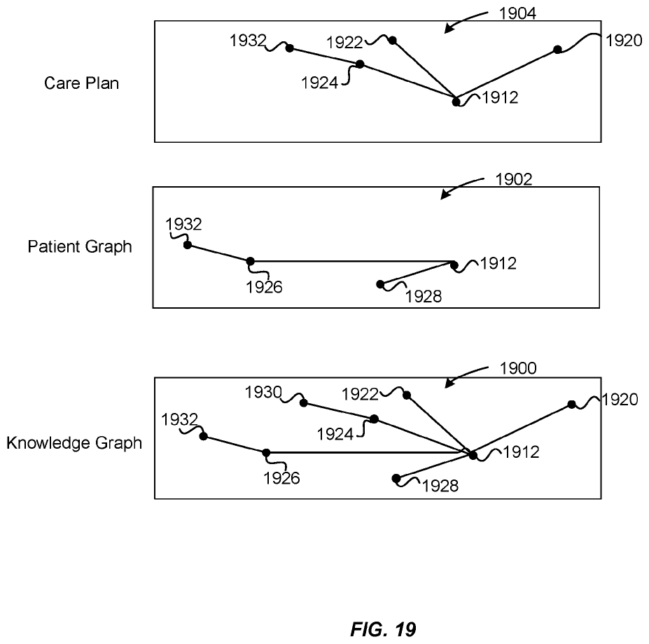

FIG. 19 shows an example of a knowledge graph 1900, a patient graph 1902, and a care plan 1904, in accordance with various embodiments. The knowledge graph 1900 may pertain to any suitable medical condition and include numerous elements (e.g., health artifacts) represented by nodes and relationships between the nodes represented by edges. For example, the knowledge graph 1900 includes a root node 1912; a first layer of nodes 1920, 1922, 1924, 1926, and 1928; and a second layer of nodes 1930, and 1932. The root node 1912 may include information pertaining to a type of the medical condition, such as “Multiple Sclerosis”. The edges connecting the root node 1912 to the first layer of nodes 1920, 1922, 1924, 1926, and 1928 may represent a relationship between the root node 1912 and the first layer of nodes 1920, 1922, 1924, 1926, and 1928. For example, the edge connecting the root node 1912 and 1920 may represent a relationship “has symptoms of” and the node 1920 may represent a health artifact “tingling and numbness”. The knowledge graph 1900 may include a superset of curated medical knowledge of the medical condition represented by the nodes and relationships pertaining to the medical condition.

The patient graph 1902 may be tailored for a particular user and may correspond to the condition represented by the knowledge graph 1900. For example, the patient graph 1902 may correspond to the medical condition “Multiple Sclerosis”. In some embodiments, the nodes in the patient graph 1902 may represent the health artifacts (e.g., actions, interactions, content, concepts, facts, protocols, evidence-based guidelines, etc.) which the user has performed, interacted, experienced, reported, consumed, been treated for, been diagnosed, and/or been prescribed. For example, the node 1928 may represent a particular test for Multiple Sclerosis. The user may have performed the particular test for Multiple Sclerosis. As such, the node 1928 is included in the patient graph 1902. The node 1928 may include a type of the particular test, a timestamp of the particular test, a result of the particular test, and the like.

Nodes 1926 and 1932 may correspond to other health artifacts which the user has performed, interacted, consumed, been treated for, been diagnosed, and/or been prescribed. As such, the nodes 1926 and 1932 are included in the patient graph 1902.

In the depicted example, the user may not have interacted with and/or performed the health artifacts associated with the nodes 1920, 1922, 1924, and 5630 in the knowledge graph for Multiple Sclerosis. Accordingly, the nodes 1920, 1922, 1924, and 5630 are not included in the patient graph 1902 for Multiple Sclerosis for the user. For example, the user may not have performed the action of performing a disease-modify therapy technique for treating Multiple Sclerosis. The health artifact for the disease-modifying therapy technique may be represented by node 1922, and thus, node 1922 is not included in the patient graph 1902.

The cognitive intelligence platform 102 may compare the patient graph 1902 to the knowledge graph 1900 to determine which areas of the condition Multiple Sclerosis to manage to generate the care plan 1904. Further, the cognitive intelligence platform 102 may consider the areas the user selected to manage when generating the care plan 1904. The patient graph 1902 may be projected onto the knowledge graph 1900. Overlapping nodes that are included in both the patient graph 1902 and the knowledge graph 1900 may be identified (e.g., highlighted in a first color). Further, nodes that are included in the knowledge graph 1900 and not included in the patient graph 1902 may also be identified (e.g., highlighted in a second color).

In some embodiments, the nodes that are present in the knowledge graph 1900 and not present in the patient graph 1902 may be selected to include in the care plan 1904. As depicted, nodes 1920, 1922, 1924, and 1932 are present in the knowledge graph 1900 and not in the patient graph 1902. Accordingly, the care plan 1904 may be generated to include the root node 1912 and the nodes 1920, 1922, 1924, and 1932. One or more action instructions may be generated and associated with each of the nodes 1920, 1922, 1924, and 1932.

For example, node 1920 may represent medications to take for the condition, and an action instruction may be generated to recommend the user discuss being prescribed a different medication for the condition. Other action instructions pertaining to various health artifacts may include scheduling a follow-up appointment, performing a certain test for the condition, reading certain recommended curated medical content pertaining to the condition, performing certain self-care treatments, and the like. In some embodiments, nodes may be selected to include in the care plan 1904 based on the areas of the condition the user selected to manage as well as the number of the areas of the condition the user selected to manage.

The care plan 1904 may be converted into natural language for each particular role. For example, the natural language representing the care plan 1904 may be tailored for providing action instructions to a user, the natural language representing the care plan 1904 may be tailored for providing action instructions to a medical personnel, and the natural language representing the care plan 1904 may be tailored for providing action instructions to an administrator. For example, the natural language conversion of the care plan 1904 may include an action instruction for the patient that specifies “Discuss changing medications with your physician”. In another example, the natural language conversion of the care plan 1904 may include an action instruction for the medical personnel that specifies “Discuss changing medications with the patient”. Each respective natural language conversion representing the care plan 1904 may be presented on the respective patient viewer, clinic viewer, and administrator viewer. The natural language conversion may be in text format and presented on the various viewers and/or may be in audio format and may be output by a speaker of a computing device.

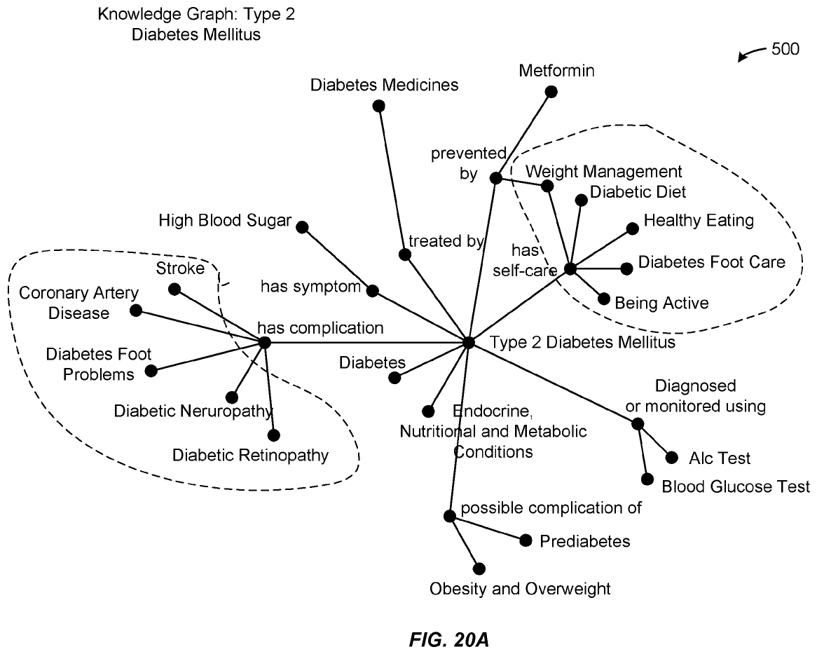

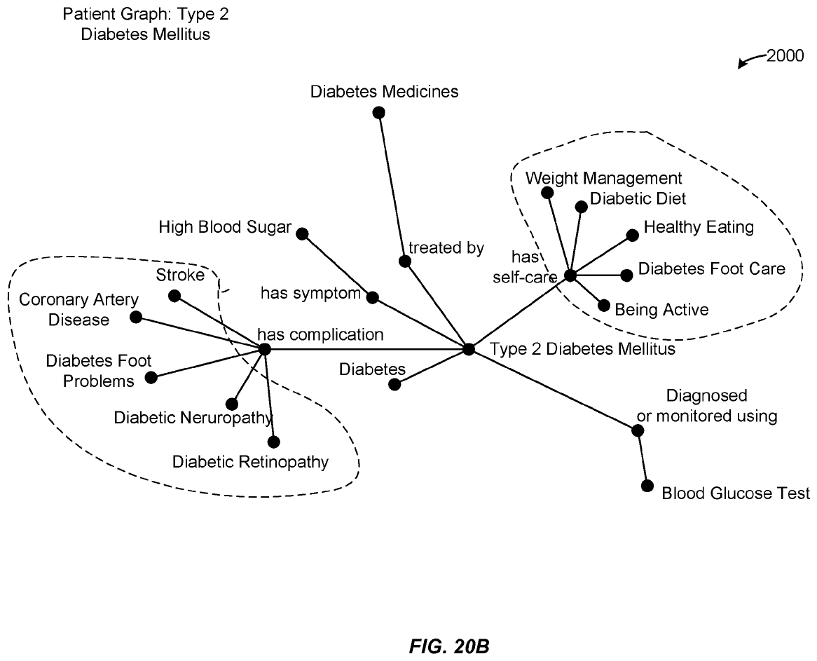

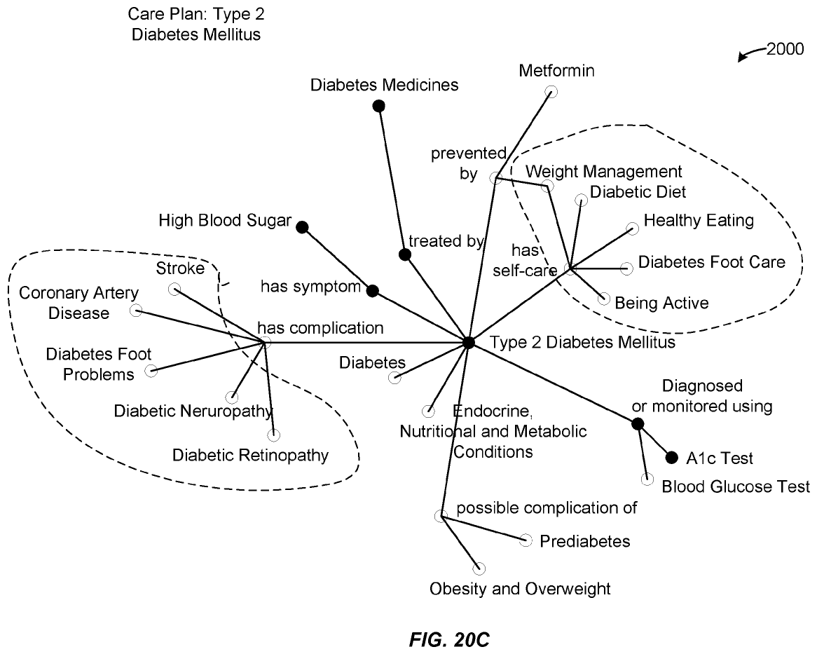

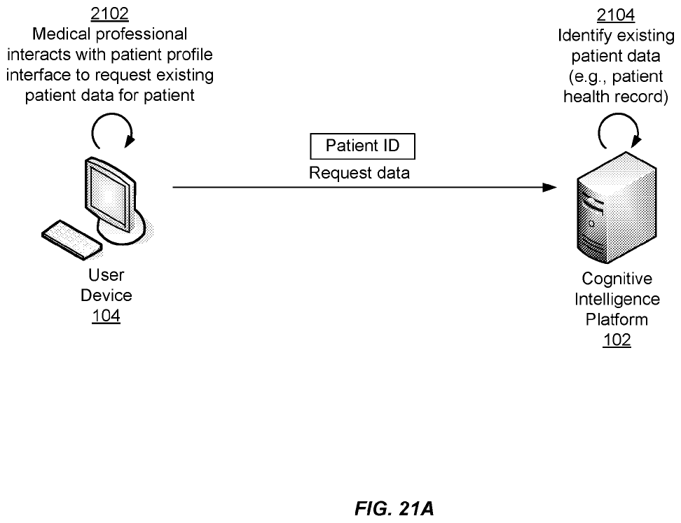

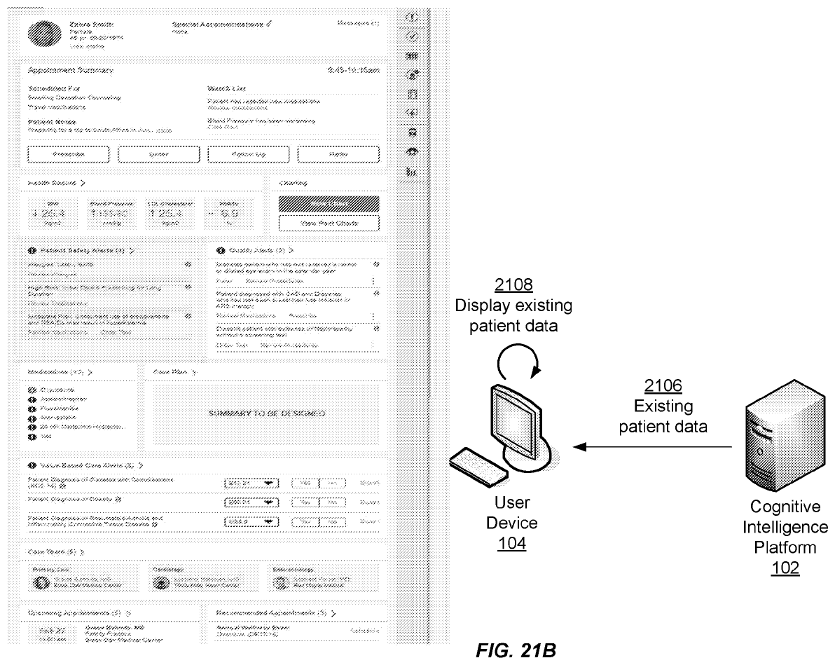

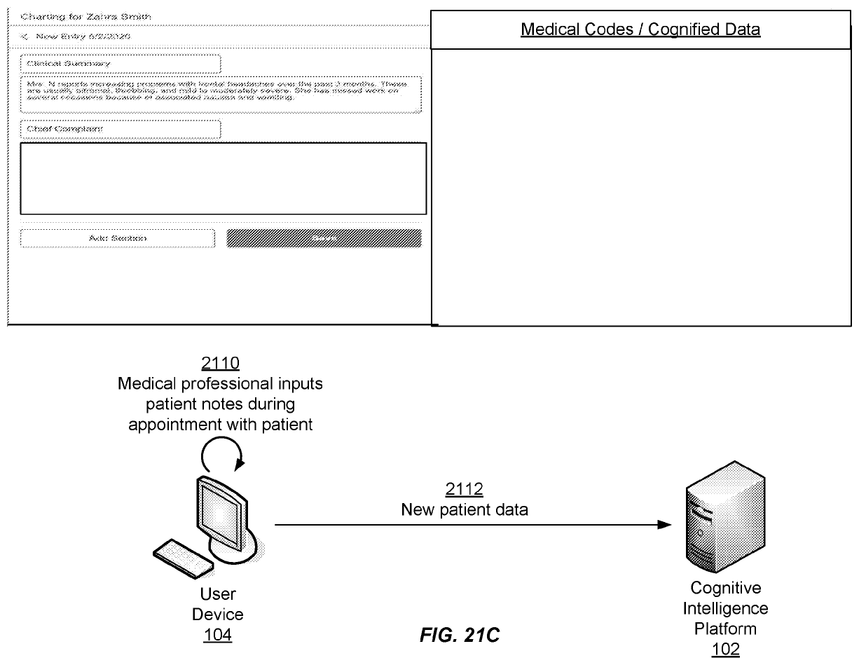

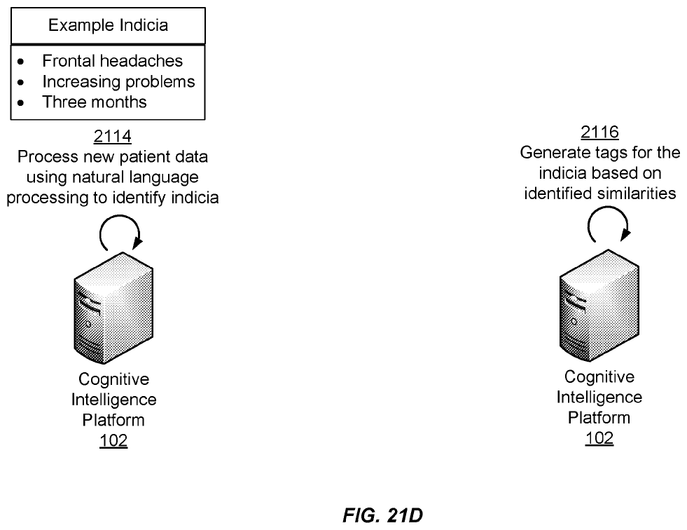

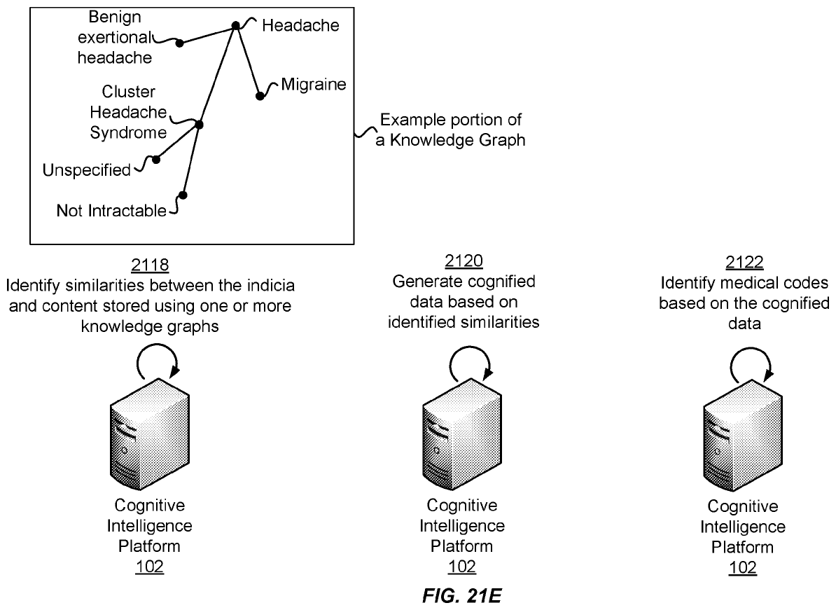

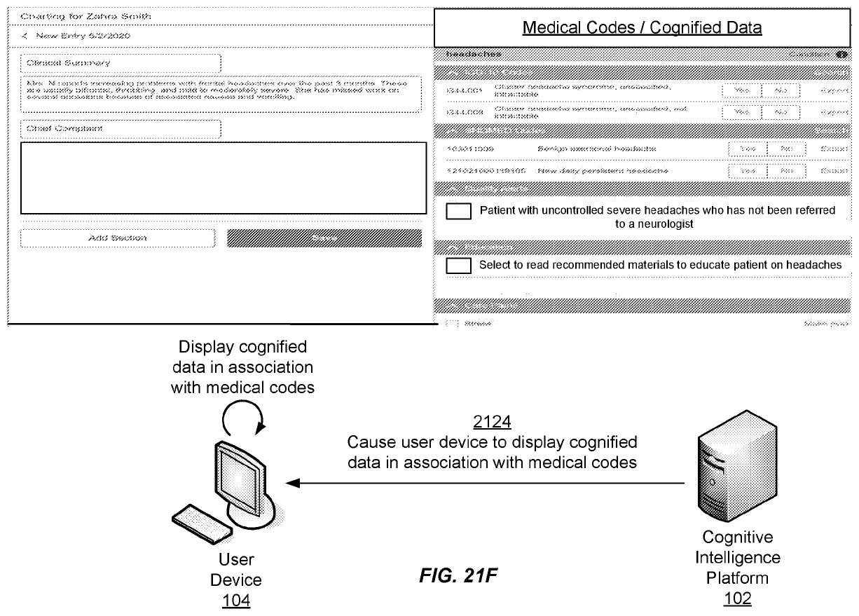

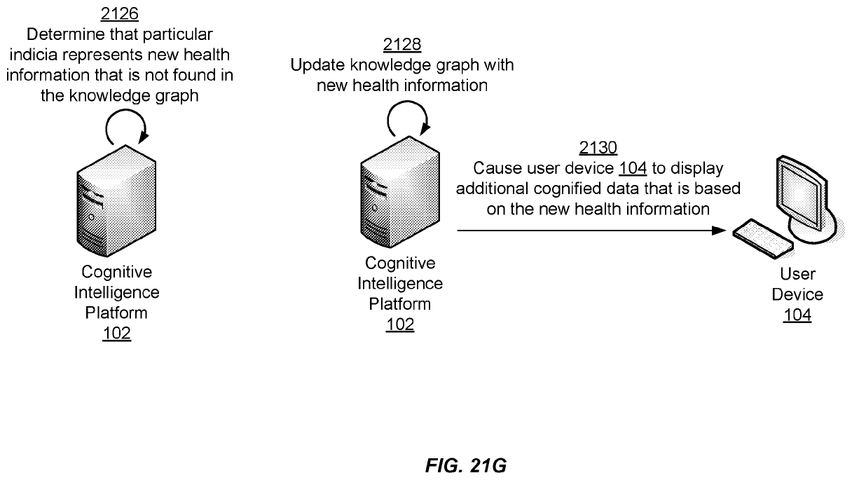

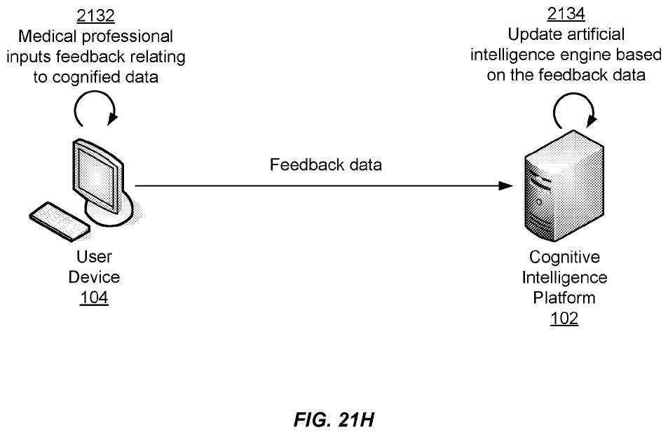

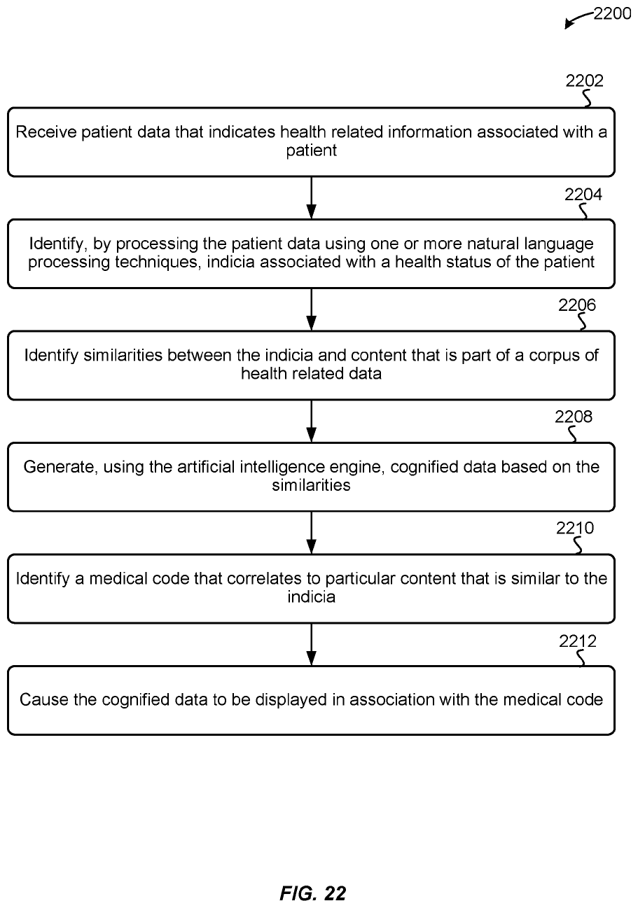

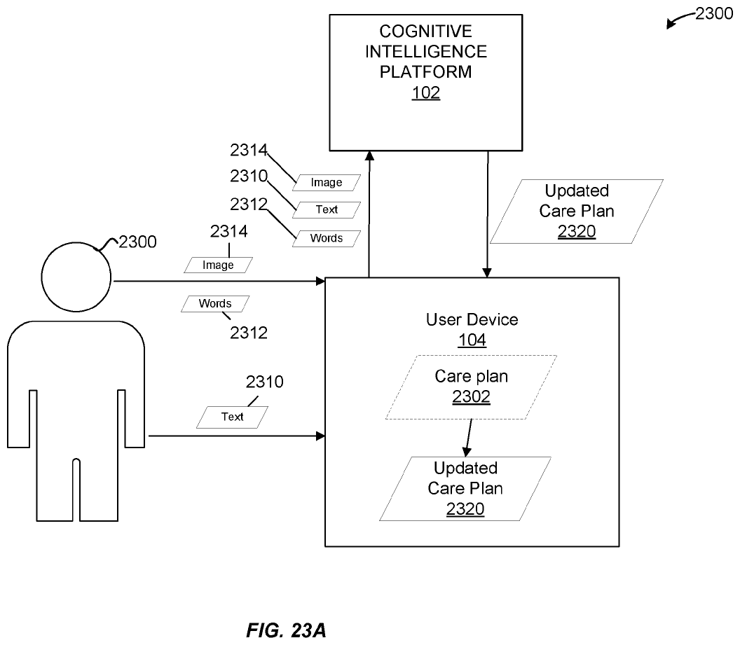

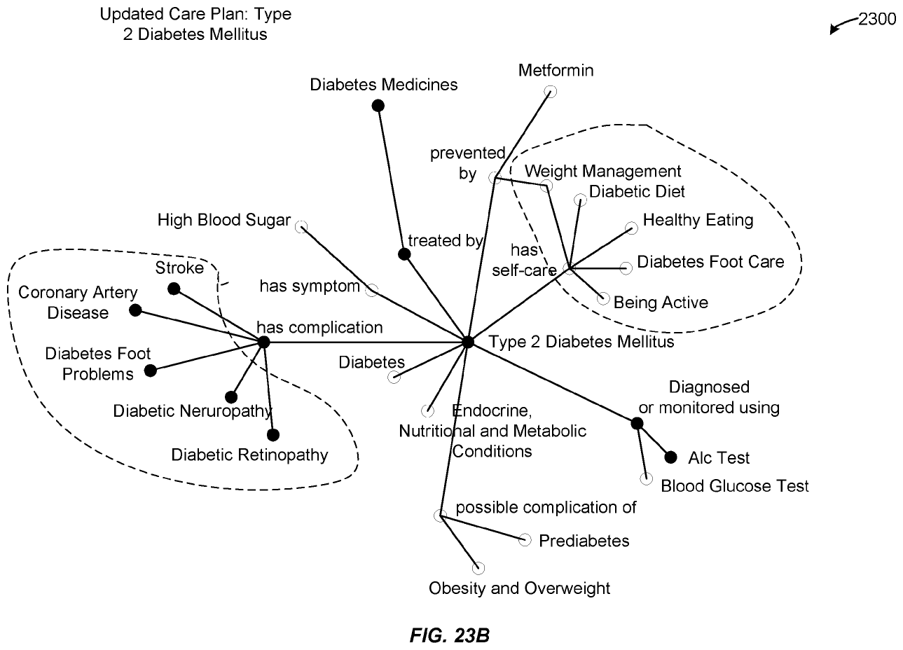

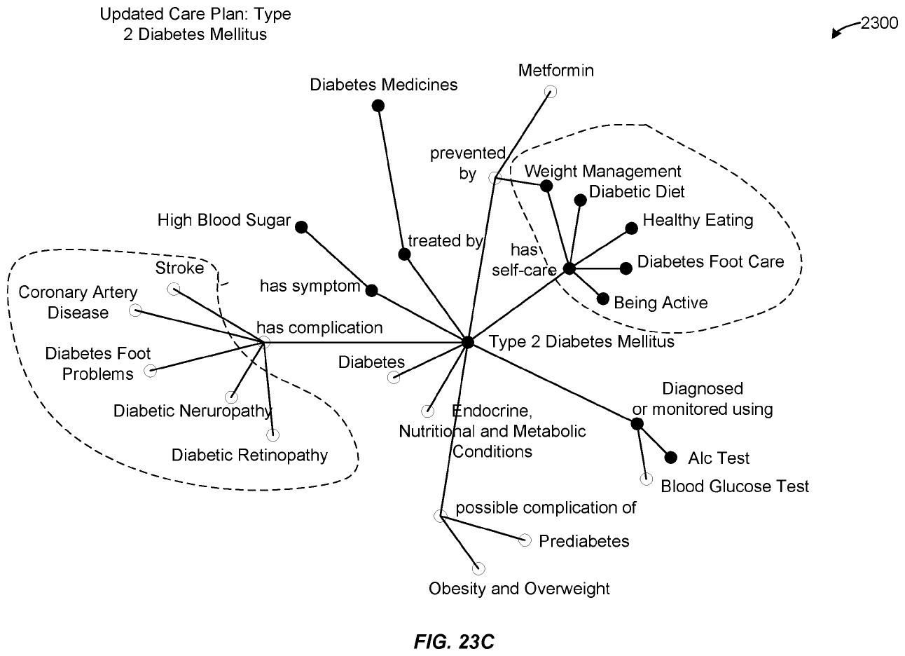

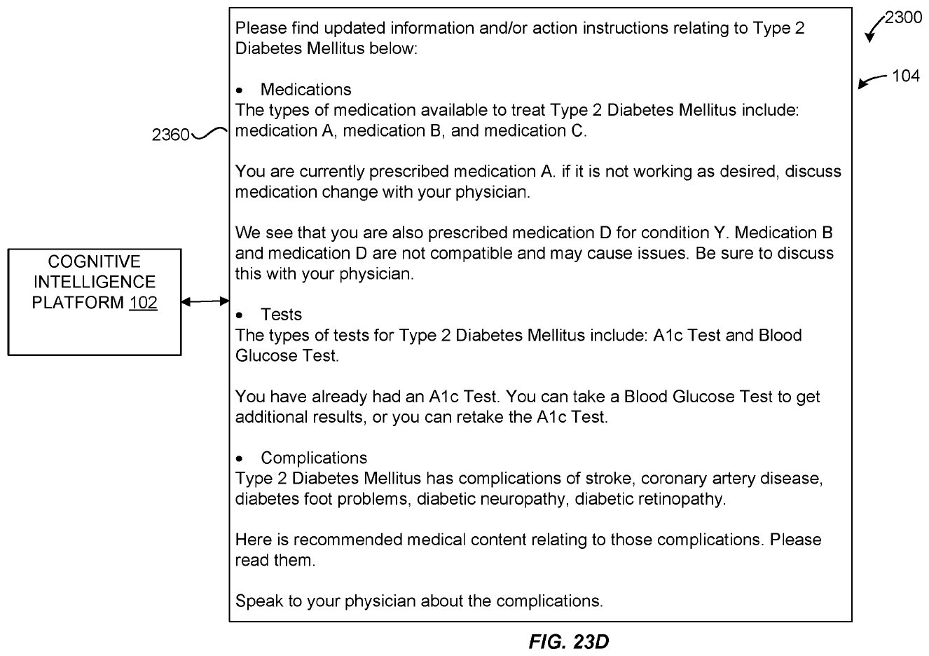

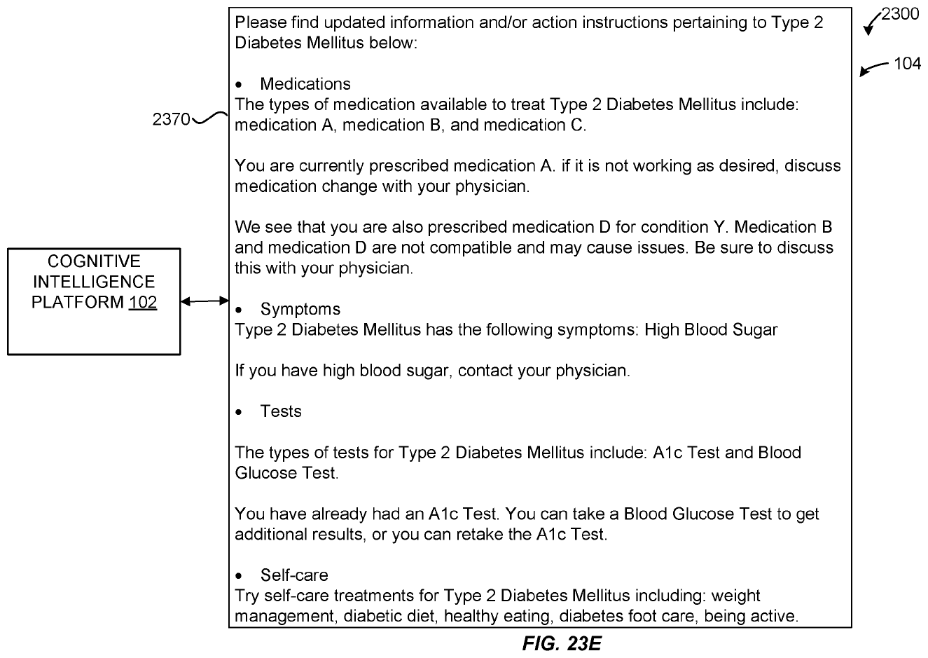

FIGS. 20A-20C show examples for generating a care plan 2050 using a knowledge graph 500 and a patient graph 2000, in accordance with various embodiments. In particular, FIG. 20A depicts the knowledge graph 500 (first data structure) for the medical condition “Type 2 Diabetes Mellitus”. For purposes of explanation, it should be understood that the knowledge graph 500 includes a superset of health artifacts (e.g., elements represented by nodes) pertaining to Type 2 Diabetes Mellitus. The ontological medical data included in the knowledge graph 500 may be maintained by the knowledge cloud 106 and updated based on any changes and/or discoveries regarding medical knowledge of Type 2 Diabetes Mellitus.

FIG. 20B depicts the patient graph 2000 (second data structure) for a particular user having the condition Type 2 Diabetes Mellitus. The patient graph 2000 may also include an engagement profile as metadata that stores interactions of the patient with the various health artifacts presented in a care plan for the user. The interactions may be used to track a level of compliance with the care plan for the user. In some embodiments, the health artifacts represented by the nodes may be added to the patient graph as the patient interacts with the health artifacts. In some embodiments, the health artifacts may be added to the patient graph 2000 if the patient interacts with the health artifact to a threshold level.

As depicted, the patient graph 2000 includes a subset of the superset of health artifacts included in the knowledge graph 500. For example, the patient graph 2000 includes a node representing a “Blood Glucose Test” health artifact that the patient performed. Various information (e.g., result, timestamp, etc.) pertaining to the blood glucose test may be associated with the node. However, the patient graph 2000 does not include a node representing the “A1c” health artifact that is included in the knowledge graph 500 because the patient has not interacted with that health artifact yet. In other words the patient has not performed the A1c test yet.

Other nodes representing health artifacts that are included in the knowledge graph 500 and not in the patient graph 2000 (e.g., due to the patient not interacting with those health artifacts yet) are a node representing “Endocrine, Nutritional and Metabolic Conditions”, a node representing “possible complication of” connected to nodes representing “Prediabetes” and “Obesity and Overweight”, and a node representing “prevented by” connected to a node representing “Metformin”.

To generate the care plan 2050 depicted in FIG. 20C, the cognitive intelligence platform 102 (e.g., the autonomous multipurpose application, the critical thinking engine 108, and/or the knowledge cloud 106) may compare the patient graph 2000 to the knowledge graph 500. Comparing the patient graph 2000 to the knowledge graph 500 may include projecting the patient graph 2000 onto the knowledge graph 500. In some embodiments, projecting the patient graph 2000 onto the knowledge graph 500 may include overlaying the patient graph 500 on the knowledge graph 500, and/or plotting the patient graph 2000 in a same space as the knowledge graph 500. Based on the comparing, the cognitive intelligence platform 102 may select a subset of the superset of health artifacts in the knowledge graph 500. The selecting may be based on identifying nodes representing health artifacts that are included in the knowledge graph 500 and not the patient graph 2000, and/or on areas of the condition the patient selected to manage in FIG. 55. Continuing the example in FIG. 55, the patient selected to manage the areas of “Medications”, “Symptoms”, and “Tests”.

As depicted in FIG. 20C, the care plan 2050 represents the patient graph 2000 projected onto the knowledge graph 500. The nodes that are filled in (black circles) represent health artifacts that are included in the care plan based on the selecting described above. The nodes that are not filled in (empty circles) represent health artifacts that are not included in the care plan 2050. The cognitive intelligence platform 102 selected the node representing “A1c” test to include in the care plan 2050 because the patient graph 2000 included a node representing the blood glucose test and did not include a node representing the A1c test that is included in the knowledge graph 500. Further, the patient selected to manage “Tests”, so including the health artifact A1c test fits that area.

The patient also selected to manage the areas of “Medications” and “Symptoms”. Accordingly, the cognitive intelligence platform 102 included nodes representing health artifacts pertaining to those areas. In particular, the nodes included for the “Symptoms” area are “has symptom” connected to “High Blood Sugar” and the nodes included for the “Medicines” area are “treated by” connected to “Diabetes Medicines”.

Although some nodes are included in the knowledge graph 500 and not in the patient graph 2000, such as the “possible complication of” connected to “Prediabetes” and “Obesity and Overweight” health artifacts, they may not be included in the care plan 2050 because those nodes are associated with areas the patient did not select to manage.

The care plan 2050 may be converted into natural language text by the cognitive intelligence platform 102 using the natural language database 122 according to the techniques disclosed herein. The cognitive intelligence platform 102 may generate action instructions pertaining to the health artifacts included in the care plan 2050.

FIGS. 21A-21H are diagrams of one or more example embodiments described herein. The example embodiment(s) may include the cognitive intelligence platform 102 and the user device 104. As shown in FIGS. 21A-21H, the cognitive intelligence platform 102 may generate cognified data for a claim chart and may cause the user device 104 to display the cognified data in association with a set of related medical codes.

FIG. 21A illustrates an example of a user (e.g., a medical professional) submitting a request for patient data for a patient that has an appointment with a medical professional, in accordance with various embodiments.

In some embodiments, and as shown by reference number 2102, the medical professional may interact with an interface of the autonomous multipurpose application to request existing patient data for a patient. For example, the medical professional may input a patient identifier (e.g., a patient name, a patient medical record number, and/or the like) into a field used to query patient health records, and may submit the request to cause the user device 104 to provide the cognitive intelligence platform 102 with the request. The request may be provided via a communication interface, such as an application programming interface (API) and/or another type of interface.

The existing patient data may be part of a health record for the patient. The health record may include an electronic medical record (EMR), an electronic health record (EHR), a personal health record (PHR), and/or the like. The terms EMR, EHR, and/or PHR may be used interchangeably herein. In some embodiments, the health record may include patient notes taken by medical professionals during previous appointments with the patient. The patient notes may explain symptoms described by the patient or detected by the medical professional, vital signs, recommended treatment, risks, prior health conditions, familial health history, and/or the like.

In some embodiments, the existing patient data may be stored using a database that is accessible to the cognitive intelligence platform 102. For example, the database may be used to store a master dataset of patient data and/or health related data.

In some embodiments, the master dataset of patient data and/or health related data may be organized using a collection of knowledge graphs. A knowledge graph may represent a model that includes individual elements (nodes) and predicates that describe properties and/or relationships between those individual elements. A logical structure (e.g., Nth order logic) may underlie the knowledge graph that uses the predicates to connect various individual elements. The knowledge graph and the logical structure may combine to form a language that recites facts, concepts, correlations, conclusions, propositions, and the like. The knowledge graph and the logical structure may be generated and updated continuously or on a periodic basis by an artificial intelligence engine with evidence-based guidelines, physician research, patient notes in EMRs, physician feedback, and/or the like. The predicates and individual elements may be generated based on data that is input to the artificial intelligence engine. The data may include evidence-based guidelines that is obtained from a trusted source, such as a physician. The artificial intelligence engine may continuously learn based on input data (e.g., evidence-based guidelines, clinical trials, physician research, electronic medical records, etc.) and modify the individual elements and predicates.

For example, a physician may indicate that if a person has a blood sugar level of a certain amount and various other symptoms (e.g., unexplained weight loss, sweating, etc.), then that person has type 2 diabetes mellitus. Such a conclusion may be modeled in the knowledge graph and the logical structure as “Type 2 diabetes mellitus has symptoms of a blood sugar level of the certain amount and various other symptoms,” where “Type 2 diabetes mellitus,” “a blood sugar level of the certain amount,” and “various other symptoms” are individual elements in the knowledge graph, and “has symptoms of” is a predicate of the logical structure that relates the individual element “Type 2 diabetes mellitus” to the individual elements of “a blood sugar level of the certain amount” and “various other symptoms”.

In some embodiments, the cognitive intelligence platform 102 may have codified evidence-based guidelines pertaining to the medical condition to generate the logical structure of the knowledge graph. The generated possible health related information may be a tag that is associated with the indicia in the unstructured data.

In some embodiments, and as shown by reference number 2104, the cognitive intelligence platform 102 may identify the existing patient data. For example, the cognitive intelligence platform 102 may identify the existing patient data by referencing the database using the patient identifier.

In this way, the medical professional is able to use the autonomous multipurpose application to request existing patient data for the patient.

FIG. 21B illustrates an example of the cognitive intelligence platform 102 causing the existing patient data to be displayed on the user device 104, in accordance with various embodiments. In some embodiments, and as shown by reference number 2106, the cognitive intelligence platform 102 may provide the existing patient data to the user device 104. The existing patient data may be provided using the communication interface.

In some embodiments, and as shown by reference number 2108, the user device 104 may display the existing patient data. For example, the user device 104 may display the existing patient data via an interface of the autonomous multipurpose application.

In some embodiments, the user device 104 may present a clinic viewer that displays the existing patient data in a clear, concise, organized format. The clinic viewer may be presented to a medical professional (e.g., doctor, nurse, etc.) and/or an administrator. For example, the existing patient data may be displayed in a group of organized, customizable sections. This allows the medical professional to efficiently and effectively review the patient's information prior to the start of the appointment. As shown as an example, the user device 104 may display the clinic viewer that includes a patient overview section, an appointment summary section, a health record section, a charting section, one or more alerts sections, a medications section, a care plan section, a care team section, an upcoming appointments section, a recommended appointments section, and/or the like.

In this way, the cognitive intelligence platform 102 causes the existing patient data to be displayed in a clear, concise, organized format. This conserves resources (e.g., processing resources, network resources, memory resources, and/or the like) relative to an inferior system that requires the patient data to be obtained from multiple data sources (e.g., by performing multiple queries), that requires the medical professional to open and navigate through numerous screens in order to view all of the patient data, that presents the existing patient data ineffectively and/or inefficiently, and/or the like.

FIG. 21C illustrates an example of the user device 104 generating and providing new patient data to the cognitive intelligence platform 102, in accordance with various embodiments. For example, and as shown by reference number 2110, the user device 104 may generate new patient data during the appointment. For example, during the appointment with the patient, the medical professional may input patient notes into one or more fields of the patient chart interface.

As a specific example, the patient may begin describing a medical situation to the medical professional. To create patient notes for the appointment, the medical professional may first select a “new chart” button that may be found on the charting tab of the patient profile interface that displays the existing patient data (e.g., shown in the interface depicted in FIG. 21B). This may cause the user device 104 to display a patient chart interface that allows the medical professional to input patient notes relating to the patient's medical situation. In some embodiments, the medical professional may input free-form text (e.g., patient notes), may select a descriptor of the patient's medical situation from a drop-down menu, may upload a file, and/or the like.

In the example shown, the medical professional may provide, as input to the clinical summary portion of the patient chart interface, “Mrs. N reports increasing problems with frontal headaches over the past 3 months. These are usually bi-frontal, throbbing, and mild to moderately severe. She has missed work on several occasions because of associated nausea and vomiting.” The right hand side of the patient chart interface may be populated in real-time using medical codes and/or cognified data, as will be described further herein.

In some embodiments, the user device 104 may capture the new patient data. For example, the user device 104 may capture the new patient data by generating a recording of a conversation between the patient and the medical professional. The recording may be an audio recording, a video recording, and/or the like.

In some embodiments, the user device 104 may generate the recording using one or more features of the autonomous multipurpose application. In some embodiments, the user device 104 may generate the recording using an application capable of communicating with the autonomous multipurpose application (e.g., using an API). In some embodiments, the recording may be generated by another device (e.g., external to the user device 104) and the other device may be configured to communicate with the user device 104 and/or the cognitive intelligence platform 102. In some embodiments, the user device 104 may provide the recording to the cognitive intelligence platform 102 for further processing. In some embodiments, the user device 104 may perform one or more processing actions that are described below as being performed by the cognitive intelligence platform 102.

In some embodiments, the cognitive intelligence platform 102 may generate, as part of the new patient data, a transcript of an audio portion of the recording. For example, the cognitive intelligence platform 102 may generate the transcript using an audio-to-text conversion technique. In some embodiments, this technique may be used when the medical professional dictates the new patient data to the user device 104 using a microphone included in the user device 104 and/or records a video using a camera and the microphone included in the user device 104.

Additionally, or alternatively, the cognitive intelligence platform 102 may generate, as part of the new patient data, tone data that indicates a tone of the patient, emotion data that indicates an emotion of the patient, movement data that indicates a movement or gesture of the patient, and/or the like. For example, the cognitive intelligence platform 102 may process a video recording using a machine learning model that has been trained to identify patterns between images of certain facial expressions, certain body language, certain emotions (e.g., happy, angry, sad, etc.), certain tones of voice, and/or the like. In this case, the cognitive intelligence platform 102 may generate the tone data, the emotion data, the movement data, and/or the like, by using the machine learning model to perform a facial recognition technique, a target identification technique, an image recognition and/or matching technique, a sentiment analysis technique, and/or the like. Additional information regarding detecting tone of the patient, emotion of the patient, and/or the like, may be found in connection with FIGS. 23A-23E.

In some embodiments, and as shown by reference number 2112, the user device 104 may provide the new patient data to the cognitive intelligence platform 102. The new patient data may be provided via the communication interface.

In some embodiments, the user device 104 may be configured to periodically (e.g., every five minutes, once an hour, and/or the like) provide the cognitive intelligence platform 102 with new patient data input by the medical professional. In some embodiments, the user device 104 may be configured to immediately provide the cognitive intelligence platform 102 with new patient data. As will be shown further herein, this allows the cognitive intelligence platform 102 to quickly analyze the new patient data and to provide the medical professional with cognified data that may assist the medical professional in performing tasks during (and/or after) the appointment with the patient. A task may include diagnosing the patient, providing the patient with a medical opinion and/or a recommendation, scheduling a follow-up appointment, prescribing medication, and/or the like.

In this way, the user device 104 captures and provides the cognitive intelligence platform 102 with the new patient data.

FIG. 21D illustrates an example of the cognitive intelligence platform 102 identifying indicia and identifying similarities between the indicia and content included in the corpus of health data, in accordance with various embodiments. In some embodiments, and as shown by reference number 2114, the cognitive intelligence platform 102 may process the new patient data using natural language processing techniques to identify indicia. For example, the patient notes indicated by the patient data may include numerous strings of characters arranged into sentences and the cognitive intelligence platform 102 may process the sentences using natural language processing techniques to identify the indicia. The natural language processing techniques may include receiving the patient data including a stream of Unicode characters and converting the character stream into a sequence of indicia (lexical items, words, phrases, and syntactic markers) that may be used to understand the content of the patient data, as described further below.

The indicia may be associated with a health status of the patient. The indicia may include predicates, objectives, nouns, verbs, cardinals, ranges, keywords, phrases, numbers, concepts, and/or the like. The natural language processing techniques may include one or more syntax-based techniques and/or one or more semantic-based techniques, such as a parts of speech tagging technique, a parsing technique, a lemmatization and/or stemming technique, a named entity recognition (NER) technique, a sentiment analysis technique, and/or the like.

Additionally, or alternatively, the cognitive intelligence platform 102 may identify indicia using artificial intelligence engine 109. For example, the artificial intelligence engine 109 may be trained to identify the indicia in text based on commonly used indicia pertaining to the possible medical condition. In this case, the artificial intelligence engine 109 may determine commonly used indicia for various medical conditions based on evidence-based guidelines, clinical trial results, physician research, and/or the like, that are input to one or more machine learning models.

In some embodiments, and as shown by reference number 2116, the cognitive intelligence platform 102 may generate tags for the indicia. For example, tags corresponding to possible health related information may be generated and associated with the indicia, such that a logical structure is assigned to the unstructured data. As a specific example, the tags may specify “A leads to B” (where A is a health related information and B is another health related information), “B causes C” (where C is yet another health related information), “C has complications of D” (where D is yet another health related information), and/or the like. Tags may, for example, be generated based a comparison of the indicia and the content included in the corpus of health related data.

In this way, the cognitive intelligence platform 102 identifies indicia and generates tags that serve as a way to map particular indicia to particular content represented in a knowledge graph, tags that may be used to identify content that is structurally similar to the indicia (as further described below), and/or the like.

FIG. 21E provides an illustration of an example for identifying similarities between the indicia and content included in a corpus of health data and generating cognified data based on the identified similarities. In some embodiments, and as shown by reference number 2118, the cognitive intelligence platform 102 may identify similarities between the indicia and content stored using one or more knowledge graphs. For example, the cognitive intelligence platform 102 may identify similarities between characteristics of the indicia and content characteristics of the content. Content, as used herein, may refer to elements (e.g., nodes) of the one or more knowledge graphs, predicates (e.g., edges) of the one or more knowledge graphs, and/or the like. The cognitive intelligence platform 102 may identify the similarities by using the artificial intelligence engine 109 to compare the characteristics of the indicia with the content characteristics of the content, as further described below.

The characteristics and/or the content characteristics may include characteristics relating to semantic meanings, characteristics associated with a semantic relatedness, characteristics associated with a logical structural, and/or the like. The identifiable similarities may include semantic similarities, semantically-related similarities, structural similarities, and/or the like.

In some embodiments, the cognitive intelligence platform 102 may identify a first set of similarities between the indicia and elements and/or predicates of the knowledge graph. For example, the cognitive intelligence platform 102 may compare the indicia with elements and/or predicates of the knowledge graph. If a particular indicia satisfies a threshold level of similarity with a particular element and/or predicate of the knowledge graph, the cognitive intelligence platform 102 may identify the compared items as being similar. A measured level of similarity may be based on a semantic similarity between the compared items, a semantic relatedness between the compared items, and/or the like.

Additionally, or alternatively, the cognitive intelligence platform 102 may identify a structural similarity between a logical structure of the indicia and a logical structure of particular content of a knowledge graph. For example, the cognitive intelligence platform 102 may have generated a data structure that associates respective indicium included in the indicia. The data structure may be a patient graph, a collection of tags that have a logical structure, and/or the like. Next, the cognitive intelligence platform 102 may compare a logical structure of the indicia with a logical structure of the content and may identify a structural similarity between the logical structure of the indicia and the logical structure of the content (e.g., a known predicate of the logical structure) based on the comparison. As a specific example, if the logical structure of the indicia forms a sentence stating “Patient X has symptoms of High Blood Pressure” and the logical structure of a portion of the knowledge graph (e.g., content) forms a sentence stating “Type 2 Diabetes has symptoms of High Blood Pressure,” and the logical structures match or satisfy a threshold level of similarity with each other, then the cognitive intelligence platform 102 may identify the logical structure of the indicia and the logical structure of the portion of the knowledge graph as being structurally similar.

In some embodiments, and as shown by reference number 2120, the cognitive intelligence platform 102 may generate the cognified data based on the identified similarities. For example, the cognitive intelligence platform may have trained one or more machine learning models (e.g., as part of the artificial intelligence engine 109) to transform unstructured input data (e.g., patient notes, and/or the like) into cognified data using the one or more knowledge graphs and their respective logical structures. The structural similarity between possible health related information and a known predicate may enable identifying a pattern, such as a treatment pattern, an education and content pattern, an order pattern, a referral pattern, a quality of care pattern, a risk adjustment pattern, and/or the like. The one or more machine learning models may generate the cognified data based on the structural similarity, the pattern identified, and/or the like. Accordingly, the machine learning models may use a combination of knowledge graphs, logical structures, structural similarity comparison mechanisms, and/or pattern recognition to generate the cognified data. The cognified data may, in some cases, be output by the one or more trained machine learning models. In other cases, the cognified data may be generated based on scores output by the one or more trained machine learning models.

A pattern may be detected by identifying structural similarities between the tags and the logical structure in order to generate the cognified data. The pattern may pertain to treatment, quality of care, risk adjustment, orders, referral, education and content patterns, and/or the like. The structural similarity and/or the pattern may be used to cognify the corpus of data. Cognification may refer to instilling intelligence into something. In the present disclosure, unstructured data may be cognified into cognified data by instilling intelligence into the unstructured data using the knowledge graph and the logical structure. Cognified data may include a summary of a health related condition of a patient, where the summary includes insights, conclusions, recommendations, identified gaps (e.g., in treatment, risk, quality of care, guidelines, etc.), and/or the like.

Cognified data, as used herein, may provide a summary of the medical condition of the patient, where the summary includes insights, conclusions, recommendations, identified gaps (e.g., in treatment, risk, quality of care, guidelines, etc.), and/or the like. The summary of the medical condition may include one or more insights not present in the unstructured data. In some embodiments, the summary may identify gaps in the unstructured data, such as treatment gaps (e.g., should prescribe medication, should provide different medication, should change dosage of medication, etc.), risk gaps (e.g., the patient is at risk for cancer based on familial history and certain lifestyle behaviors), quality of care gaps (e.g., need to check-in with the patient more frequently), and/or the like. Additionally, or alternatively, the summary of the medical condition may include one or more conclusions, recommendations, complications, risks, statements, causes, symptoms, and/or the like, pertaining to the medical condition. Additionally, or alternatively, the summary of the medical condition may indicate another medical condition that the medical condition can lead to. Accordingly, the cognified data represents intelligence, knowledge, and logic cognified from unstructured data.

In some embodiments, the cognified data generated by the cognitive intelligence platform 102 may include a patient graph. For example, the cognitive intelligence platform 102 may use a machine learning model to generate the patient graph. In some embodiments, the patient graph may be generated in real-time. The patient graph may include elements (e.g., health artifacts) and branches representing relationships between the elements. The elements may be represented as nodes in the patient graph. The elements may represent interactions and/or actions the user has had and/or performed pertaining to the condition. For example, if the condition is diabetes and the user has already performed a blood glucose test, then the user may have a patient graph corresponding to diabetes that includes an element for the blood glucose test. The element may include one or more associated information, such as a timestamp of when the blood glucose test was taken, whether it was performed at-home or at a care provider, a result of the blood glucose test, a medical code representing the blood glucose test, and/or the like.

Typically, a medical coder may be given the patient chart completed by the medical professional and may analyze the patient chart and assign medical codes to aspects of the patient chart using a classification system. The medical codes may be stored using a data structure that maps respective medical codes with corresponding supplemental health related information. However, the medical professional is often unable to utilize quick access to the supplemental health related information because the medical codes are not created until after the patient chart has been completed.

In some embodiments, and as shown by reference number 2122, the cognitive intelligence platform 102 may identify (or generate) medical codes relating to the health status or condition of the patient. In some embodiments, the cognitive intelligence platform 102 may identify a medical code that correlates to the content having the content characteristics similar to the characteristics of the tags that were generated. In some embodiments, the cognitive intelligence platform 102 may identify (or generate) medical codes that map to specific identified indicia. The cognitive intelligence platform 102 may be configured to identify (or generate) one or more medical codes for each respective identified indicia. For example, if the indicia specifies “frontal headaches,” and a knowledge graph specifies different types of headaches, the cognitive intelligence platform may identify a medical code corresponding to each respective type of headache that is specified in the knowledge graph. The knowledge graph may be used to store the medical codes as metadata associated with respective nodes (e.g., nodes corresponding to different types of headaches). In some embodiments, a lookup table may be used that stores indicia and corresponding medical codes. If a medical code has not yet been created, the cognitive intelligence platform 102 may generate the medical code and submit it to a medical coder for review and approval.

In this way, the cognitive intelligence platform 102 generates cognified data that can be used to assist the medical professional with providing proper medical care to the patient, as will be shown further herein.

FIG. 21F illustrates an example of the cognitive intelligence platform 102 causing the user device 104 to display the cognified data in association with the medical codes, in accordance with various embodiments. In some embodiments, and as shown by reference number 2124, the cognitive intelligence platform 102 may cause the user device 104 to display the cognified data in association with the medical codes. The cognified data may be displayed in association with the medical codes via the patient chart interface of the autonomous multipurpose application.

In the example shown, the user device 104 may display the patient chart for Zahra Smith. The top half of the right hand side of the interface may include medical codes relating to headaches. The medical codes may include codes that are part of a medical classification list belonging to the International Statistical Classification of Diseases and Related Health Problems (ICD) (shown as ICD 10 Codes) and codes that are part of a Systematized Nomenclature of Medicine (SNOMED) (shown as SNOMED Codes).

Specifically, the ICD 10 Codes include G44.001 and G44.009. G44 is a code for cluster headache syndrome and 001 and 009 are codes representing varied levels of severity of the syndrome (e.g., intractable, not intractable, etc.). The SNOMED codes include 103011009 and 121021000119105. 103011009 is a code for a benign exertional headache and 121021000119105 is a code for a new daily persistent headache. The interface also includes buttons that allow the medical professional to select “YES” or “NO” based on whether a given medical code is applicable to the patient. Additionally, the interface includes an export button to allow the medical professional to create a portable document format (PDF) of the patient chart or any suitable file format for representing the patient chart.

Continuing with the example, the bottom half of the right hand side of the interface may include cognified data that is separated by sections, such as a Quality Alerts section, an Education section (e.g., to be recommended for the patient), a Care Plans section, and/or the like. Specifically, the Quality Alerts section may include a first field with text stating “Patient with uncontrolled severe headaches who has not been referred to a neurologist” and a second field with text stating “Select to read recommended materials to educate patient on headaches.”

In some embodiments, the cognitive intelligence platform 102 may cause the medical codes to be displayed (and not the cognified data). Additionally, or alternatively, the cognitive intelligence platform 102 may cause the cognified data to be displayed (and not the medical codes).

In some embodiments, the cognitive intelligence platform 102 may cause the medical codes to be displayed at a first time and the cognified data to be displayed at a second time. For example, as the medical professional begins to input patient notes (e.g., a clinical summary), a first set of patient data may be provided to the cognitive intelligence platform 102. If the medical professional has yet to provide sufficient patient data needed to generate meaningful cognified data, the cognitive intelligence platform 102 may simply identify the medical codes that map to the identified indicia (e.g., using a lookup table) and may cause the medical codes to be displayed (e.g., at the first time). As the medical professional continues to input additional patient notes, a second set of patient data may be provided to the cognitive intelligence platform 102. This may allow the cognitive intelligence platform 102 to generate cognified data (and/or to identify any additional relevant medical codes) and to cause the cognified data (and/or any additional relevant medical codes) to be displayed (e.g., at the second time) with the associated medical codes. The associated medical codes may be identified based on being correlated to content in a knowledge graph having similar characteristics to the characteristics of the tags, indicia, or some combination thereof.

In some embodiments, the cognitive intelligence platform 102 may cause the cognified data to be displayed in association with the medical codes in real-time or near real-time. As discussed herein, the terms “real-time” or “near real-time” may refer to performing an action in less than two seconds after a triggering event occurs. Real-time may be relative to a time at which the cognitive intelligence platform 102 has identified similarities between the indicia and the content of the knowledge graph, relative to a time at which the cognitive intelligence platform 102 has generated tags for the indicia, relative to a time when the patient data is received, and/or the like. In some embodiments, the triggering event may include receiving the patient data from the user device 104 of the medical professional.

In some embodiments, the cognitive intelligence platform 102 may cause a patient graph to be displayed by the user device 104. For example, the cognitive intelligence platform 102 may generate a patient graph. The patient graph may include a set of nodes and a set of edges. The set of nodes may include various patient data, such as demographic information of the patient, patient notes of the medical professional, procedures involving the patient, labs and vitals for the patient, medications of the patient, a care plan for the patient, and/or the like. The set of edges may include predicates or relationships between particular patient data. The cognitive intelligence platform 102 may cause the patient graph to be displayed via an interface of the autonomous multipurpose application, such that the medical professional may use the patient graph as supplemental visual aid during the appointment, after the appointment, and/or the like. In some embodiments, the patient graph may be presented in natural language, graph form, and/or any other suitable representation. In some embodiments, such as when the patient graph is generated before the appointment, the cognitive intelligence platform 102 may simply update the patient graph with the patient notes that are being input by the medical professional during the appointment.

In this way, the cognitive intelligence platform 102 causes the user device 104 to display, in real time or near real-time, the patient data and/or the cognified data in association with the medical codes. By displaying the cognified data, the medical codes, and/or associations between them, the cognitive intelligence platform 102 allows the medical professional to view relevant suggestions that may be considered when developing a medical opinion regarding the health or condition of the patient. This improves the quality of healthcare service provided by the medical professional. Additionally, resources (e.g., processing resources, network resources, memory resources, and/or the like) are conserved by eliminating the need to generate, transmit and/or store duplicative health related information. For example, the medical professional might otherwise upload a patient chart that includes health related information of the patient that is already stored by a backend server, a medical coder might create a duplicative medical code (e.g., if different language or wording is used by the medical professional), and/or the like. Furthermore, the cognitive intelligence platform 102 reduces a utilization of resources of a medical coding device that a medical coder would otherwise have to use to identify and/or generate the medical codes.

FIG. 21G illustrates an example for identifying missing information in the corpus of health related data, in accordance with various embodiments. In some embodiments, and as shown by reference number 2126, the cognitive intelligence platform 102 may determine that particular indicia represent new health related information that is not found in the corpus of health related data. For example, the cognitive intelligence platform 102 (e.g., using the artificial intelligence engine) may identify at least one piece of information missing in the corpus of health related data for the patient using the cognified data. The at least one piece of information pertains to a treatment gap, a risk gap, a quality of care gap, and/or the like.

In some embodiments, and as shown by reference number 2128, the cognitive intelligence platform 102 may generate additional cognified data and update the corpus of health related data with the new health related information. The corpus of health related data may be updated by adding one or more nodes and edges to a knowledge graph. For example, a node may represent the new health related information and one or more connecting edges may represent predicates or relationships between the new health related information and existing health related information.

In some embodiments, and as shown by reference number 2130, the cognitive intelligence platform 102 may cause the user device 104 to display additional cognified data that is based on the new health related information and/or existing/new associated medical codes. For example, the cognitive intelligence platform 102 may generate additional cognified data based on the new health related information and may cause the additional cognified data to be displayed by user device 104 with the existing/new associated medical codes.

The additional cognified data may, for example, be a notification that includes a recommendation based on the new health related information. For example, if certain symptoms are described for the patient in the corpus of health related data and those symptoms are known to result from a certain medication currently prescribed to the patient, but the corpus of health related data does not indicate switching medications, then the new health related information may represent a treatment gap. Consequently, the cognitive intelligence platform 102 may generate a recommendation to switch medications to one that does not cause those symptoms. In some embodiments, the recommendation may be stored as part of the corpus of health related data (e.g., in association with the new health related information and/or other related elements and/or predicates of a knowledge graph).

In this way, the cognitive intelligence platform 102 uses artificial intelligence to identify new health related information that is missing from the corpus of health related data, generates additional cognified data based on the new health related information, and causes the additional cognified data and/or associated medical codes to be displayed by the user device 104.

FIG. 21H illustrates an example of using feedback pertaining to the accuracy of cognified data to update the artificial intelligence engine, in accordance with various embodiments. In some embodiments, and as shown by reference number 2130, the medical professional may interact with an interface of the autonomous multipurpose application to input feedback relating to the cognified data. This may cause feedback data for the feedback to be provided to the cognitive intelligence platform 102.

For example, the physician may be presented with the cognified data including associated medical codes and may review the cognified data including associated medical codes in the user interface presenting the intelligent chart in FIG. 21F. The physician may be presented with options to verify the accuracy of portions or all of the cognified data for the particular patient. For example, the physician may select a first graphical element (e.g., button, checkbox, and/or the like) next to portions of the cognified data that are accurate and may select a second graphical element next to portions of the cognified data that are inaccurate. If the second graphical element is selected, an input box may appear and a notification may be presented to provide a reason why the portion is inaccurate and to provide corrected information. The feedback may be provided to the cognitive intelligence platform 102.

In some embodiments, and as shown by reference number 2134, the cognitive intelligence platform 102 may update the artificial intelligence engine based on the feedback data. For example, a closed-loop feedback system may be implemented using these techniques. The feedback may enhance the accuracy of the cognified data as the artificial intelligence engine continues to learn and improve. The cognitive intelligence platform 102 may update the artificial intelligence engine by retraining one or more machine learning models based on the feedback data. For example, if a machine learning model is a neural network, the cognitive intelligence platform 102 may retrain the neural network by modifying one or more weights, such that the neural network is able to accurately score subsequently received input data in a manner that reflects the feedback.

In this way, the cognitive intelligence platform 102 ensures that subsequently generated cognified data is accurate. This improves the overall healthcare service provided to the patient, conserves resources that might otherwise be wasted generating inaccurate cognified data, and/or the like.

As indicated above, FIGS. 21A-21H are provided merely as an example. Other examples are possible and may differ from what was described with regard to FIGS. 21A-21H. For example, there may be additional devices and/or networks, fewer devices and/or networks, different devices and/or networks, or differently arranged devices and/or networks than those shown in FIGS. 21A-21H. Furthermore, two or more devices shown in FIGS. 21A-21H may be implemented within a single device, or a single device shown in FIGS. 21A-21H may be implemented as multiple, distributed devices. Additionally, or alternatively, a set of devices (e.g., one or more devices) of the one or more example embodiments described above may perform one or more functions described as being performed by another set of devices of the one or more example embodiments.

FIG. 22 shows a method 2200 for generating cognified data and causing the cognified data to be displayed in association with related medical codes, in accordance with various embodiments. In some embodiments, the method 2200 is implemented on a cognitive intelligence platform. In some embodiments, the cognitive intelligence platform is the cognitive intelligence platform 102 as shown in FIG. 1. In some embodiments, the cognitive intelligence platform 102 is implemented on the computing device 600 shown in FIG. 6. The method 2200 may include operations that are implemented in computer instructions stored in a memory and executed by a processor of a computing device.

At block 2202, the method 220 may include receiving patient data that indicates health related information associated with a patient. For example, the computing device 1400 (e.g., using may receive patient data that indicates health related information associated with a patient, as described above.

At block 2204, the method 2200 may include identifying, by processing the patient data using one or more natural language processing techniques, indicia associated with a health status of the patient. For example, the computing device 1400 may identify, by processing the patient data using one or more natural language processing techniques, indicia associated with a health status of the patient, as described above.

At block 2206, the method 2200 may include identifying similarities between the indicia and content that is part of a corpus of health related data. For example, the computing device 1400 may identify similarities between characteristics of the indicia and content characteristics for the content, as described above.

In some embodiments, the computing device 1400 may compare the indicia with the content, where the content is stored using a knowledge graph, and may identify a semantic or semantically-related similarity between a characteristic of the indicia and a corresponding content characteristic. In some embodiments, the computing device 1400 may compare the indicia with the content, where the content is stored using a knowledge graph. In some embodiments, the computing device 1400 may identify, using a logical structure, a structural similarity of the indicia and a known predicate of the logical structure of the knowledge graph.

At block 2208, the method 2200 may include generating, using an artificial intelligence engine, cognified data based on the similarities. For example, the computing device 1400 may generate, using an artificial intelligence engine, cognified data based on the similarities, as described above. The cognified data may provide a summary of the health status for the patient and may include at least one of: a conclusion, a recommendation, a complication, a risk statement, a description of a cause of a health complication, or a description of symptoms of the health complication.

In some embodiments, the computing device 1400 may generate the cognified data based on the semantic or semantically-related similarity. Additionally, or alternatively, the computing device 1400 may generate the cognified data based on the structural similarity.

In some embodiments, the computing device 1400 may identify, using the artificial intelligence engine, a pattern based on a structural similarity between a logical structure of a patient graph used to store the indicia and a logical structure of a knowledge graph used to store the content. The computing device 1400 may generate the cognified data based on the pattern. In some embodiments, the computing device 1400 may identify, using the artificial intelligence engine, a pattern based on a structural similarity between a logical structure of a data structure associated with the indicia and a logical structure of a knowledge graph used to store the content. The data structure may be represented using a collection of tags generated by the computing device 1400, by a patient graph, and/or the like. The computing device 1400 may generate the cognified data based on the pattern. In some embodiments, the computing device 1400 may generate the cognified data in real-time or near real-time relative to receiving the health related information.

At block 2210, the method 2200 may include identifying a medical code that correlates to particular content that is similar to the indicia. For example, the computing device 1400 may identify a medical code that correlates to particular content characteristics of the content that are similar to the characteristics of the indicia.

At block 2212, the method 2200 may include causing the cognified data to be displayed in association with the medical code. For example, the computing device 1400 may cause the cognified data to be displayed in association with medical code, as described above.

In some embodiments, the computing device 1400 may determine, using the cognified data, that particular indicia represents new health information that is not found in the corpus of health related data. Consistent with the above disclosure, the examples of systems and method enumerated in the following clauses are specifically contemplated and are intended as a non-limiting set of examples.

FIGS. 23A-23E show examples of modifying a care plan based on a detected emotion of the patient, a detected tone of the patient, a different medical outcome entered by a physician, or some combination thereof, in accordance with various embodiments. FIG. 23A depicts a user 2300 (e.g., patient) using the user device 104. The cognitive intelligence platform 102 provided a care plan 2302 that was originally generated for the patient for a medical condition of the patient. The care plan 2302 may include an action instruction pertaining to the medical condition of the user 2300, such as an instruction to read certain recommended content for the medical condition, schedule an appointment with a physician, perform a certain test for the medical condition, etc.

When the care plan 2302 is presented to the user via display of the user device 104, the user device 104 may receive various input data from the user 2300. For example, the user may enter text 2310 using any suitable input peripheral (e.g., mouse, keyboard, touchscreen) of the user device 104, the user may speak words 2312 that a microphone of the user device 104 receives, and/or the user device 104 may capture an image 2314 (e.g., still-image, series of images, video) of the user's face and/or body using a camera of the user device 104. The input data 2310, 2312, and/or 2314 may be transmitted by the user device 104 to the cognitive intelligence platform 102.

The cognitive intelligence platform 102 may process the input data to detect a tone of the user 2300 and/or an emotion of the user 2300. For example, a machine learning model may be trained on training data that identifies patterns between images 2314 of certain facial expressions/body language and certain emotions (e.g., happy, angry, sad, etc.). In that regard, facial recognition techniques may be used, such as detecting the face and/or body, scanning the face and/or body, creating targets, matching the targets, and verifying. The machine learning model may receive the image 2314 of the user 2300 as input and output the emotion of the user 2300. Further, spoken words 2312 and/or the text 2310 may be processed by a machine learning model that is trained on training data that identifies patterns between the spoken words and/or text and certain emotions and/or tones (e.g., attitude of the user 2300 towards the subject presented on the user device 104). The tones may include cheerful, pessimistic, optimistic, sarcastic, hostile, and the like. The machine learning model may use certain natural language processing techniques disclosed herein.

In some embodiments, the input data 2310, 2312, and/or 2314 may be received by the cognitive intelligence platform 102 when the care plan 2302 is presented on the user device 104. In some embodiments, the input data 2310, 2312, and/or 2314 may be received by the cognitive intelligence platform 102 at any time the user is using the user device (e.g., even if the user is not logged into or using the autonomous multipurpose application of the cognitive intelligence platform 102.

If the cognitive intelligence platform 102 receives the input data 2310, 2312, and/or 2314 when the care plan 2302 is presented to the user 2300 on the user device 104, and the cognitive intelligence platform 102 detects a negative emotion (e.g., angry) and/or tone (e.g., hostile), the cognitive intelligence platform 102 may modify the care plan 2302 to generate an updated care plan 2320. The updated care plan 2320 may include a different subset of health artifacts than the care plan 2302. The different subset of health artifacts may be selected based on various criteria. For example, the different subset of health artifacts may be selected from a knowledge graph as long as the different subset of health artifacts includes a randomly selected health artifact that was not included in the care plan 2302.

In some embodiments, the different set of health artifacts in the updated care plan 2320 may be selected based on the detected tone and/or emotion. For example, a machine learning model may be trained to generate updated care plans based on training data that includes care plans that have historically improved a users' tone and/or emotion. That is, the machine learning model may be trained to receive a care plan, detected emotion, and/or detected tone, and to generate an updated care plan using the care plan, detected emotion, and/or detected tone based on certain health artifacts of the medical condition that are not included in the care plan and that have historically improved the current emotion and/or tone of the user 2300. Accordingly, the cognitive intelligence platform 102 may track the detected conditions and/or tones of the users in reaction to care plans that are presented on the user device 104.

In some embodiments, if the detected emotion (e.g., happy) and/or tone (e.g., cheerful) is positive, the cognitive intelligence platform 102 may modify the care plan to generate an updated care plan 2320. The updated care plan 2320 may include a different subset of health artifacts than the care plan 2302. The different subset of health artifacts may be selected based on various criteria. For example, the different subset of health artifacts may be selected from a knowledge graph as long as the different subset of health artifacts includes a randomly selected health artifact that was not included in the care plan 2302.

In some embodiments, the different set of health artifacts in the updated care plan 2320 may be selected based on the detected tone and/or emotion. For example, if the detected tone and/or emotion is positive, a machine learning model may be trained to generate updated care plans that include health artifacts with which the user 2300 is likely to interact due to the positive tone and/or emotion. A machine learning model may be trained to receive a care plan, detected emotion, and/or detected tone, and to generate an updated care plan using the care plan, detected emotion, and/or detected tone based on certain health artifacts of the medical condition that are not included in the care plan and that have historically shown a likelihood of being interacted with by the user 2300 when the user 2300 exhibits the positive emotion and/or tone.

Further, the cognitive intelligence platform 102 may receive the input data 2310, 2312, and/or 2314 at any time the user is using the user device 104. The cognitive intelligence platform 102 may use a machine learning model trained to output certain updated care plans 2320 based on the detected emotion and/or tone of the user 2300 based on the received input data 2310, 2312, and/or 2314. For example, if the cognitive intelligence platform 102 detects the user has an angry emotional state, the cognitive intelligence platform 102 may use a machine learning model trained to include certain health artifacts in an updated care plan 2320 that historically improve the emotional state of the user 2300.

FIG. 23B depicts an example updated care plan 2320.1. For purposes of explanation, the original care plan 2302 was the care plan 2350 depicted in FIG. 57C. The care plan 2350 may have been presented in the patient viewer on the user device 104 and included the information pertaining to “Symptoms” area. Input data, such as the image 2314 (e.g., face image, body image), may be received by the cognitive intelligence platform 102 and processed. The cognitive intelligence platform 102 may input the image 2314 into the machine learning model trained to detect an emotion and/or tone of the user 2300 based on a facial expression and/or body language of the user 2300 in the image 2314.

The cognitive intelligence platform 102 may determine the user 2300 experienced a negative emotion (e.g., angry) when viewing the “Symptoms” area of the care plan 2350. Accordingly, the cognitive intelligence platform 102 may modify the care plan 2350 to generate updated care plan 2320.1 based on the negative emotion. For example, the cognitive intelligence platform 102 may include at least one different health artifact in the updated care plan 2320.1 than was included in the care plan 2350. In some embodiments, a machine learning model may be trained to select health artifacts that historically improve a user's emotion when angry. Further, the cognitive intelligence platform 102 may remove the health artifacts determined to be associated with causing the negative emotion.

As depicted, the updated care plan 2320.1 includes new health artifacts represented by node “has complication” connected to nodes “Coronary Artery Disease”, “Diabetes Foot Problems”, “Diabetic Neuropathy”, and “Diabetic Retinopathy”. Further, the updated care plan 2320.1 removed the health artifacts represented by node “has symptom” connected to node “High Blood Sugar”. Providing the updated care plan 2320.1 may improve the experience of the user using the computing device 104 and may increase the likelihood the user continues to use the computing device 104.

FIG. 23C depicts an example updated care plan 2320.2. For purposes of explanation, the original care plan 2302 was the care plan 2350 depicted in FIG. 57C. The care plan 2350 may have been presented in the patient viewer on the user device 104. A physician may desire a certain medical outcome for the condition Type 2 Diabetes Mellitus. For example, the physician may desire to enhance the treatment of the medical condition. Accordingly, the physician may select various health artifacts to include in the updated care plan 2320.2. In the depicted example, the physician selected to include nodes represented as health artifacts “has self-care” connected to “Weight Management”, “Diabetic Diet”, “Healthy Eating”, “Diabetes Foot Care”, and “Being Active”. Information and/or action instructions may be generated and include in a natural language conversion of the updated care plan 2320.2 in the patient viewer, clinic viewer, and/or administrator viewer.

The updated care plan 2320.1 may be converted into natural language text by the cognitive intelligence platform 102 using the natural language database 122 according to the techniques disclosed herein. The cognitive intelligence platform 102 may generate action instructions pertaining to the health artifacts included in the care plan 2320.1. FIG. 23D depicts the care plan 2320.1 in the natural language text presented in a user interface 2360 of the patient viewer on the user device 104. Although the depicted natural language text is tailored for the patient, in some embodiments, the natural language text may be tailored for the medical personnel or the administrator when presented in the clinic viewer or the administrator viewer respectively.

It should be noted that the natural language text of the care plan 2320.1 depicted is an example and is for explanatory purposes. Any suitable variation of the natural language text is envisioned in this disclosure. The natural language text in the user interface 2360 presents “Please find information and/or action instructions relating to Type 2 Diabetes Mellitus below to Type 2 Diabetes Mellitus below:”.

For the “Medications” area and the “Tests” area, the natural language text is the same as described with reference to FIG. 23D.

As depicted, the “Symptoms” natural language text has been removed from the updated care plan 2320.1 and natural language text is added for health artifacts pertaining to the “Complications” and presented in the user interface 2360. The user interface 2360 presents information about types of complications for the condition: “Type 2 Diabetes Mellitus has complications of stroke, coronary artery disease, diabetes foot problems, diabetic neuropathy, diabetic retinopathy.” Further, the natural language text presents an action instruction for the patient: “Here is recommended medical content relating to those complications. Please read them.”. The action instruction may include links to the various recommended medical content. Further, the natural language text presents another action instruction: “Speak to your physician about the complications”.

The updated care plan 2320.2 may be converted into natural language text by the cognitive intelligence platform 102 using the natural language database 122 according to the techniques disclosed herein. The cognitive intelligence platform 102 may generate action instructions pertaining to the health artifacts included in the care plan 2320.2. FIG. 23E depicts the care plan 2320.2 in the natural language text presented in a user interface 2370 of the patient viewer on the user device 104. Although the depicted natural language text is tailored for the patient, in some embodiments, the natural language text may be tailored for the medical personnel or the administrator when presented in the clinic viewer or the administrator viewer respectively.

It should be noted that the natural language text of the care plan 2320.2 depicted is an example and is for explanatory purposes. Any suitable variation of the natural language text is envisioned in this disclosure. The natural language text in the user interface 2370 presents “Please find information and/or action instructions relating to Type 2 Diabetes Mellitus below to Type 2 Diabetes Mellitus below:”.

For the “Medications” area, the “Symptoms” area, and the “Tests” area, the natural language text is the same as described with reference to FIG. 23D.

As depicted, natural language text is added for health artifacts pertaining to the “Self-Care” and presented in the user interface 2370. As previously discussed, the health artifacts pertaining to “has self-care” were selected to be added based on the physician desiring a particular medical outcome. The user interface 2370 presents an action instruction for the patient: “Try self-care treatments for Type 2 Diabetes Mellitus including: weight management, diabetic diet, healthy eating, diabetes foot care, and being active.”

Clause 1. A method comprising:

receiving, at an artificial intelligence engine, a corpus of data for a patient, wherein the corpus of data includes a plurality of strings of characters;

identifying, in the plurality of strings of characters, indicia comprising a phrase, a predicate, a keyword, a subject, an object, a cardinal, a number, a concept, or some combination thereof;

comparing the indicia to a knowledge graph representing known health related information to generate a possible health related information pertaining to the patient;

identifying, using a logical structure, a structural similarity of the possible health related information and a known predicate in the logical structure; and

generating, by the artificial intelligence engine, cognified data based on the structural similarity.

Clause 2. The method of clause 2, further comprising generating the knowledge graph using the known health related information, wherein the knowledge graph represents knowledge of a disease and the knowledge graph comprises a plurality of concepts pertaining to the disease obtained from the known health related information, and the knowledge graph comprises relationships between the plurality of concepts.

Clause 3. The method of any preceding clause, wherein the cognified data comprises a health related summary of the possible health related information.

Clause 4. The method of any preceding clause, wherein generating, by the artificial intelligence engine, the cognified data further comprises:

generating at least one new string of characters representing a statement pertaining to the possible health related information; and

including the at least one new string of characters in the health related summary of the possible health related information.

Clause 5. The method of any preceding clause, wherein the statement describes an effect that results from the possible health related information.

Clause 6. The method of any preceding clause, further comprising codifying evidence based health related guidelines pertaining to a disease to generate the logical structure.

Clause 7. The method of any preceding clause, further comprising:

identifying at least one piece of information missing in the corpus of data for the patient using the cognified data, wherein the at least one piece of information pertains to a treatment gap, a risk gap, a quality of care gap, or some combination thereof; and

causing a notification to be presented on a computing device of a healthcare personnel, wherein the notification instructs entry of the at least one piece of information.

Clause 8. The method of any preceding clause, wherein using the logical structure to identify the structural similarity of the indicia and the known predicate in the logical structure further comprises identifying, based on the structural similarity of the indicia and the known predicate in the logical structure, a treatment pattern, a referral pattern, a quality of care pattern, a risk adjustment pattern, or some combination thereof in the corpus of data.

Clause 9. The method of any preceding clause, further comprising:

receiving feedback pertaining to whether the cognified data is accurate; and

updating the artificial intelligence engine based on the feedback.

Clause 10. The method of any preceding clause, a tangible, non-transitory computer-readable medium storing instructions that, when executed, cause a processing device to execute an artificial intelligence engine to:

receive a corpus of data for a patient, wherein the corpus of data includes a plurality of strings of characters;

identify, in the plurality of strings of characters, indicia comprising a phrase, a predicate, a keyword, a cardinal, a number, a concept, or some combination thereof;

compare the indicia to a knowledge graph representing known health related information to generate a possible health related information pertaining to the patient;

identify, using a logical structure, a structural similarity of the indicia and a known predicate in the logical structure; and

generate cognified data based on the similarity and the possible health related information.

Clause 11. The computer-readable medium of any preceding clause, wherein the artificial intelligence engine is further to generate the knowledge graph using the known health related information, wherein the knowledge graph represents knowledge of a disease and the knowledge graph comprises a plurality of concepts pertaining to the disease obtained from the known health related information, and the knowledge graph comprises relationships between the plurality of concepts.

Clause 12. The computer-readable medium of any preceding clause, wherein the cognified data comprises a health related summary of the possible health related information.

Clause 13. The computer-readable medium of any preceding clause, wherein generating, based on the pattern, the cognified data further comprises:

generating at least one new string of characters representing a statement pertaining to the possible health related information; and

including the at least one new string of characters in the health related summary of the possible health related information.

Clause 14. The computer-readable medium of any preceding clause, wherein the statement describes an effect that results from the possible health related information.

Clause 15. The computer-readable medium of any preceding clause, wherein the artificial intelligence engine is further to codify evidence based health related guidelines pertaining to a disease to generate the logical structure.

Clause 16. The computer-readable medium of any preceding clause, wherein the artificial intelligence engine is further to:

identify at least one piece of information missing in the corpus of data for the patient using the cognified data, wherein the at least one piece of information pertains to a treatment gap, a risk gap, a quality of care gap, or some combination thereof; and

cause a notification to be presented on a computing device of a healthcare personnel, wherein the notification instructs entry of the at least one piece of information.

Clause 17. The computer-readable medium of any preceding clause, wherein using the logical structure to identify the structural similarity of the indicia and the known predicate in the logical structure further comprises identifying, based on the structural similarity of the indicia and the known predicate in the logical structure, a treatment pattern, a referral pattern, a quality of care pattern, a risk adjustment pattern, or some combination thereof in the corpus of data.

Clause 18. The computer-readable medium of any preceding clause, wherein the artificial intelligence engine is further to:

receive feedback pertaining to whether the cognified data is accurate; and

update the artificial intelligence engine based on the feedback.

Clause 19. A system, comprising:

a memory device storing instructions; and

a processing device operatively coupled to the memory device, wherein the processing device executes the instructions to:

receive, at an artificial intelligence engine, a corpus of data for a patient, wherein the corpus of data includes a plurality of strings of characters;

identify, in the plurality of strings of characters, indicia comprising a phrase, a predicate, a keyword, a cardinal, a number, a concept, or some combination thereof;

compare the indicia to a knowledge graph representing known health related information to generate a possible health related information pertaining to the patient;

identify, using a logical structure, a structural similarity of the indicia and a known predicate in the logical structure; and

generate, by the artificial intelligence engine, cognified data based on the similarity and the possible health related information.

Clause 20. The system of any preceding claim, wherein the processing device is further to:

receive feedback pertaining to whether the cognified data is accurate; and

update the artificial intelligence engine based on the feedback.

Clause 21. The method of any preceding claim, wherein identifying the possible medical condition by identifying the similarity between the indicia and the knowledge graph further comprises using an artificial intelligence engine that is trained using feedback from medical personnel, wherein the feedback pertains to whether output regarding possible medical conditions from the artificial intelligence engine is accurate for input comprising notes of patients.

Clause 22. The method of any preceding claim, wherein the first information pertains to a name of the possible medical condition, a definition of the possible medical condition, or some combination thereof.

Clause 23. The method of any preceding claim, wherein identifying the possible medical condition by identifying the similarity between the indicia and the knowledge graph further comprises using a cognified data structure generated from the notes of the patient, wherein the cognified data structure includes a conclusion based on a logical structure representing codified evidence based guidelines pertaining to the possible medical condition.

Clause 24. The method of any preceding claim, wherein processing the patient notes to obtain the indicia further comprises inputting the notes into an artificial intelligence engine trained to identify the indicia in text based on commonly used indicia pertaining to the possible medical.

Clause 25. The computer-readable medium of any preceding clause, wherein detecting the possible medical condition by identifying the similarity between the indicia and the knowledge graph further comprises using an artificial intelligence engine that is trained using feedback from medical personnel, wherein the feedback pertains to whether output regarding possible medical, wherein detecting the possible medical condition by identifying the similarity between the indicia and the knowledge graph further comprises using a cognified data structure generated from the notes of the patient, and wherein the cognified data structure includes a conclusion about the predicate that is identified in a logic structure representing codified evidence based guidelines pertaining to the possible medical condition.

Clause 26. The computer-readable medium of any preceding clause, wherein processing the patient notes to obtain the indicia further comprises inputting the notes into an artificial intelligence engine trained to identify the indicia in text based on commonly used indicia pertaining to the possible medical condition.

Clause 27. A system, comprising:

a memory device storing instructions;

a processing device communicatively coupled to the memory device, the processing device executes the instructions to:
receive, at a server, an electronic medical record comprising notes pertaining to a patient;
process the notes to obtain indicia comprising a word, a cardinal, a phrase, a sentence, a predicate, or some combination thereof;
identify a possible medical condition of the patient by identifying a similarity between the indicia and a knowledge graph representing knowledge pertaining to the possible medical condition, wherein the knowledge graph comprises a plurality of nodes representing the plurality of information pertaining to the possible medical condition; and
provide, at a first time, first information of the plurality of information to a computing device of the patient for presentation on the computing device, the first information being associated with a root node of the plurality of nodes.

Clause 28. A method for diagnosing a medical condition through cognification of unstructured data, the method comprising:

receiving, at a server, an electronic medical record comprising notes pertaining to a patient;

generating cognified data using the notes, wherein the cognified data comprises a health summary of the medical condition;

generating, based on the cognified data, a diagnosis of the medical condition of the patient, wherein the diagnosis at least identifies a type of the medical condition; and

providing the diagnosis to a computing device for presentation on the computing device.

Clause 29. The method of any preceding clause, further comprising identifying, in the notes, indicia comprising a phrase, a predicate, a keyword, a cardinal, a number, a concept, or some combination thereof.

Clause 30. The method of any preceding clause, wherein generating the cognified data further comprises detecting the medical condition by identifying a similarity between the indicia and a knowledge graph.

Clause 31. The method of any preceding clause, further comprising using an artificial intelligence engine that is trained using feedback from medical personnel, wherein the feedback pertains to whether output regarding diagnoses from the artificial intelligence engine are accurate for input comprising notes of patients.

Clause 32. The method of any preceding clause, wherein the cognified data includes a conclusion that is identified based on a logic structure representing codified evidence based guidelines pertaining to the medical condition.

Clause 33. The method of any preceding clause, further comprising processing the notes to obtain indicia by inputting the notes into an artificial intelligence engine trained to identify the indicia in text based on commonly used indicia pertaining to the medical condition.

Clause 34. The method of any preceding clause, wherein generating the diagnosis further comprises:

determining a stage of the medical condition based on the cognified data; and

including the stage of the medical condition in the diagnosis.

Clause 35. The method of any preceding clause, further comprising:

determining a severity of the medical condition based on the stage and the type of the medical condition;

in response to the severity satisfying a threshold condition, providing a recommendation to seek immediate medical attention to a computing device of the patient.

Clause 36. A tangible, non-transitory computer-readable medium storing instructions that, when executed, cause a processing device to:

receive, at a server, an electronic medical record comprising notes pertaining to a patient;

generate cognified data using the notes, wherein the cognified data comprises a health summary of the medical condition;

generate, based on the cognified data, a diagnosis of the medical condition of the patient, wherein the diagnosis at least identifies a type of the medical condition; and

provide the diagnosis to a computing device for presentation on the computing device.

Clause 37. The computer-readable medium of any preceding clause, wherein the processing device is further to identify, in the notes, indicia comprising a phrase, a predicate, a keyword, a cardinal, a number, a concept, or some combination thereof.

Clause 38. The computer-readable medium of any preceding clause, wherein generating the cognified data further comprises detecting the medical condition by identifying a similarity between the indicia and a knowledge graph.

Clause 39. The computer-readable medium of any preceding clause, wherein the processing device is further to use an artificial intelligence engine that is trained using feedback from medical personnel, wherein the feedback pertains to whether output regarding diagnoses from the artificial intelligence engine are accurate for input comprising notes of patients.

Clause 40. The computer-readable medium of any preceding clause, wherein the cognified data includes a conclusion about a predicate in the notes that is identified in a logic structure representing codified evidence based guidelines pertaining to the medical condition.

Clause 41. The computer-readable medium of any preceding clause, wherein the processing device is further to process the patient notes to obtain indicia by inputting the notes into an artificial intelligence engine trained to identify the indicia in text based on commonly used indicia pertaining to the medical condition.

Clause 42. The computer-readable medium of any preceding clause, wherein generating the diagnosis further comprises:

determining a stage of the medical condition based on the cognified data; and

including the stage of the medical condition in the diagnosis.

Clause 43. The computer-readable medium of any preceding clause, wherein the processing device is further to:

determine a severity of the medical condition based on the stage and the type of the medical condition;

in response to the severity satisfying a threshold condition, provide a recommendation to seek immediate medical attention to a computing device of the patient.

Clause 44. A system, comprising:

a memory device storing instructions; and

a processing device communicatively coupled to the memory device, the processing device executes the instructions to:
receive, at a server, an electronic medical record comprising notes pertaining to a patient;
generate cognified data using the notes, wherein the cognified data comprises a health summary of the medical condition;
generate, based on the cognified data, a diagnosis of the medical condition of the patient, wherein the diagnosis at least identifies a type of the medical condition; and
provide the diagnosis to a computing device for presentation on the computing device.

Clause 45. The system of any preceding clause, wherein the processing device is further to identify, in the notes, indicia comprising a phrase, a predicate, a keyword, a cardinal, a number, a concept, or some combination thereof.

Clause 46. The system of any preceding clause, wherein generating the cognified data further comprises detecting the medical condition by identifying a similarity between the indicia and a knowledge graph.

Clause 47. The system of any preceding clause, wherein the processing device is further to use an artificial intelligence engine that is trained using feedback from medical personnel, wherein the feedback pertains to whether output regarding diagnoses from the artificial intelligence engine are accurate for input comprising notes of patients.

Clause 48. The computer-readable medium of any preceding clause, wherein the processing device is further to detect the detected tone of the patient based on words spoken by the patient, text entered by the patient, or some combination thereof.

Clause 49. The computer-readable medium of any preceding clause, wherein the processing device is further to detect the detected emotion of the patient based on words spoken by the patient, text entered by the patient, a detected facial expression of the patient, or some combination thereof.

Clause 50. A method, comprising:

receiving, by a device, patient data that indicates health related information associated with a patient;

identifying, by the device and by processing the patient data using one or more natural language processing techniques, indicia associated with a health status of the patient;

identifying, by the device, similarities between characteristics of the indicia and content characteristics of content that is part of a corpus of health related data;

generating, by the device and using an artificial intelligence engine, cognified data based on the similarities;

identifying, by the device, a medical code that correlates to particular content characteristics of the content that are similar to the characteristics of the indicia; and

causing, by the device, the cognified data to be displayed in association with the medical code.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

The word “example” is used herein to mean serving as an example, instance, or illustration. Any aspect or design described herein as “example” is not necessarily to be construed as preferred or advantageous over other aspects or designs. Rather, use of the word “example” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an implementation” or “one implementation” throughout is not intended to mean the same embodiment or implementation unless described as such.

Implementations the systems, algorithms, methods, instructions, etc., described herein can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors, or any other suitable circuit. In the claims, the term “processor” should be understood as encompassing any of the foregoing hardware, either singly or in combination. The terms “signal” and “data” are used interchangeably.

As used herein, the term module can include a packaged functional hardware unit designed for use with other components, a set of instructions executable by a controller (e.g., a processor executing software or firmware), processing circuitry configured to perform a particular function, and a self-contained hardware or software component that interfaces with a larger system. For example, a module can include an application specific integrated circuit (ASIC), a Field Programmable Gate Array (FPGA), a circuit, digital logic circuit, an analog circuit, a combination of discrete circuits, gates, and other types of hardware or combination thereof. In other embodiments, a module can include memory that stores instructions executable by a controller to implement a feature of the module.

Further, in one aspect, for example, systems described herein can be implemented using a general-purpose computer or general-purpose processor with a computer program that, when executed, carries out any of the respective methods, algorithms, and/or instructions described herein. In addition, or alternatively, for example, a special purpose computer/processor can be utilized which can contain other hardware for carrying out any of the methods, algorithms, or instructions described herein.

Further, all or a portion of implementations of the present disclosure can take the form of a computer program product accessible from, for example, a computer-usable or computer-readable medium. A computer-usable or computer-readable medium can be any device that can, for example, tangibly contain, store, communicate, or transport the program for use by or in connection with any processor. The medium can be, for example, an electronic, magnetic, optical, electromagnetic, or a semiconductor device. Other suitable mediums are also available.

The above-described embodiments, implementations, and aspects have been described in order to allow easy understanding of the present invention and do not limit the present invention. On the contrary, the invention is intended to cover various modifications and equivalent arrangements included within the scope of the appended claims, which scope is to be accorded the broadest interpretation to encompass all such modifications and equivalent structure as is permitted under the law.

What is claimed is:

1. A method, comprising:
receiving, by a device, patient data that indicates health related information associated with a patient;
identifying, by the device and by processing the patient data using one or more natural language processing techniques, indicia associated with a health status of the patient;
identifying, by the device, similarities between the indicia and content that is part of a corpus of health related data;
generating, by the device and using an artificial intelligence engine, cognified data based on the similarities;
identifying, by the device, a medical code that correlates to particular content that is similar to the indicia; and
causing, by the device, the cognified data to be displayed in association with the medical code.

2. The method of claim 1, wherein identifying the similarities comprises:
comparing the indicia with the content, where the content is stored using a knowledge graph, and
identifying a semantic or semantically-related similarity between a characteristic of the indicia and a corresponding content characteristic; and
wherein generating the cognified data comprises:
generating the cognified data based on the semantic or semantically-related similarity.

3. The method of claim 1, wherein identifying the similarities comprises:
comparing the indicia with the content, where the content is stored using a knowledge graph, and
identifying, using a logical structure, a structural similarity of the indicia and a known predicate of the logical structure of the knowledge graph; and
wherein generating the cognified data comprises:
generating the cognified data based on the structural similarity.

4. The method of claim 1, wherein generating the cognified data comprises:
identifying, using the artificial intelligence engine, a pattern based on a structural similarity between a logical structure of a data structure used to store the indicia and a logical structure of a knowledge graph used to store the content, and
generating the cognified data based on the pattern.

5. The method of claim 1, wherein generating the cognified data comprises:
generating the cognified data in real-time or near real-time relative to receiving the health related information;
wherein identifying the medical code comprises:
identifying the medical code in real-time or near real-time relative to receiving the health related information; and
wherein causing the cognified data to be displayed comprises:
causing the cognified data to be displayed in association with the medical code in real-time or near real-time relative to receiving the health related information.

6. The method of claim 1, wherein the cognified data provides a summary of the health status for the patient and includes at least one of:
a conclusion,
a recommendation,
a complication,
a risk statement,
a description of a cause of a health complication, or
a description of symptoms of the health complication.

7. The method of claim 1, further comprising:
determining, using the cognified data, that particular indicia represents new health information that is not found in the corpus of health related data; and
causing a data structure to be updated with the new health information.

8. A device, comprising:
one or more processors; and
one or more memories including instructions that, when executed by the one or more processors, cause the one or more processors to:
receive patient data that indicates health related information associated with a patient;
identify, by processing the patient data using one or more natural language processing techniques, indicia associated with a health status of the patient;
identify similarities between the indicia and the content that is part of a corpus of health related data;
generate, using an artificial intelligence engine, cognified data based on the similarities;
identify a medical code that correlates to the content having the content characteristics similar to the characteristics of the indicia; and
cause the cognified data to be displayed in association with medical codes that relate to at least the indicia or the cognified data.

9. The device of claim 8, wherein the one or more processors, when identifying the similarities, are to:
compare the indicia with the content, where the content is stored using a knowledge graph, and
identify a semantic or semantically-related similarity between a characteristic of the indicia and a corresponding content characteristic; and
wherein the one or more processors, when generating the cognified data, are to:
generate the cognified data based on the semantic or semantically-related similarity.

10. The device of claim 8, wherein the one or more processors, when identifying the similarities, are to:
compare the indicia with the content, where the content is stored using a knowledge graph, and
identify, using a logical structure, a structural similarity of the indicia and a known predicate of the logical structure of the knowledge graph; and
wherein the one or more processors, when generating the cognified data, are to:
generate the cognified data based on the structural similarity.

11. The device of claim 8, wherein the one or more processors, when generating the cognified data, are to:
identify, using the artificial intelligence engine, a pattern based on a structural similarity between a logical structure of a data structure used to store the indicia and a logical structure of a knowledge graph used to store the content, and
generate the cognified data based on the pattern.

12. The device of claim 8, wherein the one or more processors, when generating the cognified data, are to:
generate the cognified data in real-time or near real-time relative to receiving the health related information.

13. The method of claim 1, wherein the cognified data provides a summary of the health status for the patient and includes at least one of:
a conclusion,
a recommendation,
a complication,
a risk statement,
a description of a cause of a health complication, or
a description of symptoms of the health complication.

14. The device of claim 8, wherein the one or more processors are further to:
determine, using the cognified data, that particular indicia represents new health information that is not found in the corpus of health related data; and
cause a data structure to be updated with the new health information.

15. A non-transitory computer-readable medium storing instructions, the instructions comprising:
one or more instructions that, when executed by one or more processors, cause the one or more processors to:
receive patient data that indicates health related information associated with a patient;
identify, by processing the patient data using one or more natural language processing techniques, indicia associated with a health status of the patient;
identify similarities between the indicia and content that is part of a corpus of health related data;
generate, using an artificial intelligence engine, cognified data based on the similarities;
identify a medical code that correlates to the content having the content characteristics similar to the characteristics of the indicia; and
cause the cognified data to be displayed in association with the medical code.

16. The non-transitory computer-readable medium of claim 15, wherein the one or more instructions, that cause the one or more processors to identify the similarities, cause the one or more processors to:
compare the indicia with the content, where the content is stored using a knowledge graph, and
identify a semantic or semantically-related similarity between a characteristic of the indicia and a corresponding content characteristic; and
wherein the one or more instructions, that cause the one or more processors to generate the cognified data, cause the one or more processors to:
generate the cognified data based on the semantic or semantically-related similarity.

17. The non-transitory computer-readable medium of claim 15, wherein the one or more instructions, that cause the one or more processors to identify the similarities, cause the one or more processors to:
compare the indicia with the content, where the content is stored using a knowledge graph, and
identify, using a logical structure, a structural similarity of the indicia and a known predicate of the logical structure of the knowledge graph; and
wherein the one or more instructions, that cause the one or more processors to generate the cognified data, cause the one or more processors to:
generate the cognified data based on the structural similarity.

18. The non-transitory computer-readable medium of claim 15, wherein the one or more instructions, that cause the one or more processors to identify the similarities, cause the one or more processors to:
identify, using the artificial intelligence engine, a pattern based on a structural similarity between a logical structure of a data structure used to store the indicia and a logical structure of a knowledge graph used to store the content, and
generate the cognified data based on the pattern.

19. The non-transitory computer-readable medium of claim 15, wherein the one or more instructions, that cause the one or more processors to identify the similarities, cause the one or more processors to:
generate the cognified data in real-time or near real-time relative to receiving the health related information.

20. The non-transitory computer-readable medium of claim 15, wherein the one or more instructions, when executed by the one or more processors, further cause the one or more processors to:
determine, using the cognified data, that particular indicia represents new health information that is not found in the corpus of health related data; and
cause a data structure to be updated with the new health information.
